(19) United States
(12) Patent Application Publication
(10) Pub. No.: US 2025/0049862 A1
(43) Pub. Date: Feb. 13, 2025

(54) SPRAY DRIED INHALABLE BIOTHERAPEUTICS FOR THE TREATMENT OF DISEASE

(71) Applicant: Alveolus Bio, Inc., Birmingham, AL (US)

(72) Inventor: Andrew O'Connor, Birmingham, AL (US)

(21) Appl. No.: 18/717,237

(22) PCT Filed: Dec. 15, 2022
(86) PCT No.: PCT/US2022/052936

§ 371 Date: Jun. 6, 2024

Related U.S. Application Data
(60) Provisional application No. 63/290,289, filed on Dec. 16, 2021.

(51) Int. Cl. A61K 35/744; A61K 9/00; A61K 9/16; A61K 9/48; A61K 45/06; A61P 11/00; A61P 31/16

(52) U.S. Cl. CPC A61K 35/744; A61K 9/0075; A61K 9/1623; A61K 9/1682; A61K 9/4858; A61K 45/06; A61P 11/00; A61P 31/16

(57) ABSTRACT

The technology described herein is directed to spray-dried biotherapeutic matrix compositions comprising a bacterial preparation, which is formulated for administration by inhalation. Also described herein unit dosage forms of such spray-dried biotherapeutic matrix compositions, devices comprising such pharmaceutical compositions, methods of producing such pharmaceutical compositions, and methods of treating diseases, such as bronchopulmonary diseases, among others, using such spray-dried biotherapeutic matrix compositions.

### CROSS-REFERENCE TO RELATED APPLICATIONS

This application claims benefit under 35 U.S.C. § 119(e) of U.S. Provisional Application No. 63/290,289 filed Dec. 16, 2021, the contents of which are incorporated herein by reference in their entirety.

### TECHNICAL FIELD

The technology described herein relates to spray-dried inhalable biotherapeutics for the treatment of disease.

### BACKGROUND

Inhaled drug delivery poses advantages over other routes of administration in that therapeutics delivered directly to the lung have been shown to have improved clinical efficacy when compared to other routes of administration; see e.g., Jackson (1995) British Journal of General Practice, 45 (401): 683:687; the contents of which are incorporated herein by reference in its entirety. Because of this improved clinical efficacy, many drugs have been formulated to be inhaled by patients to achieve reduced drug load requirements for efficacy, resulting in lower systemic exposure and reduced side-effects.

There are many routes of administering drugs that can be used to treat indications such as a chronic bronchopulmonary disorder—including parenteral administration (e.g., XOLAIR—omalizumab), intravenous delivery (e.g., CINQAIR—reslizumab), oral capsule (e.g., DALIRESP—roflumilast), metered dose inhaler (e.g., Ventolin—albuterol), liquid nebulizer (e.g., XOPENEX—levalbuterol), and dry powder inhalers (e.g., SPIRIVA—tiotropium). Some drugs (such as ROFLUMILAST for COPD) administered systemically via ingestion pose a higher risk for side effects due to systemic exposure and gastro-intestinal interactions; see e.g., Garnock-Jones, Drugs 75, 1645-1656 (2015); the contents of which are incorporated herein by reference in its entirety. Drugs prescribed for use via injection are typically approved for specific phenotypes of asthma or psoriasis, which can be difficult to diagnose, and do not include other chronic respiratory diseases.

Nebulized therapeutics are typically given to patients in a small container of drug solution. That container is then inserted into a nebulizing device which creates a mist of drug solution for the patient to inhale, usually over a sustained period of inhalation. This route of administration is useful for dosing patients who are unable to powerfully inhale (children, infants, elderly). However, drugs formulated in a nebulizing solution have stability challenges that can make new drug development challenging.

To avoid some of the stability issues with formulation drugs for use in a nebulizer system, pressurized inhalers have been developed for use in inhaling therapeutics. These metered dose inhalers are commonly used for the delivery of bronchodilators and corticosteroids in patients suffering from asthma and COPD. Originally, these pressurized inhalers worked by pressurizing a container with drug solution and chlorofluorocarbon. Over time, it was discovered that inhaling CFCs affects the central nervous system, so more recent MDIs have utilized hydrofluoroalkane to pressurize canisters and deliver therapeutics; see e.g., Muralidharan et al. (2015) Expert Opinion on Drug Delivery, 12:6, 947-962; the contents of which are incorporated herein by reference in its entirety.

Dry powder inhalers have been used more recently in the past decade to deliver drugs that otherwise would be difficult to formulate in nebulizers or MDIs—drugs such as tiotropium bromide and itraconazole. These therapeutics take the form of a dry, aerosolize-able powder in a breakable container (such as a cellulose-based capsule or sachet). The patient breaks the drug product container and inhales the dry powder through the broken enclosure via a mouthpiece attached to the dry powder inhaler itself. An exemplary dry powder inhaler device is the PLASTIAPE RS01 inhaler—it is generally available and a good candidate for delivery development research. In this inhaler, the drug product capsule sits in a chamber at the base of the inhaler. The user presses two “triggers” on either side of the inhaler to break the capsule. The user then turns the inhaler horizontally and inhales through the mouthpiece, causing the powder contents of the capsule to empty while the capsule rotates quickly, increasing the amount of powder dosed to the user. Many inhaled dry powder treatments utilize custom inhalers with pre-loaded powders into chambers corresponding to the dosing regimen (such as SPRIVIA HANDIHALER, or the BREO ELLIPTA).

This route of delivery poses numerous advantages over other routes of administration, including more predictable particle fraction delivered to the lungs, improved drug stability, the ability to use hydrophobic active ingredients, and the absence of chemical propellants. The use of dry powder inhalers includes therapies to treat chronic obstructive pulmonary disease (COPD), asthma, and pulmonary infections. Along with these pulmonary diseases, additional indications can benefit from dry powder delivery of therapeutics, such as idiopathic pulmonary fibrosis, cystic fibrosis, bronchiectasis, chronic cough, or infectious respiratory diseases. Furthermore, disease profiles beyond chronic respiratory diseases can benefit from dosing to the lungs including central nervous system (CNS) diseases such as Parkinson's disease and migraines, osteoporosis, and pulmonary hypertension; see e.g., Noymer et al. (2011) Ther Deliv 2 (9):1125-1140; Abdou et al. (2019) Drug Deliv 26 (1):689-699; Yu et al. (2021) J Control Release 338:486-504; Hill et al. (2015) Respir Care 60 (6):794-802; discussion 802-795; Hosang et al. Nature 603: 138-144 (2022); the contents of each of which are incorporated herein by reference in their entireties.

Dysbiosis in the lungs can trigger chronic neutrophilic inflammation. Upon an insult to the airway epithelium, collagen is exposed and cleaved by Matrix Metalloproteinase 9 (MMP-9). MMP-9 and prolyl endopeptidase (PE) cleave the collagen fragments to liberate the prolyl-glycyl-proline (PGP) peptide PGP in its acetylated form (Ac-PGP). Ac-PGP triggers neutrophilic inflammation by binding receptors on CXC chemokine receptor 2 (CXCR2). Both live bacteria and their extracts and metabolites can modulate the pathway leading to Ac-PGP production. Many chronic and infectious bronchopulmonary disorders are marked by symptoms from the body's inflammatory response. Dysbiosis has shown to be a contributing factor in chronic inflammatory pulmonary diseases including asthma, cystic fibrosis, Bronchopulmonary Dysplasia, and Chronic Obstructive Pulmonary Disease. See e.g., Gaggar et al. (2008) J Immunol 180 (8):5662-5669; Malik et al. (2007) J Immunol 178 (2):1013-1020; Lin et al. (2008) Am J Pathol 173 (1):144-153; Weathington et al. (2006) Nature Medicine 12 (3):317-323.; Hilty et al. (2010) PLoS One 5 (1):e8578; Marri et al. (2013) J Allergy Clin Immunol 131 (2):346-352 e341-343; Zhao et al. (2012) Proc Natl Acad Sci USA 109 (15):5809-5814; Coburn et al. (2015) Sci Rep 5:10241; Lal et al. (2016) Scientific Reports 6 (1):31023; Bowerman et al. (2020) Nat Commun 11 (1):5886; Huang et al. (2014) J Clin Microbiol 52 (8):2813-2823; the contents of each of which are incorporated herein by reference in their entireties.

As such, there is a need to develop easily administrable therapeutics for chronic inflammatory pulmonary diseases.

### SUMMARY

The present disclosure describes the unexpected finding that biotherapeutics, including microbiota and/or extracts or metabolites thereof, can be formulated for administration by inhalation. Accordingly, the technology described herein is directed to spray-dried biotherapeutic matrix compositions comprising a bacterial preparation, which is formulated for administration by inhalation. Also described herein are unit dosage forms of such spray-dried biotherapeutic matrix compositions, devices comprising such pharmaceutical compositions, methods of producing such pharmaceutical compositions, and methods of treating diseases, such as bronchopulmonary diseases, among others, using such spray-dried biotherapeutic matrix compositions.

In one aspect, described herein is a spray-dried biotherapeutic matrix composition comprising a bacterial preparation, wherein the matrix composition is formulated for administration by inhalation.

In some embodiments of any of the aspects, the bacterial preparation comprises viable or non-viable bacteria.

In some embodiments of any of the aspects, the viable bacteria are capable of actively metabolizing and/or proliferating in the lung of a subject.

In some embodiments of any of the aspects, the non-viable bacteria are heat-killed.

In some embodiments of any of the aspects, bacteria are Gram negative.

In some embodiments of any of the aspects, bacteria are Gram positive.

In some embodiments of any of the aspects, the bacteria are spore-forming.

In some embodiments of any of the aspects, the bacteria are in spore form.

In some embodiments of any of the aspects, the bacteria are aerobic.

In some embodiments of any of the aspects, the bacteria are anaerobic.

In some embodiments of any of the aspects, the bacteria produce at least one immunomodulator.

In some embodiments of any of the aspects, the bacteria belong to a genus selected from the group consisting of: Carnobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella.

In some embodiments of any of the aspects, the bacterium is Lacticaseibacillus rhamnosus, Lactobacillus acidophilus, or Lactiplantibacillus plantarum.

In some embodiments of any of the aspects, the bacteria are non-pathogenic.

In some embodiments of any of the aspects, the bacteria are present at a concentration of at least 10¹ colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g.

In some embodiments of any of the aspects, the bacteria are present at a concentration of at least 10⁶ colony-forming units per gram (CFU/g).

In some embodiments of any of the aspects, the bacteria are present at a concentration of at least 10⁸ colony-forming units per gram (CFU/g).

In some embodiments of any of the aspects, the bacteria are resistant to at least one antibiotic.

In some embodiments of any of the aspects, the composition comprises at least 0.5% bacterial preparation by dry weight.

In some embodiments of any of the aspects, the bacterial preparation comprises a bacterial extract or bacterial metabolite preparation.

In some embodiments of any of the aspects, the bacterial extract or bacterial metabolite preparation is selected from the group consisting of: a bacterial exosome; bacterial cell wall; peptidoglycan; teichoic acid; lipoteichoic acid; bacterial S-layer; exopolysaccharide; polysaccharide; a lactic acid polymer; a lactic acid derivative; a lactic acid intermediate; hydrogen peroxide; a bacteriocin; a salivaricin; a reuterin; and a bacterial growth supernatant.

In some embodiments of any of the aspects, the composition further comprises at least one excipient.

In some embodiments of any of the aspects, the composition further comprises at least two excipients.

In some embodiments of any of the aspects, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

In some embodiments of any of the aspects, the excipient is leucine and/or trehalose.

In some embodiments of any of the aspects, the composition comprises at least 0.5% excipient by dry weight.

In some embodiments of any of the aspects, the composition further comprises at least one stabilizer.

In some embodiments of any of the aspects, the stabilizer comprises a surfactant.

In some embodiments of any of the aspects, the stabilizer is a polysorbate; poloxamer; or polyvinyl alcohol.

In some embodiments of any of the aspects, the stabilizer is Polysorbate 20, Polysorbate 40, Polysorbate 60, or Polysorbate 80.

In some embodiments of any of the aspects, the stabilizer is Polysorbate 80.

In some embodiments of any of the aspects, the stabilizer is Poloxamer 184, Poloxamer 185, Poloxamer 188, Poloxamer 234, Poloxamer 235, Poloxamer 238, Poloxamer 333, Poloxamer 334, Poloxamer 335, Poloxamer 338, Poloxamer 403, or Poloxamer 407.

In some embodiments of any of the aspects, the composition comprises at least 0.25% of the stabilizer or stabilizers by dry weight.

In some embodiments of any of the aspects, the composition comprises at least one excipient and at least one stabilizer.

In some embodiments of any of the aspects, the composition further comprises at least one additional therapeutic.

In some embodiments of any of the aspects, the at least one additional therapeutic is selected from the group consisting of: an anti-inflammatory, an antimicrobial, an antiviral, an antifungal, a vasodilator, and a bronchodilator.

In some embodiments of any of the aspects, the at least one additional therapeutic is incorporated into the composition using microencapsulation, co-formulation, or covalent linkage to the composition with a degradable linker.

In some embodiments of any of the aspects, the matrix composition comprises a plurality of dried particles.

In some embodiments of any of the aspects, the dried particles have a Dv50 of at least 0.5 μm.

In some embodiments of any of the aspects, the dried particles have a median mass aerodynamic diameter (MMAD) of at least 1.5 μm to at most 7.5 μm.

In some embodiments of any of the aspects, the dried particles have a dispersibility of less than 2.0.

In some embodiments of any of the aspects, the dried particles have a dispersibility of at least 0.5 to 1.0.

In some embodiments of any of the aspects, the dried particles have a delivered dose of at least 25.0% to at most 125% of the bacterial preparation by mass to a target tissue.

In some embodiments of any of the aspects, the dried particles have a delivered dose of at least 60% of the bacterial preparation by mass to a target tissue.

In some embodiments of any of the aspects, the target tissue is a target bronchopulmonary tissue.

In some embodiments of any of the aspects, the target bronchopulmonary tissue is the lungs, the trachea, the bronchi, the bronchioles, and/or the alveoli.

In some embodiments of any of the aspects, the target tissue is a distal tissue site from the lungs delivered via the cardiovascular system or lymphatic system.

In some embodiments of any of the aspects, the dried particles have a bulk density of at least 0.1 g/cm³ to 0.8 g/cm³.

In some embodiments of any of the aspects, the dried particles have a bulk density of at least 0.5 g/cm³.

In some embodiments of any of the aspects, the dried particles have a tapped density of at least 0.1 g/cm³ to 1.0 g/cm³.

In some embodiments of any of the aspects, the dried particles have a tapped density of at least 0.6 g/cm³.

In some embodiments of any of the aspects, the dried particles have a moisture content of at least 1.0% to 7.0% water by weight by Karl Fischer.

In some embodiments of any of the aspects, the dried particles have a moisture content of at least 2.5% water by weight by Karl Fischer.

In some embodiments of any of the aspects, the composition is formulated for delivery to the trachea, the bronchi, the bronchioles, and/or the alveoli.

In some embodiments of any of the aspects, the composition is formulated for delivery to the lungs.

In some embodiments of any of the aspects, the composition is formulated as a capsule.

In some embodiments of any of the aspects, the capsule contains at least 10 mg of the spray-dried biotherapeutic matrix composition.

In some embodiments of any of the aspects, the composition is formulated for delivery by an inhaler.

In some embodiments of any of the aspects, the composition is formulated for delivery by a dry powder inhaler (DPI), a metered dose inhaler (MDI), or a soft-mist inhaler (SMI).

In some embodiments of any of the aspects, the composition is in combination with an inhaler.

In one aspect, described herein is an inhalation device for bronchopulmonary delivery comprising: (a) an inhaler; and (b) a container containing a spray-dried biotherapeutic matrix composition comprising a bacterial preparation.

In some embodiments of any of the aspects, the inhaler is a dry powder inhaler (DPI), a metered dose inhaler (MDI), or a soft mist inhaler (SMI).

In some embodiments of any of the aspects, the inhaler comprises: (a) a mouthpiece comprising an opening; and (b) means for aerosolizing or dispersing the spray-dried biotherapeutic matrix composition in the container.

In one aspect, described herein is a method of preparing a spray-dried biotherapeutic matrix composition comprising a bacterial preparation, comprising: (a) preparing a liquid feedstock comprising the bacterial preparation; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the spray-dried biotherapeutic matrix composition.

In one aspect, described herein is a method of preparing a spray-dried biotherapeutic matrix composition comprising a bacterial preparation, comprising: (a) obtaining a liquid feedstock comprising the bacterial preparation; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the spray-dried biotherapeutic matrix composition.

In some embodiments of any of the aspects, the step of preparing the liquid feedstock comprises dissolving a solid feedstock into an aqueous solution.

In some embodiments of any of the aspects, the solid feedstock comprises: (a) at least 3.5% bacterial preparation by weight; (b) at least 5% excipient by weight; and/or (c) at least 0.25% stabilizer by weight.

In some embodiments of any of the aspects, the solid feedstock comprises: (a) at least 3.5% bacterial preparation by weight; (b) at least 5% of a first excipient by weight; (c) at least 5% of a second excipient by weight; and/or (d) at least 0.25% stabilizer by weight.

In some embodiments of any of the aspects, the solid feedstock comprises at least 1% to at most 5% bacterial preparation by weight.

In some embodiments of any of the aspects, the solid feedstock comprises at least 45% to at most 95% excipient by weight.

In some embodiments of any of the aspects, the solid feedstock comprises at least 5%-60% of a first excipient by weight, and at least 5%-60% of a second excipient by weight.

In some embodiments of any of the aspects, the solid feedstock comprises at least 0.25% to at most 10% stabilizer by weight.

In some embodiments of any of the aspects, the liquid feedstock comprises at least 1 g/L solid feedstock dissolved in an aqueous solution.

In some embodiments of any of the aspects, the liquid feedstock comprises at least 25 g/L solid feedstock dissolved in an aqueous solution.

In some embodiments of any of the aspects, the liquid feedstock comprises at least 0.1% to at most 10% solid feedstock dissolved in an aqueous solution.

In some embodiments of any of the aspects, the liquid feedstock comprises at least 1% solid feedstock dissolved in an aqueous solution.

In some embodiments of any of the aspects, 1 L of the liquid feedstock comprises: (a) at least 1.050 g bacterial preparation; (b) at least 1.5 g excipient; (b) at least 0.075 g stabilizer; and/or (c) at least 970 g aqueous solution.

In some embodiments of any of the aspects, 1 L of the liquid feedstock comprises: (a) at least 1.050 g bacterial preparation; (b) at least 0.75 g of a first excipient; (c) at least 0.75 g of a second excipient; (d) at least 0.075 g stabilizer; and/or (e) at least 970 g aqueous solution.

In some embodiments of any of the aspects, 1 L of the liquid feedstock comprises: (a) at least 1.050 g bacterial preparation; (b) at least 0.5 g of a first excipient; (c) at least 0.5 g of a second excipient; (d) at least 0.5 g of a third excipient; (e) at least 0.075 g stabilizer; and/or (f) at least 970 g aqueous solution.

In some embodiments of any of the aspects, 1 L of the liquid feedstock comprises at least 750 g to at most 999 g aqueous solution.

In some embodiments of any of the aspects, the liquid feedstock comprises: (a) at least 0.105% bacterial preparation; (b) at least 0.15% excipient; (c) at least 0.0075% stabilizer; and/or (d) at least 97% aqueous solution.

In some embodiments of any of the aspects, the liquid feedstock comprises: (a) at least 0.105% bacterial preparation by weight; (b) at least 0.075% of a first excipient by weight; (c) at least 0.075% of a second excipient by weight; (d) at least 0.0075% stabilizer and/or (e) at least 97% aqueous solution by weight.

In some embodiments of any of the aspects, the liquid feedstock comprises: (a) at least 0.105% bacterial preparation by weight; (b) at least 0.05% of a first excipient by weight; (c) at least 0.05% of a second excipient by weight; (d) at least 0.05% of a third excipient by weight; (e) at least 0.0075% stabilizer and/or (f) at least 97% aqueous solution by weight.

In some embodiments of any of the aspects, the liquid feedstock comprises at least 0.01% to at most 10% bacterial preparation by weight.

In some embodiments of any of the aspects, the liquid feedstock comprises at least 1.0% oto at most 20% excipient by weight.

In some embodiments of any of the aspects, the liquid feedstock comprises at least 0.1% to at most 19.8% of a first excipient by weight, and at least 0.1% to at most 19.8% of a second excipient by weight.

In some embodiments of any of the aspects, the liquid feedstock comprises at least 0.1% to at most 19.8% of a first excipient by weight, at least 0.1% to at most 19.8% of a second excipient by weight, and at least 0.1% to at most 19.8% of a third excipient by weight.

In some embodiments of any of the aspects, the liquid feedstock comprises at least 0.01% to at most 1.0% stabilizer by weight.

In some embodiments of any of the aspects, the liquid feedstock comprises at least 75% to at most 99.9% aqueous solution by weight.

In some embodiments of any of the aspects, the bacterial preparation comprises viable or non-viable bacteria.

In some embodiments of any of the aspects, the bacteria belong to a genus selected from the group consisting of: Carnobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella.

In some embodiments of any of the aspects, the bacterium is Lacticaseibacillus rhamnosus, Lactobacillus acidophilus, or Lactiplantibacillus plantarum.

In some embodiments of any of the aspects, the bacteria are non-pathogenic.

In some embodiments of any of the aspects, the bacteria are present at a concentration of at least 10¹ colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g.

In some embodiments of any of the aspects, the bacteria are present at a concentration of at least 10⁶ colony-forming units per gram (CFU/g).

In some embodiments of any of the aspects, the bacteria are present at a concentration of at least 10⁸ colony-forming units per gram (CFU/g).

In some embodiments of any of the aspects, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

In some embodiments of any of the aspects, the excipient is leucine and/or trehalose.

In some embodiments of any of the aspects, the stabilizer is a polysorbate; poloxamer; or polyvinyl alcohol.

In some embodiments of any of the aspects, the stabilizer is Polysorbate 80.

In some embodiments of any of the aspects, the aqueous solution is water.

In some embodiments of any of the aspects, the liquid feedstock further comprises at least one additional therapeutic.

In some embodiments of any of the aspects, the at least one additional therapeutic is selected from the group consisting of: an anti-inflammatory, an antimicrobial, an antiviral, an antifungal, a vasodilator, and a bronchodilator.

In some embodiments of any of the aspects, the atomization nozzle into the drying chamber has a diameter of at least 1.2 um.

In some embodiments of any of the aspects, the droplets of liquid feedstock produced by the atomization nozzle into the drying chamber have a diameter of at least 1.5 um.

In some embodiments of any of the aspects, the droplets of liquid feedstock have a flow rate through the drying chamber of at least 0.5 g/min.

In some embodiments of any of the aspects, the droplets of liquid feedstock have a flow rate through the drying chamber of at least 15 g/min.

In some embodiments of any of the aspects, the droplets of liquid feedstock have a flow rate through the drying chamber of at most 1000 g/min.

In some embodiments of any of the aspects, the heated, pressurized gas is heated before being inlet into the drying chamber.

In some embodiments of any of the aspects, the heated, pressurized gas is inlet into the drying chamber at a temperature of at least 100° C.

In some embodiments of any of the aspects, the heated, pressurized gas is inlet into the drying chamber at a temperature of at most 195° C.

In some embodiments of any of the aspects, the heated, pressurized gas is outlet from the drying chamber at a temperature of at least 40° C.

In some embodiments of any of the aspects, the heated, pressurized gas is outlet from the drying chamber at a temperature of at least 48° C.

In some embodiments of any of the aspects, the heated, pressurized gas is outlet from the drying chamber at a temperature of at most 85° C.

In some embodiments of any of the aspects, the heated, pressurized gas is pressurized before being inlet into the drying chamber.

In some embodiments of any of the aspects, the heated, pressurized gas in the drying chamber has an atomization gas pressure of at least 10 pounds per square inch gauge (psig).

In some embodiments of any of the aspects, the heated, pressurized gas in the drying chamber has an atomization gas pressure of at least 20 pounds per square inch gauge (psig).

In some embodiments of any of the aspects, the heated, pressurized gas in the drying chamber has an atomization gas pressure of at most 150 pounds per square inch gauge (psig).

In some embodiments of any of the aspects, the heated, pressurized gas has a flow rate through the drying chamber of at least 5 kg/hr.

In some embodiments of any of the aspects, the heated, pressurized gas has a flow rate through the drying chamber of at least 18 kg/hr.

In some embodiments of any of the aspects, the heated, pressurized gas has a flow rate through the drying chamber of at most 150 kg/hr.

In some embodiments of any of the aspects, the heated, pressurized gas is outlet through the cyclone chamber.

In some embodiments of any of the aspects, the step of exposing the liquid feedstock droplets to heated, pressurized gas in the drying chamber takes at most 8 hours.

In some embodiments of any of the aspects, the dried particles isolated in the cyclone chamber have a median mass aerodynamic diameter (MMAD) of at least 1.5 μm to at most 7.5 μm.

In some embodiments of any of the aspects, the dried particles isolated in the cyclone chamber have a median mass aerodynamic diameter (MMAD) of at least 4.0 μm.

In some embodiments of any of the aspects, the step of isolating dried particles of a predetermined range of diameters in the cyclone chamber occurs continuously.

In some embodiments of any of the aspects, the spray-dried biotherapeutic matrix composition comprises a bacterial viability of at least 5% after the step of isolating the dried particles.

In one aspect, described herein is a method of delivering a spray-dried biotherapeutic matrix composition comprising a bacterial preparation to a subject, comprising: (a) obtaining an inhalation device for bronchopulmonary delivery comprising: (i) an inhaler; and (ii) a container containing a spray-dried biotherapeutic matrix composition comprising a bacterial preparation; (b) activating the inhaler to cause aerosolization or dispersal of the spray-dried biotherapeutic matrix composition; and (c) inhaling the aerosolized or dispersed spray-dried biotherapeutic matrix composition.

In one aspect, described herein is a method of delivering a spray-dried biotherapeutic matrix composition comprising a bacterial preparation to a subject, comprising: (a) obtaining an inhalation device for bronchopulmonary delivery comprising: (i) an inhaler; and (ii) a container containing a spray-dried biotherapeutic matrix composition as described herein; (b) activating the inhaler to cause aerosolization or dispersal of the spray-dried biotherapeutic matrix composition; and (c) inhaling the aerosolized or dispersed spray-dried biotherapeutic matrix composition.

In one aspect, described herein is a method of delivering a spray-dried biotherapeutic matrix composition comprising a bacterial preparation to a subject, comprising: (a) obtaining an inhalation device as described herein; (b) activating the inhaler to cause aerosolization or dispersal of the spray-dried biotherapeutic matrix composition; and (c) inhaling the aerosolized or dispersed spray-dried biotherapeutic matrix composition.

In some embodiments of any of the aspects, the inhaler is a dry powder inhaler (DPI), a metered dose inhaler (MDI), or a soft mist inhaler (SMI).

In some embodiments of any of the aspects, the inhaler comprises: (a) a mouthpiece comprising an opening; and (b) means for aerosolizing or dispersing the spray-dried biotherapeutic matrix composition in the container.

In some embodiments of any of the aspects, the inhaler has an inspiration flow rate of at least 15 L/min.

In some embodiments of any of the aspects, at least 25% to at most 100.0% of the spray-dried biotherapeutic matrix composition by mass is delivered to a target bronchopulmonary tissue.

In some embodiments of any of the aspects, at least 60.0% of the spray-dried biotherapeutic matrix composition by mass is delivered to a target bronchopulmonary tissue.

In some embodiments of any of the aspects, the target bronchopulmonary tissue is the lungs, the trachea, the bronchi, the bronchioles, and/or the alveoli.

In some embodiments of any of the aspects, the spray-dried biotherapeutic matrix composition is delivered from the bronchopulmonary tissue to a distal tissue site via the cardiovascular system or lymphatic system.

In one aspect, described herein is a method of treating a subject in need thereof comprising administering through inhalation an effective dose of a spray-dried biotherapeutic matrix composition comprising a bacterial preparation.

In one aspect, described herein is a method of treating a subject in need thereof comprising administering through inhalation an effective dose of a spray-dried biotherapeutic matrix composition as described herein.

In some embodiments of any of the aspects, the subject has been diagnosed with or is at risk of developing a chronic bronchopulmonary disease.

In some embodiments of any of the aspects, the chronic bronchopulmonary disease is selected from the group consisting of: chronic obstructive pulmonary disease (COPD), lung cancer, asthma, bronchiectasis, emphysema, cystic fibrosis (CF), bronchopulmonary dysplasia (BPD), acute respiratory disease syndrome (ARDS), idiopathic pulmonary fibrosis (IPF), pulmonary hypertension (PAH), silicosis, interstitial lung disease (ILD), and pleural effusion (PE).

In some embodiments of any of the aspects, the lung cancer is small cell lung cancer (SCLC) or non-small cell lung cancer (NSCLC).

In some embodiments of any of the aspects, the subject has been diagnosed with or is at risk of contracting an infectious pulmonary disease.

In some embodiments of any of the aspects, the infectious bronchopulmonary disease is caused by or associated with an infectious agent selected from: adenovirus; coronavirus; influenza virus; parainfluenza virus; parvovirus; respiratory syncytial virus; rhinovirus; enterovirus; measles virus; rubella virus; varicella virus; Corynebacterium diphtheriae; Haemophilus influenzae; Legionella pneumophila; Bordetella pertussis; Mycobacterium tuberculosis; Streptococcus species; Pseudomonas species; Escherichia coli; Aspergillus species; Cryptococcus species; and Pneumocystis species.

In some embodiments of any of the aspects, the spray-dried biotherapeutic matrix composition is administered in conjunction with a standard of care for the chronic or infectious bronchopulmonary disease.

In some embodiments of any of the aspects, the spray-dried biotherapeutic matrix composition is administered using an inhaler.

In some embodiments of any of the aspects, the inhaler is a dry powder inhaler (DPI), a metered dose inhaler (MDI), or a soft mist inhaler (SMI).

In some embodiments of any of the aspects, the effective dose of the spray-dried biotherapeutic matrix composition is at least 10⁴ CFU of bacteria per unit dose.

In some embodiments of any of the aspects, the effective dose of the spray-dried biotherapeutic matrix composition is at least 10⁴ CFU of viable bacteria per unit dose.

In some embodiments of any of the aspects, the spray-dried biotherapeutic matrix composition reduces neutrophilic inflammation in a target tissue.

In some embodiments of any of the aspects, the spray-dried biotherapeutic matrix composition increases lactic acid concentration in a target tissue by at least 25%.

In some embodiments of any of the aspects, the method further comprises administering at least one additional therapeutic.

In some embodiments of any of the aspects, the at least one additional therapeutic is selected from the group consisting of: an anti-inflammatory, an antimicrobial, an antiviral, an antifungal, a vasodilator, and a bronchodilator.

In some embodiments of any of the aspects, the spray-dried biotherapeutic matrix composition comprises the bacterial preparation and the at least one additional therapeutic.

In some embodiments of any of the aspects, the spray-dried biotherapeutic matrix composition is co-administered with the at least one additional therapeutic.

In some embodiments of any of the aspects, the co-administration comprises administering using a combination delivery device.

In one aspect, described herein is a unit dosage form comprising at least 1 mg of a spray-dried biotherapeutic matrix composition comprising a bacterial preparation.

In one aspect, described herein is a unit dosage form comprising at least 1 mg of a spray-dried biotherapeutic matrix composition as described herein.

In one aspect, described herein is a unit dosage form comprising at least 1 mg of the spray-dried biotherapeutic matrix composition prepared by a method as described herein.

In one aspect, described herein is a unit dosage form comprising at least 1 mg of a spray-dried biotherapeutic matrix composition comprising at least 10⁴ CFU bacteria per unit dose.

In some embodiments of any of the aspects, the dosage is at least 30 mg spray-dried biotherapeutic matrix composition.

In some embodiments of any of the aspects, the dosage comprises at least 10⁴ CFU of bacteria per unit dose.

In some embodiments of any of the aspects, the dosage comprises at least 10⁴ CFU of viable bacteria per unit dose.

### BRIEF DESCRIPTION OF THE DRAWINGS

FIG. 1 is a schematic of an exemplary spray drying machine and associated method, as described further herein.

FIG. 2 is an image of an exemplary dry powder inhaler.

FIG. 3 is a bar graph showing the assaying of three bacterial strains AB101, AB102, and AB103 for their growth and viability in vitro over the course of 24 hours. The left-right order of the bars corresponds to the top-down order of the graph legend.

FIG. 4A-4C is a series of bar graphs and a table showing the testing of healthy mice inoculated intratracheally with a blend of live bacterial strains AB101, 102, and 103 in a 1:1:1 ratio or a negative control. FIG. 4A is a bar graph showing the bacterial load in the lung tissue of the mice at 0-hours, 4-hours, 8-hours, 12-hours, 16-hours, 24-hours, and 72-hours post-inoculation. FIG. 4B is a table showing the bacterial load from FIG. 4A. FIG. 4C is a bar graph showing the lactic acid output in the bronchoalveolar lavage (BAL) fluid of the mice at the indicated time points.

FIG. 5A-5B is a series of tables showing testing of spray dried powder comprising bacteria for bacterial viability. F4 solution contained 0.05% Polysorbate 80 in water for injection, and F6 media contained 5:55 MRS:F4. Liquid feedstock solution was the solution pre-drying. FIG. 5A is table showing the spray-drying conditions for each batch. FIG. 5B is table showing the viability of the bacteria in the spray-dried powder for each batch. As used herein, “API” stands for active pharmaceutical ingredient.

FIG. 6 is a table of particle characteristics of two exemplary formulations of spray dried live biotherapeutic product AB1000 (AB1000 comprises a spray-dried formulation of the AB101, AB102, and AB103 strains, including excipients; see e.g., Table 12).

FIG. 7 is a table showing the viability of two spray dried formulations of AB1000 in the spray-dried powder for each batch as measured by International Organization for Standardization (ISO) 7889 enumeration standard. ISO 7889 specifies a method for the enumeration of characteristic microorganisms in yogurt by means of the colony-count technique at 37 degrees Celsius.

FIG. 8 is a table showing the resistance and susceptibility profiles of live bacterial strains AB101, AB102, and AB103 to 51 antibiotics.

FIG. 9 is a table showing the group allocation for testing mice exposed to porcine pancreatic elastase (PPE) with or without lipopolysaccharide (LPS) dosed with representative drug powder of live biotherapeutic AB1000. This testing scheme is referred to herein as “PPE model mice.”

FIG. 10A-10B is a series of images and bar graphs showing changes in lung structure of mice groups in the PPE model. FIG. 10A is a series of histology images showing changes in lung structure from the mice groups delineated in FIG. 9. FIG. 10B is a series of bar graphs showing radial alveolar count (RAC) and mean linear intercept (MLI) of mouse lung tissue from FIG. 10A.

FIG. 11 is a bar graph of pulmonary function testing using lung resistance measured in cm H₂O/mL/s in the PPE model mice.

FIG. 12 is a bar graph showing the mRNA transcription levels of MMP-9 in lung tissue of the PPE model mice.

FIG. 13A-13E is a series of bar graphs showing changes in protein levels of markers of inflammation in the bronchoalveolar lavage (BAL) fluid of the PPE model mice. FIG. 13A is a bar graph showing the significant decrease of MMP-9 protein in both PPE and PPE+LPS mice treated with AB1000. FIG. 13B is a bar graph showing the significant decrease of neutrophil elastase (NE) protein in the BAL of PPE+LPS mice treated with AB1000. FIG. 13C is a bar graph showing the significant decrease of C-reactive protein (CRP) in PPE+LPS mice treated with AB1000. FIG. 13D is a bar graph showing the significant decrease of interleukin 8 (IL-8) in PPE and PPE+LPS mice treated with AB1000. FIG. 13E is a bar graph showing the significant increase of immunoglobulin A (IgA) in PPE and PPE+LPS mice treated with AB1000.

FIG. 14A-14E is a series of bar graphs showing changes in protein levels of markers of inflammation in the serum of the PPE model mice. FIG. 14A is a bar graph showing the significant decrease of MMP-9 protein in PPE mice treated with AB1000. FIG. 14B is a bar graph showing the significant decrease of MMP-9 protein in PPE+LPS mice treated with AB1000. FIG. 14C is a bar graph showing the significant decrease of NE protein in PPE mice treated with AB1000. FIG. 14D is a bar graph showing the significant decrease of NE protein PPE+LPS mice treated with AB1000. FIG. 14E is a bar graph showing the significant decrease of C-reactive protein (CRP) in PPE+LPS mice treated with AB1000.

FIG. 15A-15B is a series of images and bar graphs showing the change in lung tissue structure in mice exposed to PPE and LPS and those treated with AB1000 or fluticasone furoate, an inhaled steroid. FIG. 15A is a series of histology images showing changes in lung tissue structure across exposure and treatment groups. FIG. 15B is a bar graph of mean linear intercept (MLI) showing improvement in tissue structure upon treatment with AB1000.

FIG. 16 is a bar graph showing that AB1000 and fluticasone furoate performed comparably in reducing MMP-9 expression in lung tissue of mice exposed to PPE and LPS.

FIG. 17A-17I is a series of bar graphs showing significant increases in protein levels of anti-inflammatory cytokines Exodus 2 (FIG. 17A), Macrophage Inflammatory protein-3b (MIP-3b) (FIG. 17B), interleukin-11 (IL-11) (FIG. 17C), monocyte chemotactic protein 5 (MCP-5) (FIG. 17D), thymus- and activation-regulated chemokine (TARC) (FIG. 17E), MIP-3a (FIG. 17F), IL-16 (FIG. 17G), tissue inhibitor of metalloproteinases 1 (TIMP1) (FIG. 17H), and macrophage-derived chemokine (MDC) (FIG. 17I) in the bronchoalveolar lavage fluid (BAL) of mice exposed to PPE+LPS which were treated with AB1000.

FIG. 18 is a series of dot plots showing a significant decrease in MMP-9 expression in lung tissue, a significant decrease in MMP-9 protein in serum, and a significant increase in IgA protein in the BAL of mice exposed to cigarette smoke or control air treated with AB1000 or control saline treatment.

FIG. 19A-19B is a series of bar graphs showing that bacterial administration (e.g., AB101, AB102, and/or AB103) reduced expression of markers of fibrogenesis and markers of fibrosis development. FIG. 19A is a series of bar graphs showing significant decreases in expression of markers of fibrogenesis (smooth muscle alpha-actin (αSMA)) and fibrosis development (collagen type I alpha 1 (COL1A1), COL1A2, fibronectin) in vitro in LL29 idiopathic pulmonary fibrosis (IPF) fibroblasts treated with Lacto blend; “Lacto blend” refers to the blend of Lactobacillus bacteria strains AB101, AB102, and AB103; also referred to herein interchangeably as “AB blend”; see e.g., Table 12). FIG. 19B is a series of bar graphs showing that the blend of strains (AB101, AB102, AB103) performed better than individual strains in reducing fibrotic markers.

FIG. 20A-20B is a series of images and a bar graph showing that Lacto blend slowed the epithelial to mesenchymal transition in bleomycin-exposed human bronchial epithelial cells. FIG. 20A is a series of images measuring the wound healing rate 17 hours after scratch of the cells. FIG. 20B is a bar graph showing the measured wound healing rates from FIG. 20A.

FIG. 21 is a series of bar graphs showing that Lacto blend reduced profibrotic markers (transforming growth factor-beta-1 (TGFbeta-1), found in inflammatory zone protein 1 (FIZZ1; also referred to as Resistin-like molecule alpha) and upregulated anti-fibrotic markers (IL-6, tumour necrosis factor alpha (TNF-alpha)) in THP1 monocytes (a human leukemia monocytic cell line) exposed to bleomycin.

FIG. 22 is a series of bar graphs showing that Lacto blend reduced influenza A (IAV) virus hemagglutinin (HA) protein and C-reactive protein (CRP) in BAL of IAV-infected mice.

FIG. 23 is a series of bar graphs showing that Lacto blend (“Lacto”) reduced markers of neutrophilic inflammation in a double hit hyperoxia (HO)+E. coli murine model of bronchopulmonary dysplasia (BPD). Measured markers of neutrophilic inflammation included: MMP-9 mRNA in lung tissue and the following proteins in BAL: MMP-9, myeloperoxidase (MPO), NE, CRP, and IL-6.

FIG. 24 is a table showing the resistance of a blend of live bacterial strains AB101, AB102, and AB103 to antibiotics (see also FIG. 8).

### DETAILED DESCRIPTION

The present disclosure describes the unexpected finding that biotherapeutics, including microbiota and/or extracts or metabolites thereof, can be formulated for administration by inhalation. Accordingly, the technology described herein is directed to spray-dried biotherapeutic matrix compositions comprising a bacterial preparation, which is formulated for administration by inhalation. Also described herein are unit dosage forms of such spray-dried biotherapeutic matrix compositions, devices comprising such pharmaceutical compositions, methods of producing such pharmaceutical compositions, and methods of treating diseases, such as bronchopulmonary disease, among others, using such spray-dried biotherapeutic matrix compositions.

Providing a dose of commensal bacteria to the lungs of a chronic disease sufferer can have significant benefits to the patient, whether this means direct colonization of the patient's lungs with bacteria (e.g., live biotherapeutic), or dosing of bacterial extracts and/or metabolites (e.g., biotherapeutic). Dysbiosis occurs when there is an imbalance of commensal (beneficial) and pathogenic (harmful) bacteria. The basis for dosing commensal bacteria to address dysbiosis for positive health benefits has been well-established through the practice of fecal matter transplants (FMT). Data also indicate that active bacterial extracts and metabolites can produce anti-inflammatory effects systemically. See e.g., Jang et al. (2020) Experimental & Molecular Medicine 52 (7):1128-1139; Weingarden and Vaughn B P (2017) Gut Microbes 8 (3):238-252; Arpaia et al. (2013) Nature 504 (7480):451-455; Iraporda et al. (2015) Immunobiology 220 (10):1161-1169; Vinolo et al. (2011) Nutrients 3 (10):858-876; the contents of each of which are incorporated herein by reference in their entireties.

### Preparation and Delivery Formats for Therapeutics Comprising Bacterial Preparations

Delivery of a biotherapeutic directly to the lungs requires an inhaled dosing technique, such as nebulized delivery, a metered dose inhaler (MDI), a dry powder inhaler (DPI), or a soft mist inhaler (SMI). Dosing live biotherapeutics, in particular, is challenging due to stability concerns. The presence of moisture makes storage and use of a powdered live biotherapeutic a challenge. Typically, during manufacture of a live organism (usually by fermentation), the “finishing” step of production includes freeze drying, or lyophilization, to remove water from the powdered organisms while in a cold state to maximize longevity. One can consider the use of a nebulizer for delivery of bacteria or bacterial products via inhalation, but where a nebulizer uses a liquid (i.e., water-containing) form of the drug preparation, stability remains an issue, as does the fact that typical nebulizers require the use of a small mesh to create droplets for aerosolization. Many live biotherapeutics, in particular bacteria, have a size too large to reliably pass through this mesh, creating additional challenges for the use of a nebulizer to deliver bacteria or bacterial products.

In a metered dose inhaler, many interactions are at play—namely, the drug contents to be dosed being stored under elevated pressure with propellants. Stability in this case is also a concern, not only for live organisms but sensitive biotherapeutics that have been carefully extracted and contain specific genes and proteins requiring gentle processing. While propellants and high pressures are not generally conducive to maintaining viability of bacteria, MDIs can be used to deliver for non-viable bacteria (e.g., heat-killed bacteria), bacterial extracts, and/or bacterial products.

To avoid these challenges, delivery of an inhalable dry powder through a DPI can be used. However, a therapeutic must be formulated as an inhalable dry powder to be used in a DPI successfully. This powder must possess specific moisture contents (usually very low, between 1 and 5%) for stability purposes, as well as particular aerodynamic properties to ensure the powder can be delivered properly and reliably. As detailed further herein, the process known as spray drying can be used to “engineer” this type of dry powder for use in a DPI.

### Spray Drying

Spray drying is a technique through which multiple raw materials are dissolved, atomized into droplets, and dried quickly in a drying chamber to create a custom, dry, aerosolize-able powder with tuned particle characteristics. Spray drying can be used for production of inhaled dry powders to treat diseases such as COPD, cystic fibrosis (CF), asthma, as well as non-respiratory disorders such as diabetes and migraines.

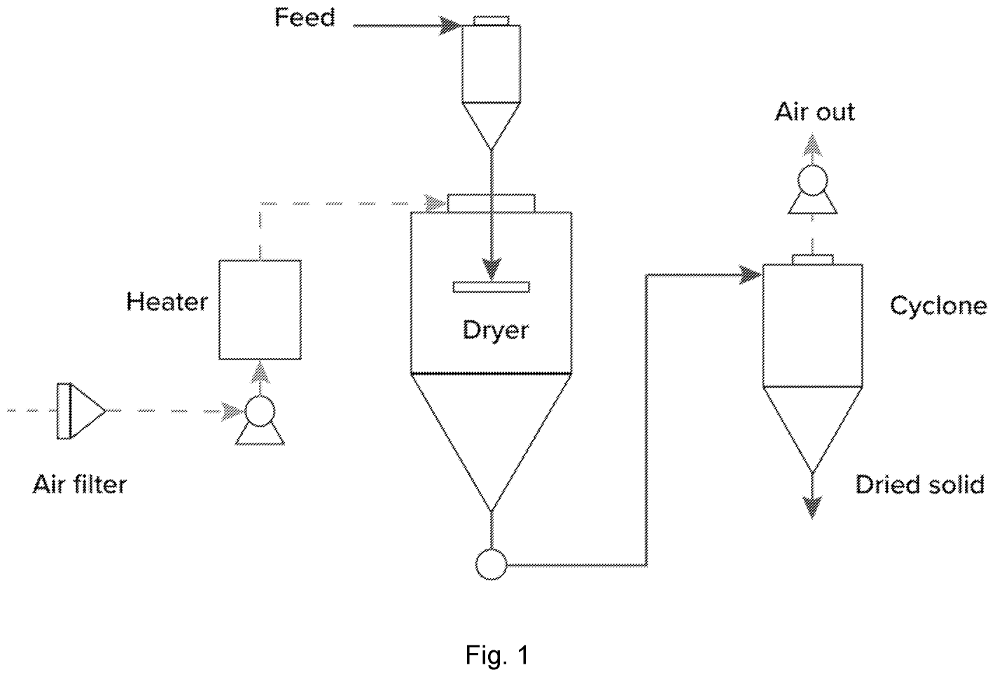

Spray drying involves the creation of a feedstock containing dissolved solid ingredients intended to be present in the dry particle at some defined concentration (e.g., a percent ratio of solute weight to solvent weight, % w/w). The feedstock is then fed through a nozzle at a specified pressure to create a droplet. That droplet is dried by heated gas running through the drying chamber to quickly create a dry particle. That particle is then collected at the bottom of a cyclone—a device designed to capture a reduced range of particle diameters, letting the rest of the “waste” particles be collected at the end of the process. These cyclone-captured particles represent a drug product bulk powder, intended or designed to be inhaled for the treatment or prevention of disease. See e.g., FIG. 1 for a schematic of an exemplary spray drying process.

The production of an effective spray-dried powder can include more components than simply the active pharmaceutical ingredient itself. The powder can include the active ingredient, one or more excipients, residual solvent, and/or an emulsification stabilizer. These ingredients are dissolved or suspended in the feedstock to be dried prior to the start of spray drying. The homogenous solution or suspension is then dried to create the inhalable dry powder itself.

The included excipients are used to provide a number of benefits to the spray dried powder. Namely, they are included to provide specific thermodynamic and physical properties. The excipients are often responsible for the shape of the spray dried particle itself due to their solubility properties. In the feedstock solvent, the solubility of the excipient determines how quickly the sprayed droplet forms a solid particle, and how quickly the solid molecules move toward the center of the droplet during drying. Once the particle is dried, the chemical and thermodynamic characteristics of the powder and their stability affect the solubility of the final powder, namely through the polymorphism (or lack thereof) of the final powder over time and across temperature and humidity exposures. Good excipient selection results in a dry powder exhibiting stable crystallinity, high particle density, consistent shape, and high dispersibility. Frequently, amino acids such as leucine are combined with a salt or sugar to optimize for this effect.

Surfactant stabilizers are frequently used when spray drying formulations with hydrophobic or insoluble particles. In some embodiments, the formulation includes a stabilizer when spray drying a suspension so that the suspension emulsifies and disperses evenly, allowing for a homogeneous suspension from which to spray consistent droplets. These surfactants are often included at low rates to minimally affect the final dry powder. Polysorbates of varying purity are frequently used, but other organic acid combinations are possible, along with steric surfactants, such as PLURONIC F68.

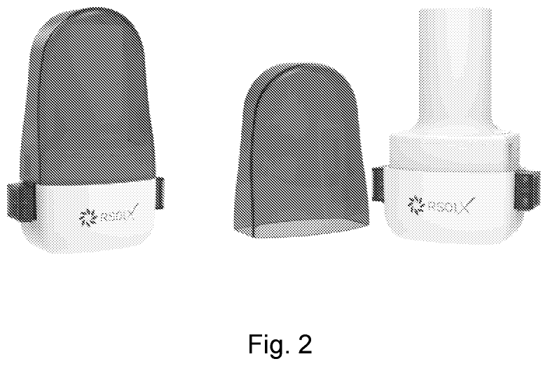

These spray dried powders can be encapsulated and used in some form of dry powder delivery device through which a patient inhales the particles deeply, allowing for settling into target depths in the airways. These spray dried powders can be filled into break-able capsules or sachets and broken open at the time of use inside of the delivery device. Once the enclosure is broken, the patient inhales deeply through the mouthpiece on their inhaler, allowing the powder to deposit into the patient's throat, esophagus, and lungs. The intention of a dry powder for inhalation is to create a powder of a certain aerodynamic size and density such that a predictable, safe dose of drug deposits in the appropriate portion of the lung airway. See e.g., FIG. 2 for a non-limiting example of an inhaled dry powder delivery device.

Described herein is a drug delivery mechanism by which a patient can dose their lungs directly with a biotherapeutic (e.g., alive, non-living, extract, or polymerized metabolite) via inhalation of a spray dried powder for the treatment of a chronic bronchopulmonary disorder, e.g., marked by inflammation. The powder contains all or some of the constituents in a drug delivery matrix, as exemplified in Table 1A-1C or Table 10A-10B, below.

TABLE 1A
Exemplary spray-dried biotherapeutic matrix composition,
30 mg capsule fill (e.g., dry powder for DPI)

| Constituents of Spray | % by | |
|---|---|---|
| Dried Drug Product | weight | mg |
| Bacterial Preparation | 25.0% | 7.500 |
| Excipient 1 | 34.2% | 10.26 |
| Excipient 2 | 34.2% | 10.26 |
| Excipient 3 | 5.00% | 1.500 |
| Stabilizer (e.g., an | 1.60% | 0.480 |
| emulsifying stabilizer) | | |

TABLE 1B
Exemplary spray-dried biotherapeutic matrix composition,
30 mg capsule fill (e.g., dry powder for DPI)

| Constituents of Spray | % by | |
|---|---|---|
| Dried Drug Product | weight | mg |
| Bacterial Preparation | 25.0% | 7.20 |
| Excipient 1 | 34.17% | 9.840 |
| Excipient 2 | 34.17% | 9.840 |
| Excipient 3 | 5.00% | 1.440 |
| Stabilizer (e.g., an | 1.67% | 0.480 |
| emulsifying stabilizer) | | |

TABLE 1C
Exemplary bacterial preparation (e.g., representing the
25.0% bacterial preparation shown in Tables 1A-1B).

| Constituents of Bacteria | % by |
|---|---|
| API Preparation | weight |
| Lactobacillus plantarum | 1.26 |
| Lactobacillus acidophilus | 5.07 |
| Lactobacillus rhamnosus | 2.07 |
| Filler/binder | 9.14 |
| (e.g., GLUCIDEX) | |
| Trehalose | 5.48 |
| Ascorbic acid | 0.87 |
| Oligosaccharide | 0.47 |
| (e.g., betacyclodextrin) | |
| Water | 0.62 |

Table 10A: Minimum constituent concentrations in an exemplary spray-dried biotherapeutic matrix composition, (each constituent represents an exemplary minimum % value that can be made up for the other constituents in the composition; e.g., a low % of bacterial preparation can be counteracted by higher than minimum percentage of excipient 1, excipient 2, and/or stabilizer.

| Constituents of Spray | % by |
|---|---|
| Dried Drug Product | weight |
| Bacterial Preparation | 0.5% |
| Excipient 1 | 0.25% |
| Excipient 2 | 0.25% |
| Stabilizer (e.g., an | 0.5% |
| emulsifying stabilizer) | |

Table 10B: Minimum constituent concentrations in an exemplary spray-dried biotherapeutic matrix composition, (each constituent represents an exemplary minimum % value that can be made up for the other constituents in the composition; e.g., a low % of bacterial preparation can be counteracted by higher than minimum percentage of excipient 1, excipient 2, excipient 3, and/or stabilizer.

| Constituents of Spray Dried Drug Product | % by weight |
|---|---|
| Bacterial Preparation | 0.5% |
| Excipient 1 | 0.25% |
| Excipient 2 | 0.25% |
| Excipient 3 | 0.25% |
| Stabilizer (e.g., an emulsifying stabilizer) | 0.5% |

### Spray-Dried Biotherapeutic Matrix Compositions

Described herein is an inhaled biotherapeutic drug product that delivers a matrix of constituents directly to the lungs for the treatment of chronic diseases. In one aspect, described herein is a spray-dried biotherapeutic matrix composition comprising a bacterial preparation, wherein the matrix composition is formulated for administration by inhalation. As used herein, the phrase “spray-dried biotherapeutic matrix composition” refers to a composition comprising a matrix of biotherapeutic that can be produced using a spray-drying process as described herein. The present disclosure encompasses embodiments of this inhaled biotherapeutic drug product—either in dry crystalline solid form, dry amorphous solid form, or a mixture—containing a matrix of one or more of the following constituents: (a) a bacterial preparation; (b) at least one excipient; and/or (c) at least one stabilizer.

In some embodiments, the spray-dried biotherapeutic matrix composition comprises: (a) a bacterial preparation. In some embodiments, the spray-dried biotherapeutic matrix composition comprises: (b) at least one excipient. In some embodiments, the spray-dried biotherapeutic matrix composition comprises: (c) at least one stabilizer. In some embodiments, the spray-dried biotherapeutic matrix composition comprises: (a) a bacterial preparation; and (b) at least one excipient. In some embodiments, the spray-dried biotherapeutic matrix composition comprises: (a) a bacterial preparation; and (c) at least one stabilizer. In some embodiments, the spray-dried biotherapeutic matrix composition comprises: (b) at least one excipient; and (c) at least one stabilizer. In some embodiments, the spray-dried biotherapeutic matrix composition comprises: (a) a bacterial preparation; (b) at least one excipient; and (c) at least one stabilizer.

### Bacterial Preparation

The spray-dried biotherapeutic matrix composition as described herein comprises at least one bacterial preparation. In some embodiments, the spray-dried biotherapeutic matrix composition comprises at least 1, 2, 3, 4, 5, 6, 7, 8, 9, 10, or more bacterial preparations. As used herein, the term “bacterial preparation” refers to a preparation comprising living bacteria (i.e., “live biotherapeutic”) and/or non-living bacteria or components thereof (i.e., “non-living biotherapeutic”).

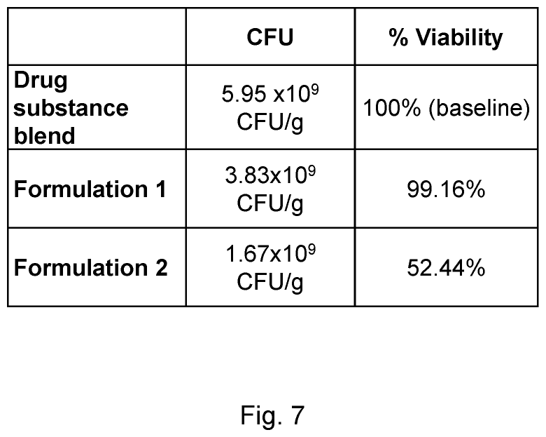

In some embodiments, the bacterial preparation comprises viable or non-viable bacteria. In some embodiments, the bacterial preparation comprises viable bacteria. In some embodiments, the viable bacteria are capable of actively metabolizing and/or proliferating in the lung of a subject. In some embodiments, the bacterial preparation (e.g., in the spray-dried biotherapeutic matrix composition; e.g., after spray-drying) comprises bacteria with a viability of at least 50%, at least 60%, at least 70%, at least 80%, at least 90%, at least 95%, or at least 99% (see e.g., FIG. 7). In some embodiments, the bacterial preparation comprises non-viable bacteria. In some embodiments, the non-viable bacteria are heat-killed. In some embodiments, the non-viable bacteria are killed with ultraviolet light, ethanol, or bleach. In some embodiments, the non-viable bacteria are killed with antibiotics.

In some embodiments, the bacteria are aerobic. In some embodiments, the bacteria are anaerobic. In some embodiments, the bacteria are aerobes. In some embodiments, the bacteria are obligate aerobes. In some embodiments, the bacteria are anaerobes. In some embodiments, the bacteria are obligate anaerobes. In some embodiments, the bacteria are facultative anaerobes. In some embodiments, the bacteria are aerotolerant anaerobes. In some embodiments, the bacteria are capnophiles. In some embodiments, the bacteria are microaerophiles.

In some embodiments, the bacteria produce at least one immunomodulator. As used herein, the term “immunomodulator” refers a substance that affects the functioning of the immune system, e.g., increases or decreases immune function. It should be understood that a bacterium or its components can raise or provoke an adaptive immune response that generates antibodies against the bacterium or its components; while this is technically affecting the functioning of the immune system, the immunomodulation encompassed by an “immunomodulator” as used herein does not encompass an adaptive response that raises antibodies. Rather, an “immunomodulator” produced by a bacterium as described herein will generally modulate an innate immune function including but not limited to inflammatory responses (or suppression thereof), cytokine or chemokine production (or suppression thereof), and the like.

In some embodiments, the bacterial immunomodulator increases immune function. Non-limiting examples of bacterial immunomodulators that increase immune function include bacterial toxins (e.g., cholera toxin, pertussis toxin, etc.), flagellin, or lipopolysaccharide (LPS). Such a bacterial immunomodulator that increases immune function can be used in indications involving immunocompromisation, infection, or cancer, or any other need for an increased immune response. In some embodiments, the bacterial immunomodulator decreases immune function. Non-limiting examples of bacterial immunomodulators that decrease immune function include lipopeptides (e.g., amphomycins, polymyxins, teicoplanins, or bacitracin) or daptomycin, which can suppress inflammatory cytokine expression. Such a bacterial immunomodulator that decreases immune function can be used in indications involving autoimmunity, cytokine storm, or any other need for a decreased immune response.

In some embodiments, the bacteria are Gram negative. In some embodiments, the bacteria are Gram positive. In some embodiments, the bacteria are acid-fast. In some embodiments, the bacteria are spore-forming. In some embodiments, the bacteria are in spore form. In some embodiments, the spray-dried biotherapeutic matrix composition comprises at least one (e.g., at least 1, at least 2, at least 3, at least 4, at least 5, at least 6, at least 7, at least 8, at least 9, at least 10, or more) of the bacterial preparations listed in Table 11. In some embodiments, the spray-dried biotherapeutic matrix composition comprises three bacterial preparations.

TABLE 11
Exemplary Bacterial Preparations

| Live Biotherapeutic (genus) | Non-living Biotherapeutic |
|---|---|
| Carnobacterium | Bacterial exosomes |
| Lactiplantibacillus | Peptidoglycan |
| Lactobacillus | Teichoic acid |
| Lacticaseibacillus | Lipoteichoic acid |
| Ligilactobacillus | Bacterial S-layer |
| Oenococcus | Exopolysaccharide |
| Leuconostoc | Polysaccharides |
| Pedicoccus | Lactic Acid Polymers/derivatives/ |
| | intermediates |
| Enterococcus | Hydrogen Peroxide |
| Lactococcus | Bacteriocin |
| Staphylococcus | Salivaricin |
| Streptococcus | Reuterin |
| Streptomyces | Bacterial growth supernatant |
| Bifidobacterium | Heat-killed bacteria |
| Propionibacterium | Non-viable bacteria |
| Moraxella | Bacterial cell wall |

In some embodiments, the bacteria belong to a genus selected from the group consisting of: Camobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella. In some embodiments, the bacteria belong to the genus Camobacterium. In some embodiments, the bacteria belong to the genus Lactiplantibacillus. In some embodiments, the bacteria belong to the genus Lactobacillus. In some embodiments, the bacteria belong to the genus Lacticaseibacillus. In some embodiments, the bacteria belong to the genus Ligilactobacillus. In some embodiments, the bacteria belong to the genus Oenococcus. In some embodiments, the bacteria belong to the genus Leuconostoc. In some embodiments, the bacteria belong to the genus Pedicoccus. In some embodiments, the bacteria belong to the genus Enterococcus. In some embodiments, the bacteria belong to the genus Lactococcus. In some embodiments, the bacteria belong to the genus Staphylococcus. In some embodiments, the bacteria belong to the genus Streptococcus. In some embodiments, the bacteria belong to the genus Streptomyces. In some embodiments, the bacteria belong to the genus Bifidobacterium. In some embodiments, the bacteria belong to the genus Propionibacterium. In some embodiments, the bacteria belong to the genus Moraxella.

In some embodiments, the bacteria are Lacticaseibacillus rhamnosus, Lactobacillus acidophilus, or Lactiplantibacillus plantarum. In some embodiments, the bacteria are Lacticaseibacillus rhamnosus (previously referred to as Lactobacillus rhamnosus). In some embodiments, the bacteria are Lactobacillus acidophilus. In some embodiments, the bacteria are Lactiplantibacillus plantarum (previously referred to as Lactobacillus plantarum). In some embodiments, the bacteria are Lacticaseibacillus rhamnosus and Lactobacillus acidophilus. In some embodiments, the bacteria are Lacticaseibacillus rhamnosus and Lactiplantibacillus plantarum. In some embodiments, the bacteria are Lactobacillus acidophilus and Lactiplantibacillus plantarum. In some embodiments, the bacteria are Lacticaseibacillus rhamnosus, Lactobacillus acidophilus, and Lactiplantibacillus plantarum. In some embodiments, the bacteria are Lacticaseibacillus rhamnosus strain LGG. In some embodiments, the bacteria are Lactiplantibacillus plantarum ATCC BAA-793™ In some embodiments, the bacteria are Lactobacillus acidophilus ATCC 4356™. In some embodiments, the bacteria are Lacticaseibacillus rhamnosus ATCC 53103™. In some embodiments, the bacteria are lactic acid bacteria (LAB), i.e., belong to the order Lactobacillales and produce lactic acid as the major metabolic end product of carbohydrate fermentation.

In some embodiments of any of the aspects, 16S rRNA gene sequencing is performed on or obtained from the bacteria. 16S rRNA gene sequencing, can also be referred to as “16S ribosomal RNA sequencing”, “16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary by species. 16S rRNA gene sequencing typically comprises using a plurality of universal primers that bind to conserved regions of the 16S rRNA gene, PCR amplifying the bacterial 16S rRNA gene regions (including hypervariable regions), and sequencing the amplified 16S rRNA genes, for example, with a next-generation sequencing technology as described herein (see also e.g., U.S. Pat. Nos. 5,654,418; 6,344,316; and 8,889,358; and US Patent Application Numbers US 2013/0157265 and US 2018/0195111, which are incorporated by reference in their entireties).

In some embodiments, the bacterium or bacteria is/are selected from species comprising a 16S rRNA sequence, gene sequence, or genome sequence selected from a species listed in Table 12. In some embodiments, the bacterium or bacteria is/are selected from species comprising a 16S rRNA sequence, gene sequence, or genome sequence that is at least 90% identical to a 16S rRNA sequence, gene sequence, or genome sequence from a species listed in Table 12). In some embodiments, the bacterium or bacteria is/are selected from species comprising a 16S rRNA sequence, gene sequence, or genome sequence that is at least 80%, at least 85%, at least 90%, at least 91%, at least 92%, at least 93%, at least 94%, at least 95%, at least 96%, at least 97%, at least 98%, at least 99%, or more identical to a 16S rRNA sequence, gene sequence, or genome sequence from a species listed in Table 12. For further details about the exemplary bacterial strains listed in Table 12, see e.g., U.S. Pat. No. 11,141,443 B2, the contents of which are incorporated herein by reference in their entirety.

TABLE 12
Exemplary bacterial genome sequences

| | | Reference (available on |
|---|---|---|
| Species | ATCC Cat. | the world wide web at) |
| Lactiplantibacillus | AB101 (alias | genomes.atcc.org/genomes/ |
| plantarum | ATCC BAA- | daded83eca2b4d03 |
| | 793 ™) | |
| Lactobacillus | AB102 (alias | genomes.atcc.org/genomes/ |
| acidophilus | ATCC 4356 ™) | 4744cca046d94f76 |
| Lacticaseibacillus | AB103 (alias | genomes.atcc.org/genomes/ |
| rhamnosus | ATCC 53103 ™) | ecb332b8358541e4 |

In some embodiments, a spray-dried biotherapeutic matrix composition as described herein comprises a combination of at least two bacterial species or strains as described herein. In some embodiments, a spray-dried biotherapeutic matrix composition as described herein comprises a combination of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, or more bacterial species or strains as described herein. It is contemplated herein that such combinations can have synergistic or additive effects. In some embodiments, a spray-dried biotherapeutic matrix composition comprises 20 or fewer bacterial species or strains. In some embodiments, comprises 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or only one bacterial species or strain.

In some embodiments, the bacteria are non-pathogenic. In some embodiments of any of the aspects, the bacterium or bacteria is/are engineered to be non-pathogenic. In some embodiments of any of the aspects, the bacterium or bacteria is/are genetically modified, e.g., to produce biomolecules in the target bronchopulmonary tissue. In some embodiments of any of the aspects, the bacterium or bacteria does/do not comprise pathogenic genes. In some embodiments of any of the aspects, the bacterium or bacteria is/are engineered to inactivate at least one pathogenic gene.

In some embodiments, the spray-dried biotherapeutic matrix composition does not comprise a pathogen. As used herein, the term “pathogen” or “pathogenic” refers to any infectious microbes causing or capable of causing disease in an organism. In one embodiment, the pathogens comprise bacteria, fungi, archaea (e.g., methanogens, halophiles, thermophiles, and psychrophiles), protists (e.g., Plasmodium, Entamoeba histolytica, Trypanosoma brucei, Giardia lamblia), viruses, prions (e.g., PrPres and PrPSc), microscopic plants (e.g., Shewanella algae, Shewanella putrefaciens, and Shewanella xiamenensis), and/or microscopic animals/parasites (e.g., plankton, planarian, helminths, schistosomes, and trypanosomes). In some embodiments, the pathogenic viruses include but are not limited to RNA viruses such as flaviviruses, picornaviruses, rhabdoviruses, filoviruses, retroviruses (including lentiviruses), or DNA viruses such as adenoviruses, poxviruses, herpes viruses, cytomegaloviruses, hepadnaviruses, or others.

Non-limiting examples of pathogenic bacteria include spirochetes (e.g. Borrelia), actinomycetes (e.g. Actinomyces), mycoplasmas, Rickettsias, Gram negative aerobic rods, Gram negative aerobic cocci, Gram negative facultatively anaerobic rods (e.g. Erwinia and Yersinia), Gram-negative cocci, Gram negative coccobacilli, Gram positive cocci (e.g. Staphylococcus and Streptococcus), endospore-forming rods, and endospore-forming cocci. Further non-limiting examples of bacterial pathogens include certain species of Bacillus, Brucella, Burkholderia, Francisella, Yersinia, Streptococcus, Haemophilus, Nisseria, Listeria, Clostridium, Klebsiella, Legionella, Escherichia (e.g., E. coli), Mycobacterium, Staphylococcus, Campylobacter, Vibrio, and Salmonella, as well as drug and multidrug resistant strains and highly virulent strains of these pathogenic bacteria. Non-limiting examples of known food-borne bacterial pathogens include certain species of Salmonella, Clostridium, Campylobacter spp., Staphylococcus, Salmonella, Escherichia (e.g., E. coli), and Listeria. In some embodiments, non-limiting examples of bacterial pathogens include Bacillus anthracis, Brucella abortus, Brucella melitensis, Brucella suis, Burkholderia mallei, Burkholderia pseudomallei, Francisella tularensis, Yersinia pestis, Streptococcus Group A and B, MRSA, Streptococcus pneumonia, Haemophilus influenza, Neisseria meningitides, Listeria monocytegenes, Clostridium difficile, Klebsiella, highly virulent pathogenic strains of E. coli, Mycobacterium tuberculosis, Staphylococcus aureus, Campylobacter spp, Salmonella spp, and Clostridium perfringens, as well as drug and multidrug resistant strains and highly virulent strains of these pathogenic bacteria. In some embodiments, non-limiting examples of known food-borne bacterial pathogens include Salmonella, non typhoidal Clostridium perfringens, Campylobacter spp., Staphylococcus aureus, Salmonella, nontyphoidal, Campylobacter spp., E. coli (STEC) 0157, and Listeria monocytogenes.

In some embodiments of any of the aspects, the spray-dried biotherapeutic matrix composition is substantially free of human pathogens (e.g., as described above or known in the art). In some embodiments of any of the aspects, the spray-dried biotherapeutic matrix composition is substantially free of non-human mammal pathogens that can infect and/or cause disease in humans.

In some embodiments, the bacteria are present at a concentration of at least 10¹ colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g. In embodiments where the bacteria are non-viable, the concentration refers to the colony-forming units of the bacteria prior to killing the bacteria. In embodiments where the bacterial preparation comprises non-living components of bacteria, the concentration refers to the colony-forming units of the bacteria prior to isolation of the non-living components from the bacteria. In some embodiments, the bacteria are present at a concentration of at least 350×10⁶ colony-forming units per gram (CFU/g). In some embodiments, the bacteria are present at a concentration of at least 350×10⁹ colony-forming units per gram (CFU/g). In some embodiments, the bacteria are present at a concentration of at least 10⁶ colony-forming units per gram (CFU/g). In some embodiments, the bacteria are present at a concentration of at least 10⁸ colony-forming units per gram (CFU/g). In some embodiments, the bacteria are present at a concentration of at least 10⁹ colony-forming units per gram (CFU/g).

In some embodiments, such as those comprising viable bacteria, the bacteria are each resistant to at least one antibiotic. In some embodiments, the bacteria are collectively resistant to at least 2 antibiotics. In some embodiments, the bacteria (each on their own or collectively) are resistant to at least 1, at least 2, at least 3, at last 4, at least 5, at least 6, at least 7, at least 8, at least 9, at least 10, at least 11, at least 12, at least 13, at last 14, at least 15, at least 16, at least 17, at least 18, at least 19, at least 20 or more antibiotics. In some embodiments of any of the aspects, the antibiotic is selected from amikacin; aztreonam; cefepime; cefoxitin; ciprofloxacin; levofloxacin; metronidazole; trim/sulfa; trimethoprim; vancomycin; extended-spectrum beta-lactamases (ESBL) plazomicin; fosfomycin; ceftazidime; or ofloxacin.

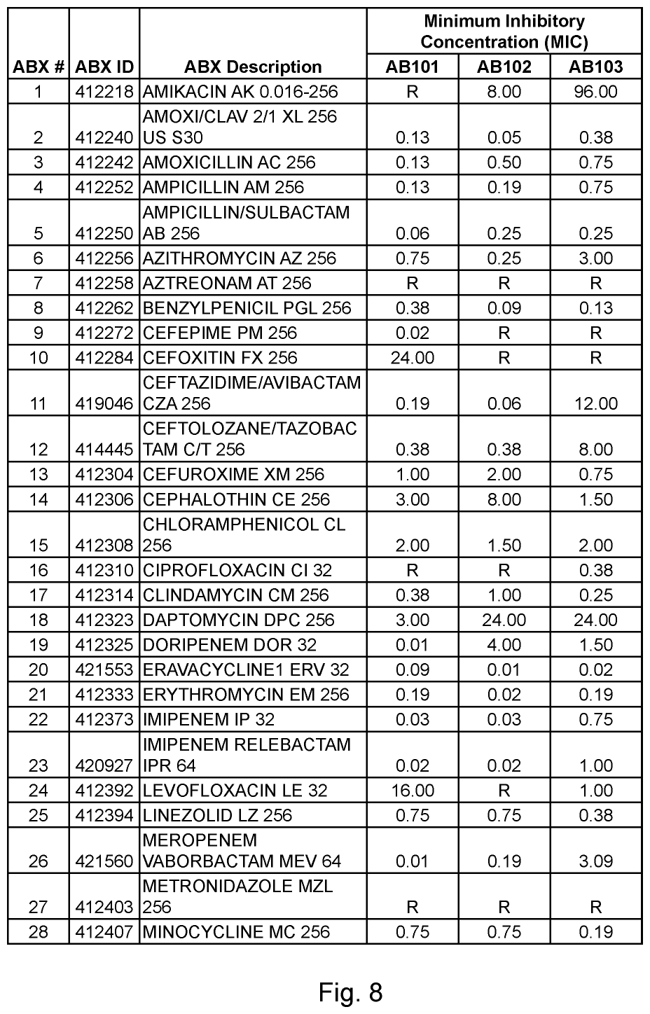

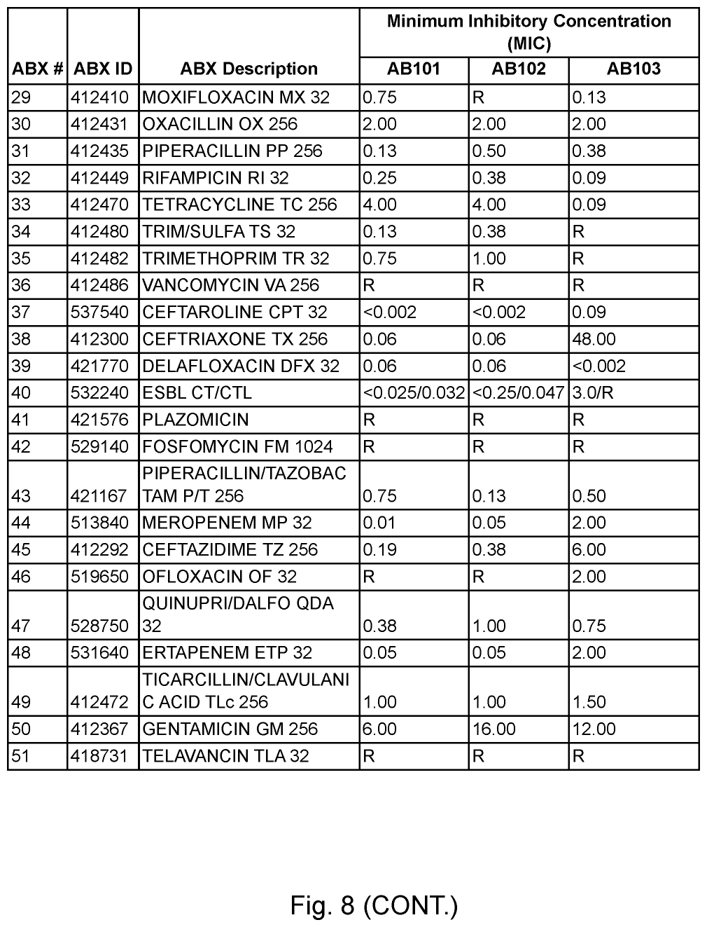

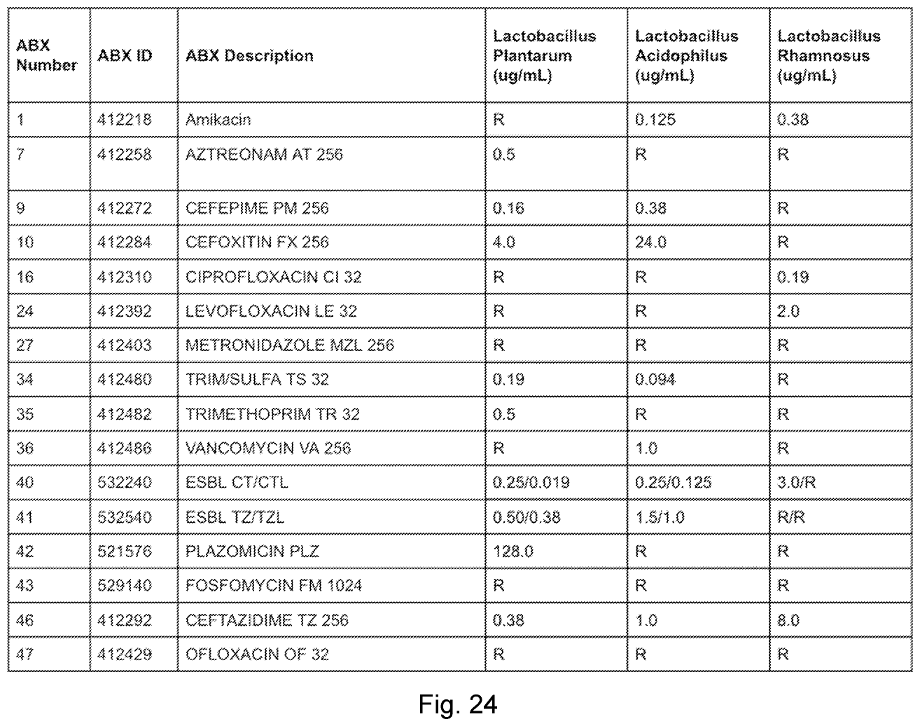

In some embodiments, such as those comprising viable bacteria, the bacteria are each susceptible to at least one antibiotic. In some embodiments, the bacteria are collectively susceptible to at least 2 antibiotics. In some embodiments, the bacteria (each on their own or collectively) are susceptible to at least 1, at least 2, at least 3, at last 4, at least 5, at least 6, at least 7, at least 8, at least 9, at least 10, at least 11, at least 12, at least 13, at last 14, at least 15, at least 16, at least 17, at least 18, at least 19, at least 20 or more antibiotics. In some embodiments of any of the aspects, the antibiotic is selected from amikacin; aztreonam; cefepime; cefoxitin; ciprofloxacin; levofloxacin; metronidazole; trim/sulfa; trimethoprim; vancomycin; extended-spectrum beta-lactamases (ESBL) plazomicin; fosfomycin; ceftazidime; or ofloxacin (see e.g., FIG. 8, FIG. 24).

In some embodiments, the spray-dried biotherapeutic matrix composition comprises at least 0.5% bacterial preparation by dry weight. In some embodiments, the spray-dried biotherapeutic matrix composition comprises at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, at least 10.0% or more bacterial preparation by dry weight.

In some embodiments, the spray-dried biotherapeutic matrix composition comprises at least 5 mg bacterial preparation per unit dose. In some embodiments, the spray-dried biotherapeutic matrix composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more bacterial preparation per unit dose.

In some embodiments, the bacterial preparation comprises a bacterial extract or bacterial metabolite preparation. In some embodiments, the bacterial extract or bacterial metabolite preparation is selected from the group consisting of: a bacterial exosome; bacterial cell wall; peptidoglycan; teichoic acid; lipoteichoic acid; bacterial S-layer; exopolysaccharide; polysaccharide; a lactic acid polymer; a lactic acid derivative; a lactic acid intermediate; hydrogen peroxide; a bacteriocin; a salivaricin; a reuterin; and a bacterial growth supernatant.

In some embodiments, the bacterial preparation comprises a bacterial exosome. Exosomes are membrane-bound extracellular vesicles (EVs) that comprises cytoplasmic, periplasmic, or transmembrane components from the bacterial cell.

In some embodiments, the bacterial preparation comprises bacterial cell wall. In some embodiments, the bacterial preparation comprises a component of the bacterial cell wall. Bacterial cell walls are made of peptidoglycan (also called murein), which is made from polysaccharide chains cross-linked by unusual peptides containing D-amino acids. In some embodiments, the bacterial preparation comprises peptidoglycan. Peptidoglycan is a polymer consisting of sugars and amino acids that forms a mesh-like peptidoglycan layer outside the plasma membrane of most bacteria, forming the cell wall. The sugar component consists of alternating residues of β-(1,4) linked N-acetylglucosamine (NAG) and N-acetylmuramic acid (NAM). Attached to the N-acetylmuramic acid is a peptide chain of three to five amino acids.

In some embodiments, the bacterial preparation comprises teichoic acid. Teichoic acids are bacterial copolymers of glycerol phosphate or ribitol phosphate and carbohydrates linked via phosphodiester bonds. Teichoic acids are found within the cell wall of most Gram-positive bacteria such as species in the genera Staphylococcus, Streptococcus, Bacillus, Clostridium, Corynebacterium, and Listeria, and can extend to the surface of the peptidoglycan layer. They can be covalently linked to N-acetylmuramic acid or a terminal D-alanine in the tetrapeptide crosslinkage between N-acetylmuramic acid units of the peptidoglycan layer, or they can be anchored in the cytoplasmic membrane with a lipid anchor. Teichoic acids that are anchored to the lipid membrane are referred to as lipoteichoic acids (LTAs), whereas teichoic acids that are covalently bound to peptidoglycan are referred to as wall teichoic acids (WTA). In some embodiments, the bacterial preparation comprises lipoteichoic acid.

In some embodiments, the bacterial preparation comprises bacterial S-layer. S-layers are two-dimensional (2D) protein arrays that are frequently found on the surface of bacteria and archaea. S-layers comprise one or more (glyco)proteins, known as S-layer proteins (SLPs), that undergo self-assembly to form a regularly spaced array on the surface of the cell. S-layers are usually composed of a single protein.

In some embodiments, the bacterial preparation comprises exopolysaccharide. Exopolysaccharides (EPS) are extracellular macromolecules excreted as tightly bound capsule or loosely attached slime layer in microorganisms. The ESP extracellular polymeric substances are natural polymers of high molecular weight secreted by microorganisms into their environment. EPSs establish the functional and structural integrity of biofilms. Exopolysaccharides generally consist of monosaccharides and some non-carbohydrate substituents (such as acetate, pyruvate, succinate, and phosphate).

In some embodiments, the bacterial preparation comprises polysaccharide, such a polysaccharide isolated from a bacterium. Bacterial polysaccharides can include capsular polysaccharides (CPSs), exopolysaccharides (EPSs), lipopolysaccharides (LPSs), teichoic acids (TAs), and peptidoglycans.

In some embodiments, the bacterial preparation comprises a lactic acid-producing compound. In some embodiments, the lactic acid-producing compound is selected from: (i) lactic acid; (ii) a non-polymeric compound that can produce lactic acid; or (iii) a polymeric compound that can produce lactic acid. In some embodiments, the non-polymeric lactic acid-producing compound is an inorganic salt of lactic acid, an ester of lactic acid, or lactide. In some embodiments, the polymeric lactic acid-producing compound is a polylactic acid (PLA). In some embodiments, the polylactic acid is poly(L-lactide) (PLLA), poly(D,L-lactide) (PDLLA), or poly(D-lactide) (PDLA). In some embodiments, the polylactic acid is poly(D,L-lactide) (PDLLA). In some embodiments, the bacterial preparation comprises a lactic acid polymer. In some embodiments, the bacterial preparation comprises a lactic acid derivative. In some embodiments, the bacterial preparation comprises a lactic acid intermediate.

In some embodiments, the bacterial preparation comprises hydrogen peroxide (H₂O₂). Hydrogen peroxide can function as a bacteriostatic agent.

In some embodiments, the bacterial preparation comprises a bacteriocin. Bacteriocins are proteinaceous or peptidic toxins produced by bacteria to inhibit the growth of similar or closely related bacterial strain(s). Non-limiting examples of bacteriocins include colicins, colicin-like bacteriocins, microcins, tailocins, Class I bacteriocins, Class II bacteriocins, Class III bacteriocins, or Class IV bacteriocins.

In some embodiments, the bacterial preparation comprises a salivaricin. Salivaricin are gene-encoded peptides that contain intramolecular ring structures (i.e., lantibiotics) first shown to be produced by Streptococcus salivarius. Non-limiting examples of salivaricins include salivaricin A, salivaricin B, salivaricin C, salivaricin D, or salivaricin E.

In some embodiments, the bacterial preparation comprises a reuterin. Reuterin (3-hydroxypropionaldehyde) is the organic compound with the formula HOCH₂CH₂CHO. The name reuterin is derived from Lactobacillus reuteri, which produces the compound biosynthetically from glycerol as a broad-spectrum antibiotic (bacteriocin).

In some embodiments, the bacterial preparation comprises a bacterial growth supernatant, which can also be referred to as a bacterial culture supernatant. A bacterial supernatant can be prepared by growing bacteria in a liquid media and then at a certain timepoint removing the liquid media; any debris or floating cells can be removed by centrifuging the media and removing the resultant liquid. A bacterial supernatant can comprise substances, such as polypeptides or peptides, secreted from the bacteria.

Bacterial preparations can be prepared by methods as known in the art. For example, live bacteria can be prepared by growing a bacterial isolate in a liquid growth medium suited to the growth of that particular bacterial species. Non-viable bacteria can be prepared by killing the bacteria, such as through heat-killing or treatment with UR light or a chemical such as alcohol. Components of bacteria can be prepared through methods such as overexpression, transformation, solvent extraction, lysis, bead-milling, centrifugation, maceration, percolation, reflux extraction, Soxhlet extraction, pressurized liquid extraction, supercritical fluid extraction, ultrasound assisted extraction, microwave assisted extraction, pulsed electric field extraction, enzyme assisted extraction, chromatography, affinity columns, immunochemistry, and the like or any combination thereof.

In some embodiments, the composition further comprises a prebiotic (such as, but not limited to, amino acids (e.g., arginine, glutarate, and ornithine), biotin, fructooligosaccharide, galactooligosaccharides, hemi celluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) xylooligosaccharides, polyamines (such as but not limited to spermidine and putrescine).

In some embodiments, the bacterial preparation comprises at least one bacterial species (or extract or metabolite thereof) and at least one of the following: a filler/binder; a cryoprotectant; ascorbic acid; and/or an oligosaccharide (see e.g., Table 1C). A non-limiting example of a filler or binder includes GLUCIDEX (e.g., Maize maltodextrin II). Non-limiting examples of a cryoprotectant include: trehalose or sodium glutamate. Non-limiting examples of an oligosaccharide include betacyclodextrin or sucrose.

In some embodiments, the bacterial preparation (see e.g., Table 1C) is freeze-dried prior to being used to prepare the solid and/or liquid feedstocks described herein; in such embodiments, the bacteria are freeze-dried and then spray-dried during the preparation of the spray-dried biotherapeutic matrix composition described herein. In some embodiments, the bacterial preparation is spray-dried prior to being used to prepare the solid and/or liquid feedstocks described herein; in such embodiments, the bacteria are spray-dried twice during the preparation of the spray-dried biotherapeutic matrix composition described herein.

In some embodiments, the bacterial preparation comprises fresh, non-spray dried bacteria that is added directly to the solid and/or liquid feedstocks described herein; in such embodiments, the bacteria are spray-dried once during the preparation of the spray-dried biotherapeutic matrix composition described herein. In some embodiments, the bacteria are fermented prior to being used to prepare the solid and/or liquid feedstocks described herein.

An exemplary procedure for preparing a bacterial preparation includes: (a) seeding a bacterial inoculum (e.g., from a working cell bank) into a liquid growth medium, (b) fermenting the bacteria in the liquid growth medium, (c) harvesting the bacteria from the liquid growth medium (e.g., centrifugation), and (d) freeze-drying the harvested bacteria. This freeze-drying process to prepare the bacterial preparation can include at least one of the excipient(s) and/or at least one of the stabilizer(s) described herein; additional amounts of the at least one excipient(s) and/or the at least one stabilizer(s) can be added to prepare the spray-dried biotherapeutic matrix composition (see e.g., Tables 1A-1C).

### Pharmaceutically Acceptable Excipients, Stabilizers, and Additives

In some embodiments, the technology described herein relates to a spray-dried biotherapeutic matrix composition comprising a bacterial preparation as described herein, and optionally a pharmaceutically acceptable excipient, stabilizer, and/or additive. In some embodiments, the active ingredients of the spray-dried biotherapeutic matrix composition comprise bacterial preparation as described herein. In some embodiments, the active ingredients of the spray-dried biotherapeutic matrix composition consist essentially of bacterial preparation as described herein. In some embodiments, the active ingredients of the spray-dried biotherapeutic matrix composition consist of bacterial preparation as described herein. In some embodiments, pharmaceutically acceptable excipients, stabilizers, and/or additives include saline, aqueous buffer solutions, solvents and/or dispersion media. The use of such excipients, stabilizers, and/or additives is well known in the art. Some non-limiting examples of materials which can serve as pharmaceutically-acceptable excipients, stabilizers, and/or additives include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. In some embodiments, the pharmaceutically acceptable excipient, stabilizer, and/or additive inhibits the degradation of the active agent, e.g. a bacterial preparation as described herein. In some embodiments, the spray-dried biotherapeutic matrix composition comprises at least one pharmaceutically acceptable excipient and/or at least one pharmaceutically acceptable stabilizer.

### Excipients

In some embodiments, the spray-dried biotherapeutic matrix composition at least one excipient. In some embodiments, the spray-dried biotherapeutic matrix composition comprises at least 1, 2, 3, 4, 5, 6, 7, 8, 9, 10, or more excipients. As used herein, the term “excipient” refers to an inactive substance that serves as the vehicle, diluent, or medium for the active substance, e.g., the bacterial preparation as described herein. Excipients can ease processability of the spray-dried biotherapeutic matrix composition (e.g., formulated for respiratory administration) and maintain physical structure of the composition for added long term stability. Excipients can provide stability, protect viability (e.g., of live biotherapeutics), and enhance aerosol characteristics of the spray-dried biotherapeutic matrix composition. In some embodiments, the composition comprises at least two excipients. In some embodiments, the composition comprises two excipients. In some embodiments, the composition comprises three excipients. In some embodiments, the composition comprises at least 2, at least 3, at least 4, at least 5, at least 6, at least 7, at least 8, at least 9, at least 10, or more excipients.

Typically, excipients are used when spray drying pharmaceutical drug products for two purposes: adding dispersibility to the final powder, and glass stabilization of crystalline or amorphous particles with high viscosity; see e.g., Vehring (2008) Pharm Res 25 (5):999-1022; the content of which is incorporated herein by reference in its entirety. Amino acids (e.g., leucine and trileucine) can be used for the effect of adding dispersibility to the final powder. A dispersible powder tends to clump together less readily, and tends to be more flowable in bulk, making encapsulation easier.

As spray drying creates a particle in an energetically unfavorable state, excipients used for glass stabilization are helpful in providing longer-term stability advantages and reliable solid-state chemical features, such as stable crystallinity or co-crystallinity leading to consistent bioavailability in vivo. The effectiveness of these excipients is typically evaluated by measuring the glass transition temperature of the dried particle, which is best maintained well above storage temperature (e.g., above −18° C. freezer, 4° C. refrigeration, or 20-22° C. room temperature) to ensure physical stability. In some embodiments, the minimum Tg (glass transition temperature) is 35° C. In some embodiments, the Tg (glass transition temperature) is at least 30° C., at least 35° C., at least 40° C., at least 45° C., or at least 50° C.

In some embodiments, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; camitine; acetylcamitine; propionylcamitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

In some embodiments, the excipient is selected from Table 2. In some embodiments, the excipient is selected from any combination of excipients listed in Table 2, e.g., a combination of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39 excipients from Table 2.

TABLE 2
Exemplary Excipients

| | | Glycine | |
|---|---|---|---|
| Excipients | Inulin | betaine | Starch |
| MRS growth medium | Sodium caseinate | Glycogen | Lactose |
| Gelatin | Goat's milk | Trehalose | Sucrose |
| Whey Isolate | Cow's milk | Mannose | Glucose |
| Sweet Whey | Proline | Xylose | Leucine |
| | | | and/or |
| | | | Trileucine |
| Reconstituted | Carnitine | Mannitol | Sodium salts |
| skim milk | | | |
| Maltodextrins | Acetylcarnitine | Sorbitol | Potassium |
| | | | salts |
| Gluco- | Propionylcarnitine | Maltose | Lithium |
| oligosaccharides | | | salts |
| Lacto- | Glutamate | Dextrose | Calcium |
| oligosaccharides | | | salts |
| Fructo- | Sodium citrate | | |
| oligosaccharides | | | |

In some embodiments, the excipient is leucine, trehalose, and/or sodium citrate. In some embodiments, the excipient is leucine and/or trehalose. In some embodiments, the excipient is leucine and/or sodium citrate. In some embodiments, the excipient is trehalose and/or sodium citrate.

In some embodiments, the excipient is leucine. In some embodiments, the excipient is L-leucine (see e.g., Formula I, below). In some embodiments, the excipient is D-leucine. In some embodiments, the excipient is a racemic mixture of L-leucine and D-leucine. As used, here “racemic mixture” refers to a solution in which there is 50:50 ratio of both enantiomers of a compound. Leucine (symbol Leu or L) is an essential amino acid for humans. In some embodiments, the excipient is trileucine (also referred to as Leu-Leu-Leu), which is a tripeptide composed of three leucine residues (see e.g., Formula II, below). In some embodiments, trileucine is used in place of leucine as an excipient. In some embodiments, the excipient is trehalose (see e.g., Formula III, below). Trehalose is a sugar consisting of two molecules of glucose. Trehalose is also known as mycose or tremalose. Trehalose has high water retention capabilities. Some bacteria, fungi, plants and invertebrate animals synthesize trehalose as a source of energy, and to survive freezing and lack of water. In some embodiments, the excipient is leucine and trehalose. In some embodiments, the first excipient is leucine and the second excipient is trehalose. In some embodiments, the second excipient is leucine and the first excipient is trehalose. In some embodiments, the excipient is sodium citrate (Na₃C₆H₅O₇; see e.g., Formula IV, below) In some embodiments, the first and second excipients are leucine and trehalose, and the third excipient is sodium citrate. In some embodiments, the three excipients are leucine, trehalose, and sodium citrate, and the stabilizer is polysorbate 80.

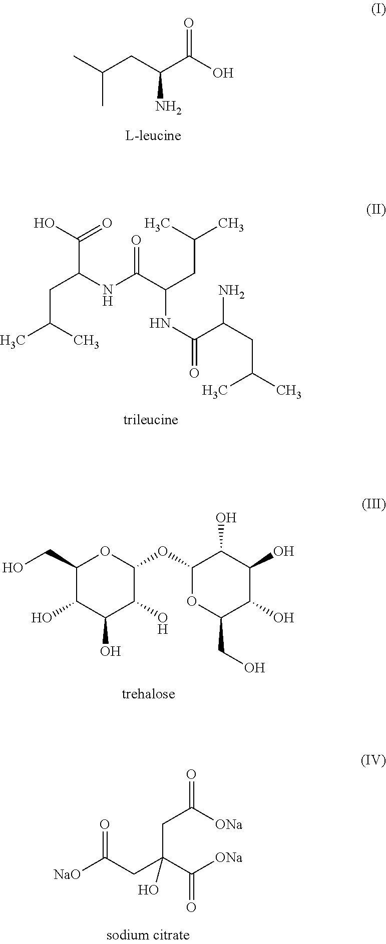

In some embodiments, the spray-dried biotherapeutic matrix composition comprises at least 0.5% excipient by weight. In some embodiments, the spray-dried biotherapeutic matrix composition comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 10%, at least 15%, at least 20%, at least 25%, or at least 30% or more excipient by weight.

In some embodiments, the spray-dried biotherapeutic matrix composition comprises at least 0.5% of a first excipient by weight, and at least 0.5% of a second excipient by weight. In some embodiments, the spray-dried biotherapeutic matrix composition comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, or at least 5.0% or more of a first excipient by weight, and at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, or at least 5.0% or more of a second excipient by weight.

In some embodiments, the spray-dried biotherapeutic matrix composition comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, or at least 5.0% or more of a first excipient by weight, and at least 0.5% of a second excipient by weight. In some embodiments, the spray-dried biotherapeutic matrix composition comprises at least 0.5% of a first excipient by weight, and at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, or at least 5.0% or more of a second excipient by weight.

In some embodiments, the spray-dried biotherapeutic matrix composition comprises at least 5 mg excipient per unit dose. In some embodiments, the spray-dried biotherapeutic matrix composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more excipient per unit dose.

In some embodiments, the spray-dried biotherapeutic matrix composition comprises at least 5 mg of a first excipient, and at least 5 mg of a second excipient per unit dose. In some embodiments, the spray-dried biotherapeutic matrix composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a first excipient, and at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a second excipient per unit dose.

In some embodiments, the spray-dried biotherapeutic matrix composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a first excipient, and at least 5 mg of a second excipient per unit dose.

In some embodiments, the spray-dried biotherapeutic matrix composition comprises at least 5 mg of a first excipient, and at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a second excipient per unit dose.

In some embodiments, the spray-dried biotherapeutic matrix composition comprises at least 5 mg of a first excipient, at least 5 mg of a second excipient, and at least 1 mg of a third excipient per unit dose. In some embodiments, the spray-dried biotherapeutic matrix composition comprises at least 5 mg of a first excipient, at least 5 mg of a second excipient, and at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a third excipient per unit dose.

### Stabilizers

In some embodiments, the spray-dried biotherapeutic matrix composition comprises at least one stabilizer. In some embodiments, the spray-dried biotherapeutic matrix composition comprises at least 1, 2, 3, 4, 5, 6, 7, 8, 9, 10, or more stabilizers. As used herein, the term “stabilizer” refers to a substance which prevents or reduces the breakdown of the spray-dried biotherapeutic matrix composition. A surfactant stabilizer can provide initial wettability during feedstock preparation, emulsification of the feedstock during spray drying, and solid-state protection of the dry powder through surface water protection of the therapeutic agent.

The use of stabilizers aids the process of wetting a hydrophobic substance to create a homogeneous suspension for spray drying. For example, a stabilizer can be included in case a wetting agent is needed to get any poorly soluble compounds into suspension. These stabilizers act as an emulsifier to enhance consistency across the bulk spray dried powder. In addition, some stabilizers (e.g., Poloxamers) can be used to pre-disperse polymers in feedstock solutions, even allowing for tunable wetted particle sizing down to the nanoscale; see e.g., Da Silva et al. (2019) Front Bioeng Biotechnol 7:137; the content of which is incorporated herein by reference in its entirety.

In some embodiments, the stabilizer comprises a surfactant. A surfactant is a substance which tends to reduce the surface tension of a liquid in which it is dissolved. In some embodiments, the stabilizer is selected from the group consisting of: mannitol, carboxymethyl cellulose (CMC), polyvinyl alcohol (PVA), polysorbate, and poloxamer. In some embodiments, the stabilizer is a polysorbate, poloxamer, or polyvinyl alcohol.

In some embodiments, the stabilizer is a Polysorbate. Polysorbate is a synthetic nonionic surfactant and emulsifier. Polysorbate is also referred to as MONTANOX; ALKEST TW; TWEEN; or PS. In some embodiments, the stabilizer is Polysorbate 20, Polysorbate 40, Polysorbate 60, or Polysorbate 80. The number following the ‘polysorbate’ part is related to the type of fatty acid associated with the polyoxyethylene sorbitan part of the molecule. Monolaurate is indicated by 20 (e.g., Polysorbate 20), monopalmitate is indicated by 40 (e.g., Polysorbate 40), monostearate by 60 (e.g., Polysorbate 60), and monooleate by 80 (e.g., Polysorbate 80).

In some embodiments, the stabilizer is Polysorbate 80 (see e.g., Formula V, below). Polysorbate 80 is also referred to as Polyoxyethylene (20) sorbitan monooleate (number 20 following the ‘polyoxyethylene’ part refers to the total number of oxyethylene —(CH₂CH₂O)—groups found in the molecule); (x)-sorbitan mono-9-octadecenoate poly(oxy-1,2-ethanediyl); MONTANOX 80; ALKEST TW 80; TWEEN 80; or PS 80. Polysorbate 80 is derived from polyethoxylated sorbitan and oleic acid. The hydrophilic groups in this compound are polyethers also known as polyoxyethylene groups, which are polymers of ethylene oxide. In the nomenclature of polysorbates, the numeric designation following polysorbate refers to the lipophilic group, in this case, the oleic acid. The critical micelle concentration of polysorbate 80 in pure water is reported as 0.012 mM.

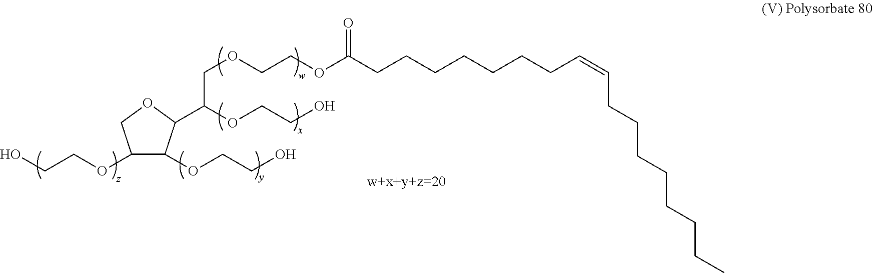

In some embodiments, the stabilizer is a poloxamer. Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are also referred to as PLURONIC, KOLLIPHOR, or SYNPERONIC. Because the lengths of the polymer blocks can be customized, many different poloxamers exist that have slightly different properties. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mo, and a 70% polyoxyethylene content). For the PLURONIC and SYNPERONIC names, coding of these copolymers starts with a letter to define its physical form at room temperature (L=liquid, P=paste, F=flake (solid)) followed by two or three digits, The first digit (or two digits in a three-digit number) in the numerical designation, multiplied by 300, indicates the approximate molecular weight of the hydrophobe; and the last digit multiplied by 10 gives the percentage polyoxyethylene content (e.g., L61 indicates a polyoxypropylene molecular mass of 1800 g/mol and a 10% polyoxyethylene content). In the example given, poloxamer 181 (P181)=PLURONIC L61 and SYNPERONIC PE/L 61.

One characteristic of poloxamer solutions is their temperature dependent self-assembling and thermo-gelling behavior. Concentrated aqueous solutions of poloxamers are liquid at low temperature and form a gel at higher temperature in a reversible process. The transitions that occur in these systems depend on the polymer composition. Because of their amphiphilic structures, poloxamers have surfactant properties. Among other things, poloxamers can be used to increase the water solubility of hydrophobic, oily substances or otherwise increase the miscibility of two substances with different hydrophobicities. See e.g., Table 3 for physicochemical properties of exemplary poloxamers.

TABLE 3
Physicochemical properties of PLURONIC copolymers.

| | | | | cmc at | cmc at | cmc at |
|---|---|---|---|---|---|---|
| Pluronic ® | | PO | EO | 25° C. | 30° C. | 35° C. |
| Notation | MW | Units | Units | (% w/v) | (% w/v) | (% w/v) |
| L64 | 2900 | 30 | 26 | n/a | 1.5 | 0.4 |
| P65 | 3400 | 17 | 36 | n/a | 4 | 1 |
| P84 | 4200 | 43 | 38 | 2.6 | 0.6 | 0.15 |
| P85 | 4600 | 40 | 52 | 4 | 0.9 | 0.2 |
| F88 | 11,400 | 39 | 206 | n/a | n/a | 1.7 |
| P103 | 4950 | 60 | 34 | 0.07 | 0.01 | 0.002 |
| P104 | 5900 | 61 | 54 | 0.3 | 0.04 | 0.008 |
| P105 | 6500 | 56 | 74 | 0.3 | 0.025 | 0.005 |
| F108 | 14,600 | 50 | 264 | 4.5 | 0.8 | 0.15 |
| P123 | 5750 | 69 | 38 | 0.03 | 0.005 | 0.001 |
| F127 | 12,600 | 65 | 200 | 0.7 | 0.1 | 0.025 |

MW, molecular weight;
PO, propylene oxide;
EO, ethylene oxide;
cmc, critical micellization concentration;
L, liquid;
P, paste;
F, flake.
See e.g., Bodratti and Alexandridis, J Funct Biomater, 2018; 9(1): 11; the content of which is incorporated herein by reference in its entirety.

In some embodiments, the stabilizer is Poloxamer 184 (i.e., PLURONIC L64), Poloxamer 185 (i.e., PLURONIC P65), Poloxamer 188 (i.e., PLURONIC F68), Poloxamer 234 (i.e., PLURONIC P84), Poloxamer 235 (i.e., PLURONIC P85), Poloxamer 238 (i.e., PLURONIC F88), Poloxamer 333 (i.e., PLURONIC P103), Poloxamer 334 (i.e., PLURONIC P104), Poloxamer 335 (i.e., PLURONIC P105), Poloxamer 338 (i.e., PLURONIC F108), Poloxamer 403 (i.e., PLURONIC P123), or Poloxamer 407 (i.e., PLURONIC F127). In some embodiments, the stabilizer is Poloxamer 184, Poloxamer 185, Poloxamer 234, Poloxamer 235, Poloxamer 238, Poloxamer 333, Poloxamer 334, Poloxamer 335, Poloxamer 338, Poloxamer 403, or Poloxamer 407. In some embodiments, the stabilizer is PLURONIC L64, PLURONIC P65, PLURONIC P84, PLURONIC P85, PLURONIC F88, PLURONIC P103, PLURONIC P104, PLURONIC P105, PLURONIC F108, PLURONIC P123, or PLURONIC F127.

In some embodiments, the stabilizer is poloxamer 188, also referred to as PLURONIC F68 or Polyoxyethylene-polyoxypropylene block copolymer (linear formula: (C₃H₆O·C₂H₄O)ₓ; see e.g., Formula VI, below). In some embodiments, the spray-dried biotherapeutic matrix composition comprises Polysorbate 80. In some embodiments, the spray-dried biotherapeutic matrix composition comprises Poloxamer 188 (i.e., PLURONIC F68). In some embodiments, the spray-dried biotherapeutic matrix composition comprises Polysorbate 80 and Poloxamer 188 (i.e., PLURONIC F68).

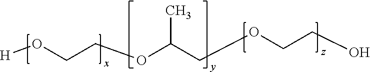

In some embodiments, the stabilizer is polyvinyl alcohol (PVA; see e.g., Formula VII, below)). PVA is a water-soluble synthetic polymer, with the idealized formula [CH₂CH(OH)]ₙ. PVA can be used as a thickener and emulsion stabilizer. PVA exhibits biocompatibility, a low tendency for protein adhesion, and low toxicity. PVA is prepared by hydrolysis of polyvinyl acetate, or other vinyl ester-derived polymers with formate or chloroacetate groups instead of acetate. The conversion of the polyvinyl esters is can be conducted by base-catalyzed transesterification with ethanol.

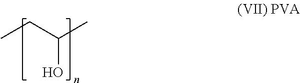

In some embodiments, the spray-dried biotherapeutic matrix composition comprises at least 0.05% stabilizer by weight. In some embodiments of any of the aspects, the composition comprises at least 0.25% of the stabilizer or stabilizers by weight. In some embodiments of any of the aspects, the composition comprises at least 0.5% of the stabilizer or stabilizers by weight (e.g., dry weight). In some embodiments, the spray-dried biotherapeutic matrix composition comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0% or more stabilizer by weight.

In some embodiments, the spray-dried biotherapeutic matrix composition comprises at least 1 mg stabilizer per unit dose. In some embodiments, the spray-dried biotherapeutic matrix composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, or at least 10 mg stabilizer per unit dose.

In some embodiments, the spray-dried biotherapeutic matrix composition comprises at least one excipient and at least one stabilizer. In some embodiments, the spray-dried biotherapeutic matrix composition comprises one excipient and one stabilizer. In some embodiments, the spray-dried biotherapeutic matrix composition comprises at least two excipients and at least one stabilizer. In some embodiments, the spray-dried biotherapeutic matrix composition comprises two excipients and one stabilizer. In some embodiments, the spray-dried biotherapeutic matrix composition comprises leucine and trehalose as excipients and Poloxamer 188 as a stabilizer. In some embodiments, the spray-dried biotherapeutic matrix composition comprises leucine and trehalose as excipients and Polysorbate 80 as a stabilizer.

### Additives

In some embodiments, the spray-dried biotherapeutic matrix composition further comprises at least one additive. In some embodiments, the spray-dried biotherapeutic matrix composition comprises at least 1, 2, 3, 4, 5, 6, 7, 8, 9, 10, or more additives. As described herein, a number of additives can be added to the polymeric particles—these additives can be incorporated by a mixing at a molecule level, a dry blend, a coating onto the particles, or co-administered. In some embodiments, the spray-dried biotherapeutic matrix composition further comprises at least one of the following: (a) a pore-forming agent; (b) an adhesion agent; and/or (c) a pH-modulating agent.

In some embodiments, the spray-dried biotherapeutic matrix composition further comprises a pore-forming agent. Pore forming agents can decrease the density of the particle (i.e., by forming air spaces or “pores” in the particle) and allow for more rapid water uptake, degradation, and acid production. In some embodiments, the pore-forming agent is selected from the group consisting of: NaCl, sucrose, polyethylene glycol (PEG), and polyvinylpyrrolidone (PVP).

In some embodiments, the spray-dried biotherapeutic matrix composition further comprises an adhesion agent. Adhesion agents can increase bio-adhesion of the polymer to biological tissue. In some embodiments, the adhesion agent is selected from the group consisting of: sugars, adhesive polymer, and amine-containing compounds. Non-limiting examples of such adhesive sugars include trehalose, mannitol, lactose, or glucose.

In some embodiments, the spray-dried biotherapeutic matrix composition further comprises a pH-modulating agent. pH impacting agents can increase or decrease bacterial preparation degradation. In some embodiments, the pH-modulating agent is a buffer, such as sodium citrate. In some embodiments, the pH-modulating agent is an acid. In some embodiments, the pH-modulating agent is base (e.g., NaOH); such a base can be a neutralizing agent when creating feedstocks to spray dry for inhaled powders.

In some embodiments, the spray-dried biotherapeutic matrix composition comprises at least one additional therapeutic, e.g., for a chronic or infectious bronchopulmonary disorder. A range of drug classes include anti-inflammatories, antimicrobials, antivirals, antifungals, vasodilators, and bronchodilators. These drugs can be incorporated using techniques such as microencapsulation, co-administration, or covalently linking with a degradable linker.

In some embodiments, the spray-dried biotherapeutic matrix composition comprises an anti-inflammatory. In some embodiments, the anti-inflammatory is selected from the group consisting of: non-steroidal anti-inflammatory drugs (NSAIDs); corticosteroids; glucocorticoids; methotrexate; sulfasalazine; leflunomide; anti-tumor necrosis factor (TNF) medications; cyclophosphamide; pro-resolving lipid mediators; mycophenolate; opiates; and barbiturates.

In some embodiments, the spray-dried biotherapeutic matrix composition comprises an antimicrobial, an antiviral, and/or an antifungal. In some embodiments, the antimicrobial is selected from the group consisting of: aminoglycosides; ansamycins; beta-lactams; bis-biguanides; carbacephems; carbapenems; cationic polypeptides; cephalosporins; fluoroquinolones; glycopeptides; iron-sequestering glycoproteins; linosamides; lipopeptides; macrolides; monobactams; nitrofurans; oxazolidinones; penicillins; polypeptides; quaternary ammonium compounds; quinolones; silver compounds; sulfonamides; and tetracyclines.

Some exemplary specific antimicrobial agents include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefietazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

Non-limiting examples of antivirals include Abacavir, Acyclovir, Adefovir, Amantadine, Ampligen, Amprenavir, antiretroviral, Arbidol, Atazanavir, Atripla, Cidofovir, Combivir, Darunavir, Delavirdine, Didanosine, Docosanol, Dolutegravir, Ecoliever, Edoxudine, Efavirenz, Emtricitabine, Enfuvirtide, Entecavir, Famciclovir, Fomivirsen, Fosamprenavir, Foscamet, Fosfonet, Fusion inhibitor, Ibacitabine, Idoxuridine, Imiquimod, Imunovir, Indinavir, Inosine, Integrase inhibitor, Interferon, Interferon type I, Interferon type II, Interferon type III, Lamivudine, Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Penciclovir, Peramivir, Pleconaril, Podophyllotoxin, viral protease inhibitor, Pyramidine, Raltegravir, Reverse transcriptase inhibitor, Ribavirin, Rimantadine, Ritonavir, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), or Zidovudine.

Non-limiting examples of anti-fungals (also referred to as antimycotics) include polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles, Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur.

In some embodiments, the spray-dried biotherapeutic matrix composition comprises at least one bacteriophage. Non-limiting examples of bacteriophage include: Caudovirales such as Myoviridae, Siphoviridae and Podiviridae; Ligamenvirales such as Lipothrixviridae, Rudiviridae, Ampullaviridae, Bicaudaviridae, Clavaviridae, Corticoviridae, Cystoviridae and Fuselloviridae; and other families such as Globuloviridae, Inoviridae, Leviviridae, Microviridae, Plasmaviridae and Techtiviridae.

In some embodiments, the spray-dried biotherapeutic matrix composition comprises a vasodilator. In some embodiments, the vasodilator is selected from the group consisting of: an angiotensin converting enzyme (ACE) inhibitor; an angiotensin receptor blocker (ARB); a calcium channel blocker (CCB); and a nitric-oxide-producing compound.

In some embodiments, the vasodilator is an ACE inhibitor selected from the group consisting of: benazepril (LOTENSIN); captopril (CAPOTEN); enalapril (VASOTEC, EPANED); fosinopril (MONOPRIL); lisinopril (PRINIVIL, ZESTRIL); moexipril (UNIVASC); perindopril (ACEON); quinapril (ACCUPRIL); ramipril (ALTACE); and trandolapril (MAVIK).

In some embodiments, the vasodilator is an ARB selected from the group consisting of: azilsartan (EDARBI); candesartan (ATACAND); eprosartan (TEVETEN); irbesartan (AVAPRO); telmisartan (MICARDIS); valsartan (DIOVAN); losartan (COZAAR); and olmesartan (BENICAR).

In some embodiments, the vasodilator is a CCB selected from the group consisting of: amlodipine (NORVASC); clevidipine (CLEVIPREX); diltiazem (CARDIZEM); felodipine (CARDENE, CARDENE SR); isradipine; nicardipine; nimodipine; nisoldipine (SULAR); and verapamil (CALAN).

In some embodiments, the vasodilator is a nitric-oxide-producing compound selected from the group consisting of: nitroglycerin (GONITRO, NITROBID, NITROMIST, NITROLINGUAL, NITROSTAT, NITROBID); isosorbide mononitrate (ISMO, MONEKET); isosorbide dinitrate (IMDUR, ISORDIL); hydralazine (APRESOLINE); minoxidil; fenoldopam (CARLOPAM); and nitroprusside (NIPRIDE, NITROPRESS).

In some embodiments, the spray-dried biotherapeutic matrix composition further comprises a bronchodilator as an additional therapeutic. In some embodiments, the bronchodilator is selected from the group consisting of: a short-acting beta-adrenergic bronchodilator (e.g., albuterol, levalbuterol, epinephrine); a long-acting beta-adrenergic bronchodilator (e.g., salmeterol, formoterol); an anticholinergic bronchodilator (e.g., ipratropium, tiotropium); and a xanthine derivative (e.g., theophylline, aminophylline). In some embodiments of any of the aspects, the bronchodilator is albuterol.

In some embodiments, the at least one additional therapeutic is microencapsulated. In some embodiments, the additional therapeutic is microencapsulated in smaller capsules within the entire spray-dried biotherapeutic matrix composition (i.e., physically separate from the bacterial preparation). In some embodiments, the spray-dried biotherapeutic matrix composition comprises separate particles or microspheres for the therapeutics and the bacterial preparation, e.g., that are mixed together during administration. In some embodiments, the additional therapeutic is microencapsulated together with the bacterial preparation.

In some embodiments, the at least one additional therapeutic is covalently linked with a biodegradable, degradable, cleavable, or otherwise reversible linker to a component in the bacterial preparation. A cleavable linker means that the linker can be cleaved to release the two parts the linker is holding together. In some embodiments, the cleavable linker is polyethylene glycol. A cleavable linker can be susceptible to cleavage agents, such as, but not limited to, enzymes, pH, redox potential, or the presence of degradative molecules. Examples of such cleavage agents include: redox agents which are selected for particular substrates or which have no substrate specificity, including, e.g., oxidative or reductive enzymes or reductive agents, such as mercaptans, present in cells that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific), proteases, and phosphatases.

In some embodiments, the spray-dried biotherapeutic matrix composition is co-administered with the at least one additional therapeutic, e.g., for a chronic bronchopulmonary disorder. In some embodiments, the spray-dried biotherapeutic matrix composition is administered at the same time as the at least one additional therapeutic, e.g., for a chronic bronchopulmonary disorder. In some embodiments, the spray-dried biotherapeutic matrix composition is administered before the at least one additional therapeutic, e.g., for a chronic bronchopulmonary disorder. In some embodiments, the spray-dried biotherapeutic matrix composition is administered after at least one additional therapeutic, e.g., for a chronic bronchopulmonary disorder. In some embodiments, the spray-dried biotherapeutic matrix composition is administered in alternating times with the at least one additional therapeutic, e.g., for a chronic bronchopulmonary disorder.

### Formulation

In some embodiments, the spray-dried biotherapeutic matrix composition is formulated for administration by inhalation. As used herein, the term “formulated for administration by inhalation” means that a composition or formulation is designed or adapted to deliver an active ingredient or agent to bronchopulmonary tissue by inhalation of small particles or droplets containing the active ingredient. As such, a pharmaceutical composition formulated for administration by inhalation generally comprises such particles (e.g., as a powder) or can readily generate such droplets (e.g., via a nebulizer). In some embodiments, the spray-dried biotherapeutic matrix composition is formulated for delivery to the trachea, the bronchi, the bronchioles, and/or the alveoli. In some embodiments, the spray-dried biotherapeutic matrix composition is formulated for delivery to the trachea. In some embodiments, the spray-dried biotherapeutic matrix composition is formulated for delivery to the bronchi. In some embodiments, the spray-dried biotherapeutic matrix composition is formulated for delivery to the bronchioles. In some embodiments, the spray-dried biotherapeutic matrix composition is formulated for delivery to the alveoli. In some embodiments, the composition is formulated for delivery to the lungs.

In some embodiments, the composition is formulated as a capsule or a tablet for administration by inhalation (e.g., using an inhaler). In some embodiments, the capsule contains at least 10 mg of the spray-dried biotherapeutic matrix composition. In some embodiments, the capsule contains at least 30 mg of the spray-dried biotherapeutic matrix composition. In some embodiments, the capsule contains at least 10 mg, at least 15 mg, at least 20 mg, at least 25 mg, at least 30 mg, at least 40 mg, at least 50 mg, at least 60 mg, at least 70 mg, at least 80 mg at least 90 mg, or at least 100 mg or more of the spray-dried biotherapeutic matrix composition.

In some embodiments, the composition is formulated for delivery by an inhaler. In some embodiments, the composition is formulated for delivery by a metered dose inhaler (MDI). A metered-dose inhaler (MDI) is a device that delivers a specific amount of medication to the lungs, in the form of a short burst of aerosolized medicine that is usually self-administered by the patient via inhalation. In some embodiments, the composition is formulated for delivery by a dry powder inhaler (DPI). A dry-powder inhaler (DPI) is a device that delivers medication to the lungs in the form of a dry powder. In some embodiments, the composition is formulated for delivery by a soft mist inhaler (SMI). A soft mist inhaler (SMI) is a device that delivers medication to the lungs in the form of a mist.

In some embodiments, the spray-dried biotherapeutic matrix composition comprises at least 10⁶ CFU of the bacterial preparation per unit dose that is deliverable to a target tissue. In some embodiments, the spray-dried biotherapeutic matrix composition comprises at least 200×10⁶ CFU of the bacterial preparation per unit dose that is deliverable to a target tissue. In some embodiments, the spray-dried biotherapeutic matrix composition comprises at least 10¹ CFU, at least 10² CFU, at least 10³ CFU, at least 10⁴ CFU, at least 10⁵ CFU, at least 10⁶ CFU, at least 10⁷ CFU, at least 10⁸ CFU, at least 10⁹ CFU, at least 10¹⁰ CFU, at least 10¹¹ CFU, or at least 10¹² CFU of the bacterial preparation per unit dose that is deliverable to a target tissue.

In some embodiments, the spray-dried biotherapeutic matrix composition comprises at least 5 mg of the bacterial preparation per unit dose that is deliverable to a target tissue. In some embodiments, the spray-dried biotherapeutic matrix composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of the bacterial preparation per unit dose that is deliverable to a target tissue.

In some embodiments, the spray-dried biotherapeutic matrix composition comprises at least 15 mg of the bacterial preparation per unit dose. In some embodiments, the spray-dried biotherapeutic matrix composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of the bacterial preparation per unit dose.

In some embodiments, spray-dried biotherapeutic matrix composition is formulated as microspheres. In some embodiments, the microspheres have a median mass aerodynamic diameter (MMAD) of at least 1 μm to at most 1 mm. In some embodiments, the microspheres have a median mass aerodynamic diameter (MMAD) of at least 1 μm, at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, at least 900 μm, or at least 1 mm. In some embodiments, the microspheres have a median mass aerodynamic diameter (MMAD) of at most 1 μm, at most 2 μm, at most 3 μm, at most 4 μm, at most 5 μm, at most 6 μm, at most 7 μm, at most 8 μm, at most 9 μm, at most 10 μm, at most 20 μm, at most 30 μm, at most 40 μm, at most 50 μm, at most 60 μm, at most 70 μm, at most 80 μm, at most 90 μm, at most 100 μm, at most 200 μm, at most 300 μm, at most 400 μm, at most 500 μm, at most 600 μm, at most 700 μm, at most 800 μm, at most 900 μm, or at most 1 mm.

### Dried Particles

In some embodiments, the spray-dried biotherapeutic matrix composition comprises a plurality of dried particles, including multiple dried particles of the same type and/or multiple types of dried particles (e.g., as defined exemplary particle characteristics in Table 4A-4B or Table 5A-5B). In some embodiments, the spray-dried biotherapeutic matrix composition is a dry powder comprising particles with the physical characteristics described in Table 4A-4B3 or Table 5A-51B.

TABLE 4A
Exemplary Particle Characteristics

| | Range | | |
|---|---|---|---|
| Characteristic | | Min | Max |

| MMAD (by impaction) | 3.5 | μm | 7.5 | μm |
|---|---|---|---|---|
| Dispersibility (2/4 KPA | 0.5 | | 1.0 | |
| impaction delivered dose) | | | | |
| Delivered dose (% mass drug | 25.0% | | 80.0% | |
| product/active) | | | | |
| Bulk density | 0.2 | g/cm | 0.8 | g/cm |
| Tapped density | 0.3 | g/cm | 1.0 | g/cm³ |
| Moisture (by Karl Fischer) | 1.0% | w/w | 7.0% | w/w |

TABLE 4B
Exemplary Particle Characteristics

| | Range | | |
|---|---|---|---|
| Characteristic | | Min | Max |

| MMAD (by impaction) | 1.5 | μm | 7.5 | μm |
|---|---|---|---|---|
| Dispersibility (2/4 KPA | 0.5 | | 1.0 | |
| impaction delivered dose) | | | | |
| Delivered dose (% mass drug | 25.0% | | 125% | |
| product/active) | | | | |
| Bulk density | 0.1 | g/cm³ | 0.8 | g/cm³ |
| Tapped density | 0.1 | g/cm³ | 1.0 | g/cm³ |
| Moisture (by Karl Fischer) | 1.0% | w/w | 7.0% | w/w |

TABLE 5A
Exemplary Final Particle Characteristics

| Characteristic | Value | Units |
|---|---|---|
| MMAD (by impaction) | 4.2 | μm |
| Dispersibility (2/4 KPA impaction delivered dose) | 0.9 | — |
| Delivered dose (% mass drug product/active) | 60.0 | % |
| Bulk density | 0.5 | g/cm³ |
| Tapped density | 0.6 | g/cm³ |
| Moisture (by Karl Fischer) | 2.8 | % water |

TABLE 5B
Exemplary Final Particle Characteristics

| Characteristic | Value | Units |
|---|---|---|
| MMAD (by impaction) | 2.4 | μm |
| Dispersibility (2/4 KPA impaction delivered dose) | 0.9 | — |
| Delivered dose (% mass drug product/active) | 88 | % |
| Bulk density | 0.26 | g/cm³ |
| Tapped density | 0.30 | g/cm³ |
| Moisture (by Karl Fischer) | 1.48 | % water |

In some embodiments, the dried particles have a Dv50 of at least 0.5 μm. In some embodiments, the dried particles have a Dv50 of at least 2.0 μm. Dv50 is the size at which 50% of the particles are smaller than that size. Dv50 is effectively the median particle diameter, as measured by the technique used. In some embodiments, Dv50 is measured via laser diffraction techniques. Dv50 can apply to aerosolized particle or liquid suspended particles. Dv50 is purely volumetric in nature and does not take into account aerosol properties of the particle. Dv50 can be a useful measure to estimate dispersibility of the powder. Dispersibility refers to how readily a powder aerosolizes at different pressures. A perfectly dispersible powder can aerosolize consistently independent of the pressure applied. In some embodiments, the dried particles have a Dv50 of at least 0.5 um, at least 0.6 um, at least 0.7 um, at least 0.8 um, at least 0.9 um, 1 μm, at least 1.5 μm, at least 2 μm, at least 2.5 μm, at least 3 μm, at least 3.5 μm, at least 4 μm, at least 4.5 μm, at least 5 μm, at least 5.5 μm, at least 6 μm, at least 6.5 μm, at least 7 μm, at least 7.5 μm, at least 8 μm, at least 8.5 μm at least 9 μm, at least 9.5 μm, or at least 10 μm.

In some embodiments, the dried particles have a median mass aerodynamic diameter (MMAD) of at least 1.5 μm to at most 7.5 μm. In some embodiments, the dried particles have a median mass aerodynamic diameter (MMAD) of at least 3.5 μm to at most 7.5 μm. In some embodiments, the dried particles have a median mass aerodynamic diameter (MMAD) of at least 2.0 μm to at most 7.5 μm. In some embodiments, the dried particles have a median mass aerodynamic diameter (MMAD) of at least 2.5 μm to at most 7.5 μm. In some embodiments, the dried particles have a median mass aerodynamic diameter (MMAD) of at least 1.0 μm to at most 10 μm. As used herein, the term “MMAD” refers to the value of aerodynamic diameter for which 50% of some quantity in a given aerosol is associated with particles smaller than the MMAD, and 50% of the quantity is associated with particles larger than the MMAD. In some embodiments, the dried particles have a median mass aerodynamic diameter (MMAD) of at least 4.0 μm. In some embodiments, the dried particles have a median mass aerodynamic diameter (MMAD) of at least 4.2 μm. In some embodiments, the dried particles have a median mass aerodynamic diameter (MMAD) of at least 1 μm, at least 1.5 μm, at least 2 μm, at least 2.5 μm, at least 3 μm, at least 3.5 μm, at least 4 μm, at least 4.5 μm, at least 5 μm, at least 5.5 μm, at least 6 μm, at least 6.5 μm, at least 7 μm, at least 7.5 μm, at least 8 μm, at least 8.5 μm at least 9 μm, at least 9.5 μm, or at least 10 μm. In some embodiments, the dried particles have a median mass aerodynamic diameter (MMAD) of at most 1 μm, at most 1.5 μm, at most 2 μm, at most 2.5 μm, at most 3 μm, at most 3.5 μm, at most 4 μm, at most 4.5 μm, at most 5 μm, at most 5.5 μm, at most 6 μm, at most 6.5 μm, at most 7 μm, at most 7.5 μm, at most 8 μm, at most 8.5 μm at most 9 μm, at most 9.5 μm, or at most 10 μm.

In some embodiments, the dried particles described herein have a fine particle fraction (FPF)<5.0 μm of 94%. In some embodiments, the dried particles described herein have a fine particle fraction<5.0 μm of at least 70%, at least 75%, at least 80%, at least 85%, at least 90%, at least 91%, at least 92%, at least 93%, at least 94%, at least 95%, at least 96%, at least 97%, at least 98%, at least 99%, or more. In some embodiments, the dried particles described herein have a fine particle fraction<3.3 μm of 73%. In some embodiments, the dried particles described herein have a fine particle fraction<3.3 μm of at least 50%, at least 55%, at least 60%, at least 65%, at least 70%, at least 75%, at least 80%, at least 85%, at least 90%, at least 95%, or more.

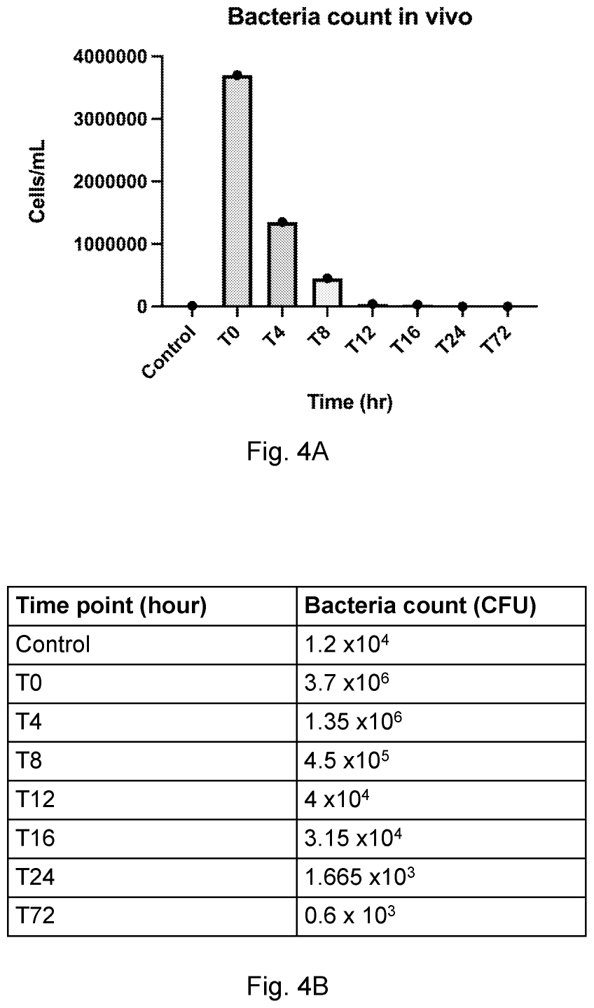

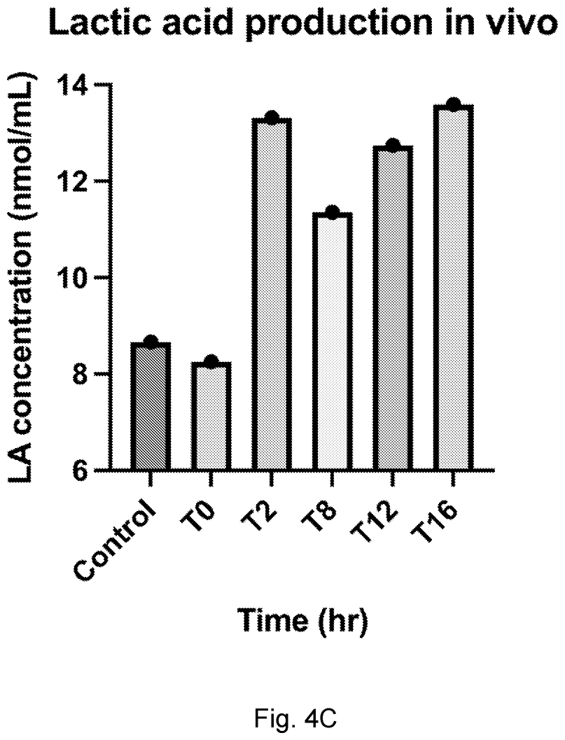

Fine particle fraction (FPF) is a value obtained from cascade impaction testing of a dry powder. In a cascade impaction test, powder is run across stages of different, progressively smaller sizes. The amount of powder deposited at each stage indicates the size cutoff percentages for powder as a whole. Two such methods are Next Generation Impaction (NGI) which flows powder horizontally across stages and Anderson Cascade Impaction (ACI) which flows powder vertically across stages. Typically, two values are provided: the % of the delivered dose smaller than 5.0 μm and the % of the delivered dose smaller than 3.3 um. <5.0 μm is the size cutoff typically regarded to deposit in the central and distal airways and <3.3 μm typically deposited in the distal airways. See e.g., Bianco et al., Respiratory Research volume 22, Article number: 71 (2021) (see e.g., FIG. 4 of Bianco); Darquenne, J Aerosol Med Pulm Drug Deliv. 2012; 25(3): 140-147; next generation impactor description (available on the world at sanyo-si.com/wp-content/uploads/NGI.pdf); the contents of each of which are incorporated herein by reference in their entireties.

In some embodiments, the dried particles have a dispersibility of less than 2.0. In some embodiments, the dried particles have a dispersibility of at least 0.5 to 1.0. As used herein, the term “dispersibility” refers to the ability of a particle to be spatially well-distributed, in terms of size and concentration, in a liquid after a controlled dispersion process. Dispersibility is unit-less. Dispersibility represents a ratio of the particle size measured for the same powder at different air pressures used to send the particles through the system. Specifically, dispersibility is a measurement taken by the Dv50 of a powder sent through a system at a “low” pressure, divided by the Dv50 of a powder sent through a system at a “high” pressure. In some embodiments, the dispersibility of the dried particles is measured as Dv50 measured at a pressure of 2 kPa divided by Dv50 measured at a pressure of 4 kPa (such a measurement can be referred to as “2/4 KPA herein”). Dispersibility can be measured for a delivered (e.g., emitted) dose. Dv50 can be measured either via impaction, laser diffraction (e.g., RODOS or MASTERSIZER), or both. Low pressure systems can have a higher Dv50 when measured using laser diffraction. Bulk and particle density affect dispersibility, as well as “stickiness” and shape of the dried particles, and vice versa. Because dispersibility comprises so many other attributes, dispersibility is a good dimensionless measure of how “well” a particle becomes airborne even if the pressure from air applied to it is not constant. In some embodiments, an “optimal” powder has a dispersibility ratio at 1 or close to 1, meaning its Dv50 does not change across air pressures in an aerodynamic system. As a ratio, the minimum value for dispersibility is zero; dispersibility can be less than 1 or greater than 1.

In some embodiments, the dried particles have a dispersibility of at least 0.9. In some embodiments, the dried particles have a dispersibility of at least 0.50, at least 0.55, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, at least 0.99, at least 1.0, at least 1.1, at least 1.2, at least 1.3, at least 1.4, at least 1.5, at least 1.6, at least 1.7, at least 1.8, at least 1.9, or 2.0. In some embodiments, the dried particles have a dispersibility of 0.50-0.55, 0.55-0.60, 0.60-0.65, 0.65-0.70, 0.70-0.75, 0.75-0.80, 0.80-0.85, 0.85-0.90, 0.90-0.91, 0.91-0.92, 0.92-0.93, 0.93-0.94, 0.94-0.95, 0.95-0.96, 0.96-0.97, 0.97-0.98, 0.98-0.99, 0.99-1.0, 1.0-1.1, 1.1-1.2, 1.2-1.3, 1.3-1.4, 1.4-1.5, 1.5-1.6, 1.6-1.7, 1.7-1.8, 1.8-1.9, 1.9-2.0, 0.5-0.75, 0.75-1.0, 0.75-1.25, 1.0-1.25, 1.25-1.5, or 0.5-1.5.

In some embodiments, the dried particles have a delivered dose of at least 25.0% to at most 125% of the composition by mass to a target tissue. In some embodiments, the dried particles have a delivered dose of at least 25.0% to at most 80.0% of the composition by mass to a target tissue. As used herein, the term “delivered dose” refers to the percentage of the composition or active ingredient thereof (e.g., bacterial preparation) that is expelled from a delivery device (e.g., inhaler) and/or delivered to a target tissue. In some embodiments, the delivered dose can be greater than 100% when the expected dose in the delivery device (e.g., 1.0 mg) is less than the actual dose in the delivery device (e.g., 1.25 mg; e.g., due to over-filling), and greater than the expected dose (e.g., >1.0 mg; e.g., >100%) but less than the actual dose (e.g., 1.25 mg; e.g., <125%) is delivered to the target tissue. The factors that influence delivered dose are ultimately related to dispersibility, powder density, hygroscopicity, and static charge (e.g., some of these factors affect dispersibility more than other factors). As a non-limiting example, higher density particles can aerosolize and fly better than lower density particles, which can fly unpredictably or be too large. As another non-limiting example, excipients that make the powder less “flowable” or sticky due to being too hygroscopic can make less powder leave the capsule when the inhaler (e.g., a DPI) is actuated. It is anticipated that not all powder from the capsule containing the spray-dried biotherapeutic matrix composition as described herein can make it to the target tissue (e.g., alveoli). A certain portion of the composition can: stay in the capsule, stay in the device, or hit the tongue or esophagus, and thus not be delivered. In some embodiments, the delivered dose refers to the amount of spray-dried biotherapeutic matrix composition as described herein that leaves the device and capsule.

Delivered dose can be measured as the mass % of powder that escapes a delivery system and lands in the target region(s). Emitted dose is a good measurement of what escapes the delivery device in an actual system. Content uniformity of that emitted dose can be measured to ensure that what is emitted from the dosage device is consistent. In some embodiments, the dried particles have a delivered dose of at least 60% of the bacterial preparation by mass to a target tissue. In some embodiments, the dried particles have a delivered dose of at least 25%, at least 30%, at least 35%, at least 40%, at least 45%, at least 50%, at least 55%, at least 60%, at least 65%, at least 70%, at least 75%, at least 80% of the bacterial preparation by mass that is expelled from a delivery device (e.g., inhaler) and/or delivered to a target tissue.

In some embodiments, the particles have a bulk density of at least 0.1 g/cm³ to 0.8 g/cm³. In some embodiments, the particles have a bulk density of at least 0.2 g/cm³ to 0.8 g/cm³. As used herein, “bulk density” is defined as the mass of the many particles of the material divided by the total volume they occupy; the total volume includes particle volume, inter-particle void volume, and internal pore volume. In some embodiments, the particles have a bulk density of at least 0.5 g/cm³. In some embodiments, the particles have a bulk density of at least 0.1 g/cm³, at least 0.2 g/cm³, at least 0.3 g/cm³, at least 0.4 g/cm³, at least 0.5 g/cm³, at least 0.6 g/cm³, at least 0.7 g/cm³, or at least 0.8 g/cm³.

In some embodiments, the particles have a tapped density of at least 0.1 g/cm³ to 1.0 g/cm³. In some embodiments, the particles have a tapped density of at least 0.2 g/cm³ to 1.0 g/cm³. In some embodiments, the particles have a tapped density of at least 0.3 g/cm³ to 1.0 g/cm³. In some embodiments, the particles have a tapped density of at least 0.1 g/cm³. In some embodiments, the particles have a tapped density of at least 0.3 g/cm³. As used herein, “tapped density” refers to the bulk density of the powder after a specified compaction process, e.g., involving vibration of a container containing the powder of dried particles. In some embodiments, the particles have a tapped density of at least 0.6 g/cm³. In some embodiments, the particles have a tapped density of at least 0.1 g/cm³, at least 0.2 g/cm³, at least 0.3 g/cm³, at least 0.4 g/cm³, at least 0.5 g/cm³, at least 0.6 g/cm³, at least 0.7 g/cm³, at least 0.8 g/cm³, at least 0.9 g/cm³, or at least 1.0 g/cm³.

In some embodiments, the particles have a moisture content of at least 1.0% to 7.0% water by weight. As used herein, “moisture content” refers to how much water is in a product, measured as a percentage. The moisture content can influence the physical properties of a substance, including weight, density, viscosity, conductivity, and others. Moisture content can be determined by weight loss upon drying (loss-on drying, LOD). In some embodiments, moisture content is measured using Karl Fischer Titration. Karl Fischer uses coulometric or volumetric titration to determine trace amounts of water in a sample. The elementary reaction responsible for water quantification in the Karl Fischer titration is oxidation of sulfur dioxide with iodine: H₂O+SO₂+I₂→SO₃+2HI. This elementary reaction consumes exactly one molar equivalent of water vs. iodine. Iodine is added to the solution until it is present in excess, marking the end point of the titration, which can be detected by potentiometry. The reaction is run in an alcohol solution containing a base, which consumes the sulfur trioxide and hydroiodic acid produced. The end point is detected most commonly by a bipotentiometric titration method.

In some embodiments, the particles have a moisture content of at least 1.0% water by weight (e.g., measured by Karl Fischer method). In some embodiments, the particles have a moisture content of at least 2.5% water by weight (e.g., measured by Karl Fischer method). In some embodiments, the particles have a moisture content of at most 2.5% water by weight. In some embodiments, the particles have a moisture content of at least 2.8% water by weight. In some embodiments, the particles have a moisture content of at most 2.8% water by weight. In some embodiments, the particles have a moisture content of at least 1%, at least 1.5%, at least 2%, at least 2.5%, at least 3%, at least 3.5%, at least 4%, at least 4.5%, at least 5%, at least 5.5%, at least 6%, at least 6.5%, at least 7% water by weight. In some embodiments, the particles have a moisture content of at most 1%, at most 1.5%, at most 2%, at most 2.5%, at most 3%, at most 3.5%, at most 4%, at most 4.5%, at most 5%, at most 5.5%, at most 6%, at most 6.5%, at most 7% water by weight.

### Devices

In one aspect, the spray-dried biotherapeutic matrix composition as described herein is in combination with a delivery device, such as an inhaler. In one aspect, described herein is an inhalation device for bronchopulmonary delivery comprising: (a) an inhaler; and (b) a container containing a spray-dried biotherapeutic matrix composition as described herein. In some embodiments, the inhaler comprises: (a) a mouthpiece comprising an opening; and (b) means for aerosolizing or dispersing the pharmaceutical composition in the container. In some embodiments, the means for aerosolizing or dispersing include, but are not limited to propellants, pressurized air, a spring, or another chemical or mechanical production of pressure. In some embodiments, the inhaler is a dry powder inhaler (DPI). In some embodiments, the inhaler is a metered dose inhaler (MDI). In some embodiments, the inhaler is a soft mist inhaler (SMI).

In some embodiments, the spray-dried biotherapeutic matrix composition as described herein is administered as an aerosolized composition. Aerosolization is the process or act of converting some physical substance (e.g., a solid) into the form of particles small and light enough to be carried on the air i.e. into an aerosol. In some embodiments, the spray-dried biotherapeutic matrix composition as described herein can also be administered directly to the airways in the form of a dry powder. For use as a dry powder, the spray-dried biotherapeutic matrix composition as described herein can be administered by use of an inhaler. Inhalers are portable, handheld devices that can be available as metered dose inhalers (MDI), dry powder inhalers (DPIs), or soft mist inhalers (SMI).

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The metered dose inhaler allows for the correct dosage of the composition to be delivered to the patient. While propellants and high pressures are not generally conducive to maintaining viability of bacteria, MDIs can be used to deliver non-viable bacteria (e.g., heat-killed bacteria), bacterial extracts, and/or bacterial products. In embodiments comprising viable bacteria, a DPI can be used instead of an MDI.

A dry powder inhaler (i.e. TURBUHALER (ASTRA AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of the spray-dried biotherapeutic matrix composition as described herein thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

A soft mist inhaler (SMI; e.g., Respimat® Soft Mist™ Inhaler) is a multidose, propellant-free, hand-held, liquid inhaler. The aerosol cloud generated by an SMI contains a higher fraction of fine particles than most pressurized metered dose inhalers (pMDIs) and dry powder inhalers (DPIs), and the aerosol spray exits the inhaler more slowly and for a longer duration than with pMDIs. This translates into higher lung drug deposition and lower oropharyngeal deposition, allowing lower nominal doses of delivered drugs without lowering efficacy. In some embodiments, the inhaler formulation does not comprise a propellant. Medication is stored as a solution in the SMI drug cartridge, e.g., an aluminum cylinder containing a double-walled, plastic, collapsible bag that contracts as the solution is used. The SMI solution can be formulated with ethanol or water, and preservatives (e.g., benzalkonium chloride or ethylene diamine tetra-acetic acid (EDTA)). The amount of preservatives in each administration (e.g., puff) can be about 0.44 μg for benzalkonium chloride or about 2.2 μg for EDTA.

In some embodiments of any of the aspects, the spray-dried biotherapeutic matrix composition as described herein is administered using a nasal spray or a nebulizer. In some embodiments of any of the aspects, the spray-dried biotherapeutic matrix composition is formulated as a nasal spray. In one aspect, the spray-dried biotherapeutic matrix composition as described herein is in combination with a nebulizer. In some embodiments of any of the aspects, the spray-dried biotherapeutic matrix composition is formulated for delivery by a nebulizer.

A nasal spray typically comprises a saline solution comprising the spray-dried biotherapeutic matrix composition as described herein, which is sprayed as a mist into the nasal passages using a mechanical spray nozzle. Nebulizers are electric- or battery-powered machines that convert a liquid spray-dried biotherapeutic matrix composition as described herein into a fine mist that is inhaled into the lungs. The inhaler or nebulizer described herein can further comprise a mouthpiece or facemask. A nasal spray typically administers the spray-dried biotherapeutic matrix composition as described herein to the upper respiratory tract, whereas the inhaler or nebulizer typically administers the spray-dried biotherapeutic matrix composition as described herein to the lower respiratory tract.

A spray-dried biotherapeutic matrix composition as described herein can be administered directly to the airways of a subject in the form of an aerosol or by nebulization. For use as aerosols, a spray-dried biotherapeutic matrix composition as described herein in solution or suspension can be packaged in a pressurized aerosol container together with suitable propellants, for example, hydrocarbon propellants like propane, butane, or isobutane with conventional adjuvants. The spray-dried biotherapeutic matrix composition as described herein can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multi-dose device. Nebulized solutions containing active pharmaceutical ingredients are well-suited for infant or adolescent lungs, as well as patients with weaker lung capacity. Nebulized solutions can face stability challenges and have size limitations that can prevent aerosolization during dosing.

As is well known, any suitable gas can be used to apply pressure during the nebulization, with preferred gases to date being those which are chemically inert to the spray-dried biotherapeutic matrix composition as described herein. Exemplary gases including, but are not limited to, nitrogen, argon or helium can be used to high advantage.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), the contents of each of which are incorporated herein by reference in their entirety.

### Spray-Drying Methods

Described herein are methods of preparing a spray-dried pharmaceutical compositions, e.g., which comprise a bacterial preparation as described herein. In one aspect, described herein is a method of preparing a spray-dried pharmaceutical composition comprising a bacterial preparation, comprising: (a) preparing a liquid feedstock comprising the bacterial preparation; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the bacterial preparation.

In one aspect, described herein is a method of preparing a spray-dried pharmaceutical composition comprising a bacterial preparation, comprising: (a) obtaining a liquid feedstock comprising the bacterial preparation; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the bacterial preparation. In some embodiments, the method of preparing a spray-dried pharmaceutical composition comprising a bacterial preparation is performed using at least one of the conditions described in Table 6 or Table 7A-7B.

TABLE 6
Exemplary Ranges of Spray Drying Process Conditions

| | Range | | |
|---|---|---|---|
| Condition | | Min | Max |

| | | | | |
|---|---|---|---|---|
| Liquid feedstock solids | 1 | g/L | 100 | g/L |
| concentration | | | | |
| Process gas inlet temperature | 100° | C. | 195° | C. |
| Process gas outlet temperature | 40° | C. | 85° | C. |
| Atomization gas pressure | 10 | psig | 150 | psig |
| Drying gas flow rate | 5 | kg/hr | 150 | kg/hr |
| Feedstock flow rate | 5 | g/min | 1,000 | g/min |

TABLE 7A
Exemplary Spray Drying Process Conditions

| Condition | Value | Units |
|---|---|---|
| Process gas inlet temperature | 100 | ° C. |
| Process gas outlet temperature | 48 | ° C. |
| Atomization gas pressure | 20 | psig |
| Drying gas flow rate | 18 | kg/hr |
| Feedstock flow rate | 15 | g/min |

TABLE 7B
Exemplary Spray Drying Process Conditions

| Condition | Value | Units |
|---|---|---|
| Process gas inlet temperature | 100 | ° C. |
| Process gas outlet temperature | 50 | ° C. |
| Atomization gas pressure | 345 | kPa |
| Drying gas flow rate | 600 | L/min |
| Feedstock flow rate | 2.3 | mL/min |

The processing conditions tabulated above in Table 6 and Table 7A-7B provide exemplary ranges across the spray unit operation size. For example, the higher values of gas pressure and flow rate represent conditions closer to a full-scale production unit (i.e., large-scale), and the values on the lower end represent conditions closer to a bench-top or lab scale processing unit (i.e., small-scale).

The drying gas flow rate can be expressed as either kg/hr or L/min. The value of kg/hr can be calculated from the L/min value using the following formula: Mass (kg/hr)=[[density of air (g/L)/volume (L/min)]*(1 kg/1000 g)*(60 min/1 hr)]; the density of air is about 1.225 g/L (e.g., it can vary slightly with temperature, elevation, etc.). For example, 600 L/min is equivalent to 44.1 kg/hr. The value of L/min can be calculated from the kg/hr value using the following formula: Volume (L/min)=[[mass (kg/hr)/density of air (g/L)]*(1000 g/l kg)*(1 hr/60 min)]. For example, 18 kg/hr is equivalent to 245 L/min.

The feedstock flow rate can be expressed as g/min or mL/min; such rates can be converted depending on the density (e.g., g/mL) of the liquid feedstock. The atomization pressure can be expressed as psig, kPa, or other known units for pressure (1 psig=6.89475728 kPa; 345 kPa=˜50 psig).

To spray dry sensitive biotherapeutic described herein, target processing conditions are optimized for three characteristics: (1) constituent viability/activity/assay; (2) powder flowability and stability; and (3) aerodynamic size and properties (see e.g., Table 4 and Table 5 for exemplary properties of the spray-dried particles).

In order to protect constituent viability, powder is spray dried at the lowest possible outlet temperature, which is the effective temperature that the dry powder will experience. During drying, a phenomenon known as evaporative cooling keeps the solid constituents relatively cool compared to their heated surroundings. As particle formation completes, the particles heat up to some temperature between room temperature and the temperature of the inlet drying gas. It is this temperature (measured at the outlet of the spray drying chamber) that should be minimized. Pulling in the other direction is the water content of the powder, which can also be minimized to improve long-term stability and physical characteristics. However, higher outlet temperatures for the same formulation typically result from a dryer particle. These two process conditions can be optimized to maximize particle viability and minimize moisture content. In addition, reducing atomization pressure is advantageous to constituent viability; higher pressures put stress on live organisms and sensitive therapeutics, so the atomization pressure used to make droplets should be kept as low as reasonably possible to protect the constituents.

Aerodynamic properties are dominated by the included excipients, particle density, and particle size. The excipients can be chosen to allow for dispersibility (e.g., by addition of an excipient, such as an amino acid, like leucine) and stability (e.g., by addition of a high glass transition temperature, strongly soluble sugar, polyol, or ionic salt; e.g., by the addition of a stabilizer as described herein). Inclusion of agents to increase dispersibility also enhances bulk powder properties like flowability, making the capsule filling process more efficient.

During spray drying, the particle formation is driven by two factors: (1) evaporation rate of the solvent, and (2) solubility of the solutes. A dimensionless number, called the Peclet number (Pe), provides an indication of which of these two phenomena dominate the droplet drying process inside the spray drying chamber, Formula VIII:

$$\frac{Droplet evaporation rate}{Diffusion of solutes} = Pe$$

### (VIII) Peclet Number Calculation

When Peclet numbers are high, particles tend to be very low density, large, hollow, and brittle. This is because evaporation of the droplet dominates the movement of solutes to the droplet center (in most systems), leaving a “shell” of solutes. Morphology tends to be spherical. Low Peclet numbers result in small, high-density particles. Solute mass transfer dominates the system as these solids move to the center of the slowly evaporating droplet. Morphology tends to be crumpled.

Often, high process temperatures result in a faster evaporation rate. Lower process temperatures tend to do the opposite, resulting in a dense, crumpled particle, which is the target morphology for our application.

Lower amounts of dissolved solids in the feedstock tend to result in faster diffusion of solutes, which also contributes to a small, dense, crumpled particle. Larger amounts of solids restrict the rate at which solute diffusion can occur but contribute to higher material yield and improved processing.

### Feedstock Preparation

In some embodiments, the method of preparing a spray-dried pharmaceutical composition comprises preparing a liquid feedstock, e g., comprising the bacterial preparation. In some embodiments, the step of preparing the liquid feedstock comprises dissolving a solid feedstock into a solution. In some embodiments, the step of preparing the liquid feedstock comprises dissolving a solid feedstock into an aqueous solution. One example of a solid feedstock for use in preparing an inhaled bacterial preparation formulation, with the optimal processing condition at lab-scale, can be found below in Table 8.

TABLE 8A
Exemplary Solid Feedstock

| Constituent | % w/w |
|---|---|
| Lacticaseibacillus rhamnosus (350 × 10⁹ CFU/g) | 3.5 |
| Leucine | 45.75 |
| Trehalose | 45.75 |
| Polysorbate 80 | 5.00 |

TABLE 8B
Exemplary Solid Feedstock

| Constituent | Target Mass (g) | % w/w |
|---|---|---|
| Lacticaseibacillus rhamnosus | 1.866 | 6.48 |
| (392 × 10⁹ CFU/g) | | |
| Lactobacillus acidophilus (392 × | 4.354 | 15.12 |
| 10⁹ CFU/g) | | |
| Lactobacillus plantarum (392 × | 0.980 | 3.40 |
| 10⁹ CFU/g) | | |
| Leucine | 9.840 | 34.17 |
| Trehalose | 9.840 | 34.17 |
| Polysorbate 80 | 0.480 | 1.67 |
| Sodium citrate | 1.440 | 5.00 |

In some embodiments, the solid feedstock comprises: at least 3.5% bacterial preparation by weight; at least 91.5% excipient by weight; and at least 5% stabilizer by weight. In some embodiments, the solid feedstock comprises at least 3.5% bacterial preparation by weight. In some embodiments, the solid feedstock comprises at least 91.5% excipient by weight. In some embodiments, the solid feedstock comprises at least 5% stabilizer by weight.

In some embodiments, the solid feedstock comprises: at least 3.5% bacterial preparation by weight; at least 45.75% of a first excipient by weight; at least 45.75% of a second excipient by weight; and at least 5% stabilizer by weight. In some embodiments, the solid feedstock comprises: at least 45.75% of a first excipient by weight, and at least 45.75% of a second excipient by weight.

In some embodiments, the solid feedstock comprises: at least 3.5% bacterial preparation by weight; at least 5% excipient by weight; and at least 0.25% stabilizer by weight. In some embodiments, the solid feedstock comprises at least 25% bacterial preparation by weight. In some embodiments, the solid feedstock comprises at least 50% excipient by weight. In some embodiments, the solid feedstock comprises at least 1% stabilizer by weight.

In some embodiments, the solid feedstock comprises: at least 3.5% bacterial preparation by weight; at least 5% of a first excipient by weight; at least 5% of a second excipient by weight; and at least 0.25% stabilizer by weight. In some embodiments, the solid feedstock comprises: at least 30% of a first excipient by weight, and at least 30% of a second excipient by weight. In some embodiments, the solid feedstock comprises: at least 30% of a first excipient by weight, at least 30% of a second excipient by weight, and at least 2.5% of a third excipient by weight. In some embodiments, the three excipients are leucine, trehalose, and sodium citrate.

In some embodiments, the solid feedstock comprises at least 1% to at most 50% bacterial preparation by weight. In some embodiments, the solid feedstock comprises at least 1% to at most 5% bacterial preparation by weight. In some embodiments, the solid feedstock comprises: at least 1%, at least 1.5%, at least 2%, at least 2.5%, at least 3%, at least 3.5%, at least 4%, at least 4.5%, or at least 5% bacterial preparation by weight.

In some embodiments, the solid feedstock comprises at least 45% to at most 95% excipient by weight. In some embodiments, the solid feedstock comprises at least 45%, at least 50%, at least 55%, at least 60%, at least 65%, at least 70%, at least 75%, at least 80%, at least 85%, at least 90%, or at least 95% excipient by weight.

In some embodiments, the solid feedstock comprises at least 5%-60% of a first excipient by weight, and at least 5%-60% of a second excipient by weight. In some embodiments, the solid feedstock comprises at least 30%-60% of a first excipient by weight, and at least 30%-60% of a second excipient by weight. In some embodiments, the solid feedstock comprises at least 35%, at least 40%, at least 45%, at least 50%, at least 55%, or at least 60% of a first excipient by weight, and at least 35%, at least 40%, at least 45%, at least 50%, at least 55%, or at least 60% of a second excipient by weight. In some embodiments, the solid feedstock comprises at least 35%, at least 40%, at least 45%, at least 50%, at least 55%, or at least 60% of a first excipient by weight, and at least 30% of a second excipient by weight. In some embodiments, the solid feedstock comprises at least 30% of a first excipient by weight, and at least 35%, at least 40%, at least 45%, at least 50%, at least 55%, or at least 60% of a second excipient by weight.

In some embodiments, the solid feedstock comprises at least 0.25% to at most 10% stabilizer by weight. In some embodiments, the solid feedstock comprises at least 0.5% to at most 10% stabilizer by weight. In some embodiments, the solid feedstock comprises at least 0.25%, at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 8%, at least 9%, or at least 10% stabilizer by weight.

Exemplary liquid feedstock formulae are provided in Tables 9A-9B below; in some embodiments, this exemplary liquid feedstock formula is 3% w/w solids, or 30 g/L of a 1000 g feedstock suspension (i.e., approximately 1 L, assuming approximately 1 g per 1 mL; as such “1 L of the liquid feedstock” can be used interchangeably with “1000 g of the liquid feedstock”). Such an exemplary liquid feedstock can be scaled up to a full-scale production unit, e.g., using the same or substantially the same ratios of components.

TABLE 9A
Exemplary Liquid Feedstock Formula

| Constituent | Target Mass (g) | % w/w |
|---|---|---|
| Lacticaseibacillus rhamnosus | 1.050 | 0.105 |
| (350 × 10⁹ CFU/g) | | |
| Leucine | 13.725 | 1.373 |
| Trehalose | 13.725 | 1.373 |
| Polysorbate 80 | 1.500 | 0.150 |
| Water | 970 | 97.000 |

TABLE 9B
Exemplary Liquid Feedstock Formula

| Constituent | Target Mass (g) | % w/w |
|---|---|---|
| Lacticaseibacillus rhamnosus | 1.866 | 6.48 |
| (392 × 10⁹ CFU/g) | | |
| Lactobacillus acidophilus | 4.354 | 15.12 |
| (392 × 10⁹ CFU/g) | | |
| Lactobacillus plantarum | 0.980 | 3.40 |
| (392 × 10⁹ CFU/g) | | |
| Leucine | 9.840 | 34.17 |
| Trehalose | 9.840 | 34.17 |
| Polysorbate 80 | 0.480 | 1.67 |
| Sodium citrate | 1.440 | 5.00 |
| Water | 960 mL | — |

In some embodiments, the liquid feedstock comprises at least 1.0 g/L solid feedstock dissolved in an aqueous solution. In some embodiments, the liquid feedstock comprises at least 1.0 g/L to at most 100 g/L solid feedstock dissolved in an aqueous solution. In some embodiments, the liquid feedstock comprises at least 25 g/L solid feedstock dissolved in an aqueous solution. In some embodiments, the liquid feedstock comprises at least 30 g/L solid feedstock dissolved in an aqueous solution. In some embodiments, the liquid feedstock comprises at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 20 g/L, at least 30 g/L, at least 40 g/L, at least 50 g/L, at least 60 g/L, at least 70 g/L, at least 80 g/L, at least 90 g/L, or at least 100 g/L solid feedstock dissolved in an aqueous solution.

In some embodiments, the liquid feedstock comprises at least 0.1% to at most 10% solid feedstock dissolved in an aqueous solution. In some embodiments, the liquid feedstock comprises at least 1% solid feedstock dissolved in an aqueous solution. In some embodiments, the liquid feedstock comprises at least 3% solid feedstock dissolved in an aqueous solution. In some embodiments, the liquid feedstock comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% or more solid feedstock dissolved in an aqueous solution.

In some embodiments, 1 L of the liquid feedstock comprises: (a) at least 1.050 g bacterial preparation; (b) at least 1.5 g excipient; (c) at least 0.075 g stabilizer; and/or (d) at least 970 g aqueous solution. In some embodiments, 1 L of the liquid feedstock comprises: (a) at least 1.050 g bacterial preparation; (b) at least 0.75 g of a first excipient; (c) at least 0.75 g of a second excipient; (d) at least 0.075 g stabilizer; and/or (e) at least 970 g aqueous solution.

In some embodiments, 1 L of the liquid feedstock comprises: (a) at least 1.050 g bacterial preparation; (b) at least 0.5 g of a first excipient; (c) at least 0.5 g of a second excipient; (d) at least 0.5 g of a third excipient; (e) at least 0.075 g stabilizer; and/or (f) at least 970 g aqueous solution. In some embodiments, 1 L of the liquid feedstock comprises: (a) at least 1.050 g bacterial preparation; (b) at least 0.75 g of a first excipient; (c) at least 0.75 g of a second excipient; (d) at least 0.75 g of a third excipient; (e) at least 0.075 g stabilizer; and/or (f) at least 970 g aqueous solution.

In some embodiments, 1 L of the liquid feedstock comprises: (a) at least 1.050 g bacterial preparation; (b) at least 27.45 g excipient; (c) at least 1.5 g stabilizer; and/or (d) at least 970 g aqueous solution. In some embodiments, 1 L of the liquid feedstock comprises: (a) at least 1.050 g bacterial preparation; (b) at least 13.725 g of a first excipient; (c) at least 13.725 g of a second excipient; (d) at least 1.5 g stabilizer; and/or (e) at least 970 g aqueous solution.

In some embodiments, 1 L of the liquid feedstock comprises at least 0.1 g to at most 15 g bacterial preparation (e.g., to account for a 50% API loading). In some embodiments, 1 L of the liquid feedstock comprises at least 0.1 g to at most 10 g bacterial preparation. In some embodiments, 1 L of the liquid feedstock comprises at least 1 g bacterial preparation. In some embodiments, 1 L of the liquid feedstock comprises at least 1.05 g bacterial preparation. In some embodiments, 1 L of the liquid feedstock comprises at least 2 g bacterial preparation. In some embodiments, 1 L of the liquid feedstock comprises at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, at least 5 g, at least 6 g, at least 7 g, at least 8 g, at least 9 g, or at least 10 g bacterial preparation.

In some embodiments, 1 L of the liquid feedstock comprises at least 10 g to at most 50 g excipient. In some embodiments, 1 L of the liquid feedstock comprises at least 0.75 g excipient. In some embodiments, 1 L of the liquid feedstock comprises at least 25 g excipient. In some embodiments, 1 L of the liquid feedstock comprises at least 20 g excipient. In some embodiments, 1 L of the liquid feedstock comprises at least 27.45 g excipient. In some embodiments, 1 L of the liquid feedstock comprises at least 10 g, at least 15 g, at least 20 g, at least 25 g, at least 30 g, at least 35 g, at least 40 g, at least 45 g, or at least 50 g excipient.

In some embodiments, 1 L of the liquid feedstock comprises at least 0.75 g to at most 25 g of a first excipient, and at least 0.75 g to at most 25 g of a second excipient. In some embodiments, 1 L of the liquid feedstock comprises at least 0.75 g to at most 25 g of a first excipient, at least 0.75 g to at most 25 g of a second excipient, and at least 0.75 g to at most 25 g of a third excipient. In some embodiments, 1 L of the liquid feedstock comprises at least 5 g to at most 25 g of a first excipient, and at least 5 g to at most 25 g of a second excipient. In some embodiments, 1 L of the liquid feedstock comprises at least 5 g to at most 25 g of a first excipient, at least 5 g to at most 25 g of a second excipient, and at least 5 g to at most 25 g of a third excipient. In some embodiments, 1 L of the liquid feedstock comprises at least 9.840 g of a first excipient and at least 1.440 g of a second excipient. In some embodiments, 1 L of the liquid feedstock comprises at least 13.725 g of a first excipient and at least 13.725 g of a second excipient. In some embodiments, 1 L of the liquid feedstock comprises at least 5 g, at least 10 g, at least 15 g, at least 20 g, or at least 25 g of a first excipient and at least 5 g, at least 10 g, at least 15 g, at least 20 g, or at least 25 g of a second excipient. In some embodiments, 1 L of the liquid feedstock comprises at least 5 g, at least 10 g, at least 15 g, at least 20 g, or at least 25 g of a first excipient and at least 5 g of a second excipient. In some embodiments, 1 L of the liquid feedstock comprises at least 5 g of a first excipient and at least 5 g, at least 10 g, at least 15 g, at least 20 g, or at least 25 g a second excipient.

In some embodiments, 1 L of the liquid feedstock comprises at least 0.075 g to at most 10 g stabilizer. In some embodiments, 1 L of the liquid feedstock comprises at least 0.1 g to at most 10 g stabilizer. In some embodiments, 1 L of the liquid feedstock comprises at least 0.075 g stabilizer. In some embodiments, 1 L of the liquid feedstock comprises at least 1.5 g stabilizer. In some embodiments, 1 L of the liquid feedstock comprises at least 0.075 g, at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, at least 5 g, at least 6 g, at least 7 g, at least 8 g, at least 9 g, or at least 10 g stabilizer.

In some embodiments, 1 L of the liquid feedstock comprises at least 750 g to at most 999 g aqueous solution. In some embodiments, 1 L of the liquid feedstock comprises at least 900 g to at most 999 g aqueous solution. In some embodiments, 1 L of the liquid feedstock comprises at least 750 g aqueous solution. In some embodiments, 1 L of the liquid feedstock comprises at least 970 g aqueous solution. In some embodiments, 1 L of the liquid feedstock comprises at least 900 g, at least 905 g, at least 910 g, at least 915 g, at least 920 g, at least 925 g, at least 930 g, at least 935 g, at least 940 g, at least 945 g, at least 950 g, at least 955 g, at least 960 g, at least 965 g, at least 970 g, at least 975 g, at least 980 g, at least 985 g, at least 990 g, at least 995 g, or at least 999 g aqueous solution.

In some embodiments, the liquid feedstock comprises: (a) at least 0.105% bacterial preparation; (b) at least 0.150% excipient; (c) at least 0.0075% stabilizer; and/or (d) at least 97% aqueous solution. In some embodiments, the liquid feedstock comprises: (a) at least 0.105% bacterial preparation by weight; (b) at least 0.075% of a first excipient by weight; (c) at least 0.075% of a second excipient by weight; (d) at least 0.0075% stabilizer and/or (e) at least 97% aqueous solution by weight.

In some embodiments, the liquid feedstock comprises: (a) at least 0.105% bacterial preparation by weight; (b) at least 0.05% of a first excipient by weight; (c) at least 0.05% of a second excipient by weight; (d) at least 0.05% of a third excipient by weight; (e) at least 0.0075% stabilizer and/or (f) at least 97% aqueous solution by weight. In some embodiments, the liquid feedstock comprises: (a) at least 0.105% bacterial preparation by weight; (b) at least 0.075% of a first excipient by weight; (c) at least 0.075% of a second excipient by weight; (d) at least 0.075% of a third excipient by weight; (e) at least 0.0075% stabilizer and/or (f) at least 97% aqueous solution by weight.

In some embodiments, the liquid feedstock comprises: (a) at least 0.105% bacterial preparation; (b) at least 2.745% excipient; (c) at least 0.15% stabilizer; and/or (d) at least 97% aqueous solution. In some embodiments, the liquid feedstock comprises: (a) at least 0.105% bacterial preparation by weight; (b) at least 1.3725% of a first excipient by weight; (c) at least 1.3725% of a second excipient by weight; (d) at least 0.15% stabilizer and/or (e) at least 97% aqueous solution by weight.

In some embodiments, the liquid feedstock comprises at least 0.01% to at most 10% bacterial preparation by weight. In some embodiments, the liquid feedstock comprises at least 0.01% to at most 1.0% bacterial preparation by weight. In some embodiments, the liquid feedstock comprises at least 0.105% bacterial preparation by weight. In some embodiments, the liquid feedstock comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at most 10% bacterial preparation by weight.

In some embodiments, the liquid feedstock comprises at least 1% to at most 5% excipient by weight. In some embodiments, the liquid feedstock comprises at least 0.15% excipient by weight. In some embodiments, the liquid feedstock comprises at least 1%, at least 1.5%, at least 2%, at least 2.5%, at least 3%, at least 3.5%, at least 4%, at least 4.5%, or at least 5% excipient by weight.

In some embodiments, the liquid feedstock comprises at least 0.075% to at most 2.5% of a first excipient by weight, and at least 0.075% to at most 2.5% of a second excipient by weight. In some embodiments, the liquid feedstock comprises at least 0.075% of a first excipient by weight, and at least 0.075% of a second excipient by weight.

In some embodiments, the liquid feedstock comprises at least 1% to at most 20% excipient by weight. In some embodiments, the liquid feedstock comprises at least 1% to at most 5% excipient by weight. In some embodiments, the liquid feedstock comprises at least 2.745% excipient by weight. In some embodiments, the liquid feedstock comprises at least 1%, at least 1.5%, at least 2%, at least 2.5%, at least 3%, at least 3.5%, at least 4%, at least 4.5%, at least 5% excipient, at least 10%, at least 15%, or at least 20% by weight.

In some embodiments, the liquid feedstock comprises at least 0.1% to at most 19.8% of a first excipient by weight, and at least 0.10% to at most 19.8% of a second excipient by weight. In some embodiments, the liquid feedstock comprises at least 0.50% to at most 2.5% of a first excipient by weight, and at least 0.5% to at most 2.5% of a second excipient by weight. In some embodiments, the liquid feedstock comprises at least 1.3725% of a first excipient by weight, and at least 1.3725% of a second excipient by weight.

In some embodiments, the liquid feedstock comprises at least 0.075%, at least 0.5%, at least 1%, at least 1.5%, at least 2%, or at least 2.5%, or more of a first excipient by weight, and at least 0.075%, at least 0.5%, at least 1%, at least 1.5%, at least 2%, or at least 2.5%, or more of a second excipient by weight. In some embodiments, the liquid feedstock comprises at least 0.5%, at least 1%, at least 1.5%, at least 2%, or at least 2.5% or more of a first excipient by weight, and at least 0.5% of a second excipient by weight. In some embodiments, the liquid feedstock comprises at least 0.5% of a first excipient by weight, and at least 0.5%, at least 1%, at least 1.5%, at least 2%, or at least 2.5% or more of a second excipient by weight.

In some embodiments, the liquid feedstock comprises at least 0.0075% to at most 1.0% stabilizer by weight. In some embodiments, the liquid feedstock comprises at least 0.01% to at most 1.0% stabilizer by weight. In some embodiments, the liquid feedstock comprises at least 0.15% stabilizer by weight. In some embodiments, the liquid feedstock comprises at least 0.0075% stabilizer by weight. In some embodiments, the liquid feedstock comprises at least 0.05% stabilizer by weight. In some embodiments, the liquid feedstock comprises at least 0.045% stabilizer by weight. In some embodiments, the liquid feedstock comprises at least 0.0075%, at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% or more stabilizer by weight.

In some embodiments, the liquid feedstock comprises at least 75% to at most 99.9% aqueous solution by weight. In some embodiments, the liquid feedstock comprises at least 90% to at most 99.9% aqueous solution by weight. In some embodiments, the liquid feedstock comprises at least 97% aqueous solution by weight. In some embodiments, the liquid feedstock comprises at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or more aqueous solution by weight.

In some embodiments, the bacterial preparation comprises viable or non-viable bacteria. In some embodiments, the bacteria belong to a genus selected from the group consisting of: Carnobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella. In some embodiments, the bacterium is Lacticaseibacillus rhamnosus, Lactobacillus acidophilus, or Lactiplantibacillus plantarum. In some embodiments, the bacterium is Lacticaseibacillus rhamnosus. In some embodiments, the bacterium is Lactobacillus acidophilus. In some embodiments, the bacterium is Lactiplantibacillus plantarum. In some embodiments, the bacterium is Lacticaseibacillus rhamnosus and Lactobacillus acidophilus. In some embodiments, the bacterium is Lacticaseibacillus rhamnosus and Lactiplantibacillus plantarum. In some embodiments, the bacterium is Lactobacillus acidophilus and Lactiplantibacillus plantarum. In some embodiments, the bacterium is Lacticaseibacillus rhamnosus, Lactobacillus acidophilus, and Lactiplantibacillus plantarum. In some embodiments, the bacterium is Lacticaseibacillus rhamnosus strain LGG. In some embodiments, the bacterium is Lactiplantibacillus plantarum ATCC BAA-793™. In some embodiments, the bacterium is Lactobacillus acidophilus ATCC 4356™. In some embodiments, the bacterium is Lacticaseibacillus rhamnosus ATCC 53103™. In some embodiments, the bacteria are lactic acid bacteria (LAB), i.e., belong to the order Lactobacillales and produce lactic acid as the major metabolic end product of carbohydrate fermentation.

In some embodiments, the bacteria are non-pathogenic. In some embodiments, the bacteria are present at a concentration of at least 10¹ colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g. In some embodiments, the bacteria are present at a concentration of at least 10⁶ colony-forming units per gram (CFU/g). In some embodiments, the bacteria are present at a concentration of at least 350×10⁶ colony-forming units per gram (CFU/g).

In some embodiments, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts. In some embodiments, the excipient is leucine and/or trehalose.

In some embodiments, the stabilizer is a polysorbate; poloxamer; or polyvinyl alcohol. In some embodiments, the stabilizer is Polysorbate 80.

In some embodiments, the aqueous solution is water. In some embodiments, the aqueous solution comprises water.

In some embodiments, the liquid feedstock further comprises at least one additional therapeutic. Non-limiting examples of the at least one additional therapeutic include: an anti-inflammatory, an antimicrobial, an antiviral, an antifungal, a vasodilator, or a bronchodilator, as described herein. In some embodiments, the bacterial preparation and at least one additional therapeutic are spray-dried together. In some embodiments, the bacterial preparation and at least one additional therapeutic are spray-dried separately.

### Atomization

Described herein are methods of spray-drying a composition comprising a bacterial preparation. Such spray-drying can be performed using spray-drying equipment or technology known in the art. Methods of spray-drying are known in the art and are not limited; see e.g., U.S. Pat. Nos. 7,258,873, 7,378,110, 8,273,374, 8,293,275, 9,238,005, 9,044,497; US Patent Publications US20130022728, US20140086965, US20180027855; the contents of each of which are incorporated herein by reference in their entirety

In one aspect, the method of preparing a spray-dried pharmaceutical composition comprises: (a) introducing droplets of a liquid feedstock as describe herein through an atomization nozzle into a drying chamber; (b) exposing the liquid feedstock droplets to heated, pressurized gas in the drying chamber to create dried particles; and (c) isolating dried particles of a predetermined range of diameters in a cyclone chamber, wherein the isolated dried particles comprise the bacterial preparation.

In some embodiments, the method of preparing a spray-dried pharmaceutical composition comprises introducing droplets of a liquid feedstock as describe herein through an atomization nozzle into a drying chamber. In some embodiments, the atomization nozzle into the drying chamber has a diameter of at least 1.2 mm. Some commercial, full-scale nozzles can have much larger nozzle diameters than 1.2 mm, but spray at much higher atomization pressures (e.g., greater than 150 psig) to re-produce the ideal droplet sizes and dried particle characterizations described herein. In some embodiments, the atomization nozzle into the drying chamber has a diameter of at least 0.5 mm, at least 1 mm, at least 1.5 mm, at least 2 mm, at least 2.5 mm, at least 3 mm, at least 3.5 mm, at least 4 mm, at least 4.5 mm, or at least 5 mm.

In some embodiments, the droplets of liquid feedstock produced by the atomization nozzle into the drying chamber have a diameter of at least 1.5 um. In some embodiments, the droplets of liquid feedstock produced by the atomization nozzle into the drying chamber have a diameter of at least 3.5 um. In some embodiments, the droplets of liquid feedstock produced by the atomization nozzle into the drying chamber have a diameter of at least 0.5 um, at least 1 um, at least 1.5 um, at least 2 um, at least 2.5 um, at least 3 um, at least 3.5 um, at least 4 um, at least 4.5 um, or at least 5 um.

### Drying Chamber

In some embodiments, the method of preparing a spray-dried pharmaceutical composition comprises exposing the liquid feedstock droplets to heated, pressurized gas in a drying chamber to create dried particles.

In some embodiments, the droplets of liquid feedstock have a flow rate through the drying chamber of at least 0.5 g/min. In some embodiments, the droplets of liquid feedstock have a flow rate through the drying chamber of at least 5 g/min. In some embodiments, the droplets of liquid feedstock have a flow rate through the drying chamber of at least 15 g/min. In some embodiments, the droplets of liquid feedstock have a flow rate through the drying chamber of at most 1000 g/min. In some embodiments, the droplets of liquid feedstock have a flow rate through the drying chamber of at least 0.5 g/min, at least 1 g/min, at least 2 g/min, at least 3 g/min, at least 4 g/min, at least 5 g/min, at least 10 g/min, at least 15 g/min, at least 20 g/min, at least 25 g/min, at least 30 g/min, at least 35 g/min, at least 40 g/min, at least 45 g/min, at least 50 g/min, at least 60 g/min, at least 70 g/min, at least 80 g/min, at least 90 g/min, at least 100 g/min, at least 200 g/min, at least 300 g/min, at least 400 g/min, at least 500 g/min, at least 600 g/min, at least 700 g/min, at least 800 g/min, at least 900 g/min, or at least 1000 g/min.

In some embodiments, the heated, pressurized gas is filtered before being inlet into the drying chamber. In some embodiments, the filter is a high efficiency particulate air (HEPA) filter. In some embodiments, the filter removes contaminants of at least 0.3 um. In some embodiments, the filter removes contaminants of at least 0.01 um, at least 0.02 um, at least 0.03 um, at least 0.04 um, at least 0.05 um, at least 0.06 um, at least 0.07 um, at least 0.08 um, at least 0.09 um, at least 0.1 um, at least 0.2 um, at least 0.3 um, at least 0.4 um, at least 0.5 um, at least 0.6 um, at least 0.7 um, at least 0.8 um, at least 0.9 um, or at least 1 um.

In some embodiments, the heated, pressurized gas is heated before being inlet into the drying chamber. In some embodiments, the heated, pressurized gas is inlet into the drying chamber at a temperature of at least 100° C. In some embodiments, the heated, pressurized gas is inlet into the drying chamber at a temperature of at least 135° C. In some embodiments, the heated, pressurized gas is inlet into the drying chamber at a temperature of at most 195° C. In some embodiments, the heated, pressurized gas is inlet into the drying chamber at a temperature of at least 100° C., at least 105° C., at least 110° C., at least 115° C., at least 120° C., at least 125° C., at least 130° C., at least 135° C., at least 140° C., at least 145° C., at least 150° C., at least 155° C., at least 160° C., at least 165° C., at least 170° C., at least 175° C., at least 180° C., at least 185° C., at least 190° C., or at least 195° C.

In some embodiments, the heated, pressurized gas is outlet from the drying chamber at a temperature of at least 40° C. In some embodiments, the heated, pressurized gas is outlet from the drying chamber at a temperature of at least 48° C. In some embodiments, the heated, pressurized gas is outlet from the drying chamber at a temperature of at least 50° C. In some embodiments, the heated, pressurized gas is outlet from the drying chamber at a temperature of at most 50° C. In some embodiments, the heated, pressurized gas is outlet from the drying chamber at a temperature of at least 60° C. In some embodiments, the heated, pressurized gas is outlet from the drying chamber at a temperature of at most 60° C. In some embodiments, the heated, pressurized gas is outlet from the drying chamber at a temperature of at most 85° C. In some embodiments, the heated, pressurized gas is outlet from the drying chamber at a temperature of at least 40° C., at least 45° C., at least 50° C., at least 55° C., at least 60° C., at least 65° C., at least 70° C., at least 75° C., at least 80° C., or at least 85° C.

In some embodiments, the heated, pressurized gas is pressurized before being inlet into the drying chamber. In some embodiments, the heated, pressurized gas in the drying chamber has an atomization gas pressure of at least 10 pounds per square inch gauge (psig). In some embodiments, the heated, pressurized gas in the drying chamber has an atomization gas pressure of at least 20 pounds per square inch gauge (psig). In some embodiments, the heated, pressurized gas in the drying chamber has an atomization gas pressure of at most 150 pounds per square inch gauge (psig). In some embodiments, the heated, pressurized gas in the drying chamber has an atomization gas pressure of at least 10 psig, at least 20 psig, at least 30 psig, at least 40 psig, at least 50 psig, at least 60 psig, at least 70 psig, at least 80 psig, at least 90 psig, at least 100 psig, at least 110 psig, at least 120 psig, at least 130 psig, at least 140 psig, at least 150 psig.

In some embodiments, the heated, pressurized gas has a flow rate through the drying chamber of at least 5 kg/hr. In some embodiments, the heated, pressurized gas has a flow rate through the drying chamber of at least 18 kg/hr. In some embodiments, the heated, pressurized gas has a flow rate through the drying chamber of at least 44 kg/hr. In some embodiments, the heated, pressurized gas has a flow rate through the drying chamber of at most 150 kg/hr. In some embodiments, the heated, pressurized gas has a flow rate through the drying chamber of at least 5 kg/hr, at least 6 kg/hr, at least 7 kg/hr, at least 8 kg/hr, at least 9 kg/hr, at least 10 kg/hr, at least 11 kg/hr, at least 12 kg/hr, at least 13 kg/hr, at least 14 kg/hr, at least 15 kg/hr, at least 16 kg/hr, at least 17 kg/hr, at least 18 kg/hr, at least 19 kg/hr, at least 20 kg/hr, at least 30 kg/hr, at least 40 kg/hr, at least 50 kg/hr, at least 60 kg/hr, at least 70 kg/hr, at least 80 kg/hr, at least 90 kg/hr, at least 100 kg/hr, at least 110 kg/hr, at least 120 kg/hr, at least 130 kg/hr, at least 140 kg/hr, at least 150 kg/hr.

Spray drying can be performed in batches, depending on the stability of the feedstock and availability to run the dryer for a certain amount of time. In some embodiments, spray-drying batches last an 8-hour day, but can be longer depending on the manufacturer. After this time, the equipment is cleaned and prepared for another run with “fresh” equipment. This batch time depends on the size of the drying chamber, which determines the scale at which one can spray dry (assuming the drying gas flow rate and temperature can be increased to match). In some embodiments, the step of exposing the liquid feedstock droplets to heated, pressurized gas in the drying chamber takes at most 8 hours, e.g., 8 hours per batch cycle.

In some embodiments, the step of exposing the liquid feedstock droplets to heated, pressurized gas in the drying chamber takes at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, or at least 24 hours. In some embodiments, the step of exposing the liquid feedstock droplets to heated, pressurized gas in the drying chamber takes at most 1 hour, at most 2 hours, at most 3 hours, at most 4 hours, at most 5 hours, at most 6 hours, at most 7 hours, at most 8 hours, at most 9 hours, at most 10 hours, at most 11 hours, at most 12 hours, at most 18 hours, or at most 24 hours.

In order to establish a gram-per-minute powder production rate, this can be converted from the solids flowrate into the drying chamber via two values: 1) the mass concentration in the feedstock, and 2) the mass flowrate of feedstock into the drying chamber.

### Cyclone Chamber

In some embodiments, the method of preparing a spray-dried pharmaceutical composition comprises isolating dried particles of a predetermined range of diameters in a cyclone chamber. In some embodiments, the heated, pressurized gas is outlet through the cyclone chamber.

In some embodiments, the step of isolating dried particles of a predetermined range of diameters in a cyclone chamber occurs continuously. Powders that settle in the cyclone chamber and collection vessel at the bottom of the cyclone chamber do so continuously. Isolating does not necessarily take a set amount of time

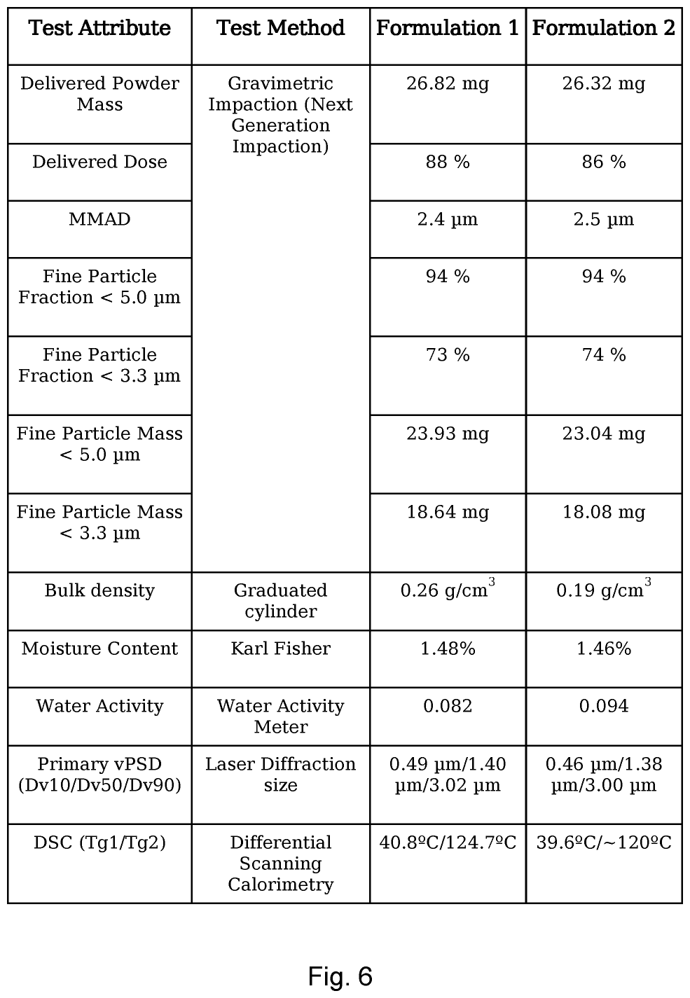

In some embodiments, the dried particles isolated in the cyclone chamber have a median mass aerodynamic diameter (MMAD) of at least 1.5 μm to at most 7.5 μm. In some embodiments, the dried particles isolated in the cyclone chamber have a median mass aerodynamic diameter (MMAD) of at least 2.5 μm to at most 7.5 μm. In some embodiments, the dried particles isolated in the cyclone chamber have a median mass aerodynamic diameter (MMAD) of at least 1.0 μm to at most 10.0 μm. In some embodiments, the dried particles isolated in the cyclone chamber have a median mass aerodynamic diameter (MMAD) of at least 2.3 μm. In some embodiments, the dried particles isolated in the cyclone chamber have a median mass aerodynamic diameter (MMAD) of about 2.4 μm to about 2.5 μm (see e.g., FIG. 6). In some embodiments, the dried particles isolated in the cyclone chamber have a median mass aerodynamic diameter (MMAD) of at least 4.0 μm. In some embodiments, the dried particles isolated in the cyclone chamber have a median mass aerodynamic diameter (MMAD) of at least 4.2 μm. In some embodiments, the dried particles isolated in the cyclone chamber have a median mass aerodynamic diameter (MMAD) of at least 5.0 μm. In some embodiments, the dried particles isolated in the cyclone chamber have a median mass aerodynamic diameter (MMAD) of at least 1 μm, at least 1.5 μm, at least 2 μm, at least 2.5 μm, at least 3 μm, at least 3.5 μm, at last 4.0 μm, at least 4.5 μm, at least 5.0 μm, at least 5.5 μm, at least 6.0 μm, at least 6.5 μm, at least 7.0 μm, at least 7.5 μm, at least 8.5 μm at least 9 μm, at least 9.5 μm, or at least 10 μm. In some embodiments, the dried particles isolated in the cyclone chamber have a median mass aerodynamic diameter (MMAD) of at most 1 μm, at most 1.5 μm, at most 2 μm, at most 2.5 μm, at most 3 μm, at most 3.5 μm, at last 4.0 μm, at most 4.5 μm, at most 5.0 μm, at most 5.5 μm, at most 6.0 μm, at most 6.5 μm, at most 7.0 μm, at most 7.5 μm, at most 8.5 μm at most 9 μm, at most 9.5 μm, or at most 10 μm.

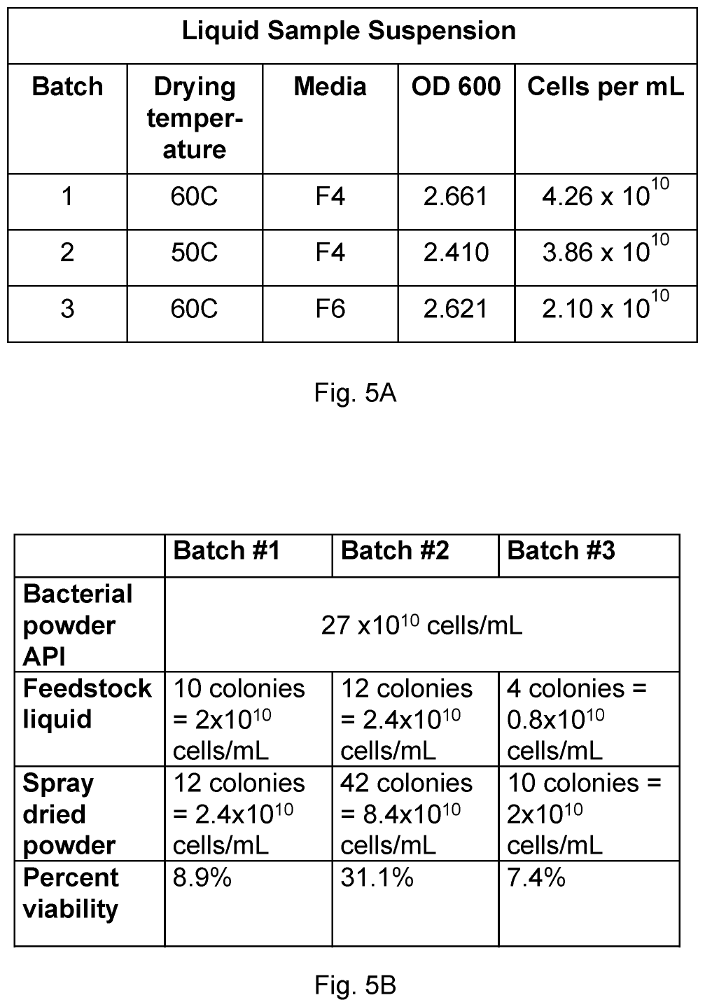

In some embodiments comprising viable bacterial preparations, the spray-dried biotherapeutic matrix composition comprises a bacterial viability of at least 5% after preparation using a spray drying method as described herein (see e.g., FIG. 5B). In some embodiments, the viability is determined after the step of isolating the dried particles. In some embodiments, the viability in the spray-dried biotherapeutic matrix composition (or dried particles thereof) is compared to the number of viable bacteria in the liquid feedstock prior to spray-drying. In embodiments, the spray-dried biotherapeutic matrix composition comprises a bacterial viability of at least 7.4% after preparation using a spray drying method as described herein. In embodiments, the spray-dried biotherapeutic matrix composition comprises a bacterial viability of at least 8.9% after preparation using a spray drying method as described herein. In embodiments, the spray-dried biotherapeutic matrix composition comprises a bacterial viability of at least 5% after preparation using a spray drying method as described herein. In embodiments, the spray-dried biotherapeutic matrix composition comprises a bacterial viability of at least 8.9% after preparation using a spray drying method as described herein.

In some embodiments, the spray-dried biotherapeutic matrix composition comprises a bacterial viability of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more after preparation using a spray drying method as described herein. In some embodiments, the spray-dried biotherapeutic matrix composition comprises a bacterial viability of 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, or 95%-100% after preparation using a spray drying method as described herein.

In embodiments such as those involving administration using a DPI, the spray-dried particles can be capsulated (i.e., made into capsules) following their isolation from the cyclone chamber of the spray drier.

### Administration

In some embodiments, the methods described herein relate to treating a subject having or diagnosed as having a bronchopulmonary disease. In some embodiments, the subject has or is diagnosed as having a chronic bronchopulmonary disease. In some embodiments, the subject has or is diagnosed as having an infectious bronchopulmonary disease. Subjects having a chronic bronchopulmonary disease can be identified by a physician using current methods of diagnosing a chronic bronchopulmonary disease. Symptoms and/or complications of a chronic bronchopulmonary disease which characterize these conditions and aid in diagnosis are well known in the art and include but are not limited to, difficulty breathing; shortness of breath, especially during physical activities; wheezing; chest tightness; a chronic cough that can produce mucus (sputum); frequent respiratory infections; lack of energy; unintended weight loss; and/or swelling in ankles, feet or legs. Tests that may aid in a diagnosis of, e.g. a chronic bronchopulmonary disease include, but are not limited to, lung (pulmonary) function tests; chest x-rays; a lung CT scan; arterial blood gas analysis; and/or laboratory tests (e.g., genetic tests for a gene signature associated with a specific chronic bronchopulmonary disease). A family history of a chronic bronchopulmonary disease, or exposure to risk factors for a chronic bronchopulmonary disease (e.g. long-term exposure to irritating gases or particulate matter; e.g., exposure to cigarette smoke) can also aid in determining if a subject is likely to have a chronic bronchopulmonary disease or in making a diagnosis of a chronic bronchopulmonary disease.

Subjects having an infectious bronchopulmonary disease can be identified by a physician using current methods of diagnosing an infectious bronchopulmonary disease. Symptoms and/or complications of an infectious bronchopulmonary disease which characterize these conditions and aid in diagnosis are well known in the art and include but are not limited to, chest pain when breathing or coughing; confusion or changes in mental awareness (e.g., in adults age 65 and older); cough, which can produce phlegm; fatigue; fever, sweating and shaking chills; lower than normal body temperature (e.g., in adults older than age 65 or people with weak immune systems); nausea, vomiting or diarrhea; and/or shortness of breath. Tests that may aid in a diagnosis of, e.g. an infectious bronchopulmonary disease include, but are not limited to, blood tests; blood cultures; oximetry; arterial blood gases; bronchoscopy; transtracheal mucus culture; lung biopsy; thoracentesis; and/or computed tomography (CT) scan. A family history of infectious bronchopulmonary disease, or exposure to risk factors for infectious bronchopulmonary disease (e.g. long-term exposure to irritating gases or particulate matter; exposure to cigarette smoke; autoimmunity; allergies; asthma; pregnancy) can also aid in determining if a subject is likely to have an infectious bronchopulmonary disease or in making a diagnosis of an infectious bronchopulmonary disease.

In some embodiments, the subject has been diagnosed with or is at risk of developing a bronchopulmonary disease. In some embodiments, the subject has been diagnosed with or is at risk of developing a chronic bronchopulmonary disease. In some embodiments, the chronic bronchopulmonary disease is selected from the group consisting of: chronic obstructive pulmonary disease (COPD), lung cancer, asthma, bronchiectasis, emphysema, cystic fibrosis (CF), bronchopulmonary dysplasia (BPD), acute respiratory disease syndrome (ARDS), idiopathic pulmonary fibrosis (IPF), pleural effusion (PE), pulmonary hypertension (e.g., pulmonary arterial hypertension, PAH), interstitial lung disease (ILD), and silicosis. In some embodiments, the lung cancer is small cell lung cancer (SCLC) or non-small cell lung cancer (NSCLC).

In some embodiments, the infectious bronchopulmonary disease is caused by or associated with an infectious agent selected from: adenovirus; coronavirus (e.g., common cold viruses; Severe Acute Respiratory Syndrome corona virus 1 (SARS-CoV-1); SARS-CoV-2; Middle East Respiratory Syndrome (MERS) CoV); influenza virus (e.g., flu); parainfluenza virus; parvovirus B19 (e.g., parvovirus B19; fifth disease); respiratory syncytial virus (RSV); rhinovirus (e.g., common cold); enterovirus (e.g., EV-D68); measles virus; rubella virus; varicella virus (e.g., chicken pox); Corynebacterium diphtheriae (e.g., diphtheria); Haemophilus influenzae (e.g., type b); Legionella pneumophila (e.g., Legionnaire's disease); Bordetella pertussis (pertussis); Mycobacterium tuberculosis (e.g., tuberculosis); Streptococcus species (e.g., Streptococcus pneumoniae, Streptococcus pyogenes; e.g., pneumonia); Pseudomonas species (e.g., Pseudomonas aeruginosa; e.g., lung infections, chronic recurrent respiratory infections); Escherichia coli (e.g., community-acquired pneumonia); Aspergillus species (e.g., Aspergillus fumigatus, Aspergillus flavus; Aspergillosis); Cryptococcus species (e.g., Cryptococcus neoformans, Cryptococcus gattii; pulmonary cryptococcosis); and Pneumocystis species (e.g., Pneumocystis jirovecii; pneumocystosis); see e.g., Dasaraju and Liu, “Chapter 93: Infections of the Respiratory System,” Medical Microbiology. 4th edition (1996) for non-limiting examples of infectious bronchopulmonary diseases.

In some embodiments, the chronic bronchopulmonary disease is chronic obstructive pulmonary disease (COPD). In some embodiments, the chronic bronchopulmonary disease is lung cancer. In some embodiments, the chronic bronchopulmonary disease is asthma. In some embodiments, the chronic bronchopulmonary disease is bronchiectasis. In some embodiments, the chronic bronchopulmonary disease is emphysema. In some embodiments, the chronic bronchopulmonary disease is cystic fibrosis (CF). In some embodiments, the chronic bronchopulmonary disease is bronchopulmonary dysplasia (BPD). In some embodiments, the chronic bronchopulmonary disease is acute respiratory disease syndrome (ARDS). In some embodiments, the chronic bronchopulmonary disease is idiopathic pulmonary fibrosis (IPF). In some embodiments, the chronic bronchopulmonary disease is interstitial lung disease (ILD). In some embodiments, the chronic bronchopulmonary disease is pleural effusion (PE). In some embodiments, the chronic bronchopulmonary disease is pulmonary hypertension (PAH). In some embodiments, the chronic bronchopulmonary disease is silicosis. In some embodiments, the chronic bronchopulmonary disease is small cell lung cancer (SCLC). In some embodiments, the chronic bronchopulmonary disease is non-small cell lung cancer (NSCLC). In some embodiments, the chronic bronchopulmonary disease is non-cystic fibrosis (CF) bronchiectasis.

In one aspect, described herein is a method of delivering a spray-dried pharmaceutical composition comprising a bacterial preparation to a subject, comprising: (a) obtaining an inhalation device for bronchopulmonary delivery comprising: (i) an inhaler; and (ii) a container containing a spray-dried pharmaceutical composition comprising a bacterial preparation; (b) activating the inhaler to cause aerosolization or dispersal of the spray-dried pharmaceutical composition; and (c) inhaling the aerosolized or dispersed spray-dried pharmaceutical composition.

In one aspect, described herein is a method of delivering a spray-dried pharmaceutical composition comprising a bacterial preparation to a subject, comprising: (a) obtaining an inhalation device for bronchopulmonary delivery comprising: (i) an inhaler; and (ii) a container containing a spray-dried pharmaceutical composition as described herein; (b) activating the inhaler to cause aerosolization or dispersal of the spray-dried pharmaceutical composition; and (c) inhaling the aerosolized or dispersed spray-dried pharmaceutical composition.

In one aspect, described herein is a method of delivering a spray-dried pharmaceutical composition comprising a bacterial preparation to a subject, comprising: (a) obtaining an inhalation device as described herein; (b) activating the inhaler to cause aerosolization or dispersal of the spray-dried pharmaceutical composition; and (c) inhaling the aerosolized or dispersed spray-dried pharmaceutical composition.

In some embodiments, the inhaler is a dry powder inhaler (DPI). In some embodiments, the inhaler is a metered dose inhaler (MDI). In some embodiments of any of the aspects, the inhaler is a soft mist inhaler (SMI). In some embodiments, the inhaler comprises: (a) a mouthpiece comprising an opening; and (b) means for aerosolizing or dispersing the spray-dried pharmaceutical composition in the container. In some embodiments, the inhaler has an inspiration flow rate of at least 15 L/min. In some embodiments, the inhaler has an inspiration flow rate of at least 15 L/min to at most 60 L/min. In some embodiments, the inhaler has an inspiration flow rate of at least 15 L/min to at most 120 L/min. In some embodiments, the inhaler has an inspiration flow rate of at least 15 L/min, at least 20 L/min, at least 25 L/min, at least 30 L/min, at least 35 L/min, at least 40 L/min, at least 45 L/min, at least 50 L/min, at least 55 L/min, at least 60 L/min, at least 70 L/min, at least 80 L/min, at least 90 L/min, at least 100 L/min, at least 110 L/min, or at most 120 L/min.

In some embodiments, at least 25% to at most 125.0% of the spray-dried pharmaceutical composition by mass is delivered to a target bronchopulmonary tissue. In some embodiments, at least 25% to at most 80.0% of the spray-dried pharmaceutical composition by mass is delivered to a target bronchopulmonary tissue. In some embodiments, at least 25% to at most 100.0% of the spray-dried pharmaceutical composition by mass is delivered to a target bronchopulmonary tissue. In some embodiments, at least 60.0% of the spray-dried pharmaceutical composition by mass is delivered to a target bronchopulmonary tissue. In some embodiments, at least 25%, at least 30%, at least 35%, at least 40%, at least 45%, at least 50%, at least 55%, at least 60%, at least 65%, at least 70%, at least 75%, at least 80%, at least 90%, at least 100%, at least 110%, at least 120%, or at most 125% of the spray-dried pharmaceutical composition by mass is delivered to a target bronchopulmonary tissue.

In some embodiments, at least 25% to at most 125% of the spray-dried pharmaceutical composition by mass is expelled from the inhalation device (e.g., inhaler). In some embodiments, at least 25% to at most 80.0% of the spray-dried pharmaceutical composition by mass is expelled from the inhalation device (e.g., inhaler). In some embodiments, at least 60.0% of the spray-dried pharmaceutical composition by mass is expelled from the inhalation device (e.g., inhaler). In some embodiments, at least 25%, at least 30%, at least 35%, at least 40%, at least 45%, at least 50%, at least 55%, at least 60%, at least 65%, at least 70%, at least 75%, at least 80%, at least 90%, at least 100%, at least 110%, at least 120%, or at most 125% of the spray-dried pharmaceutical composition by mass is expelled from the inhalation device (e.g., inhaler).

In some embodiments, the target bronchopulmonary tissue is the lungs, the trachea, the bronchi, the bronchioles, or the alveoli. In some embodiments, the target bronchopulmonary tissue is the lungs. In some embodiments, the target bronchopulmonary tissue is the trachea. In some embodiments, the target bronchopulmonary tissue is the bronchi. In some embodiments, the target bronchopulmonary tissue is the bronchioles. In some embodiments, the target bronchopulmonary tissue is alveoli.

In some embodiments, the dried particles have a median mass aerodynamic diameter (MMAD) of at least 0.5 μm to at most 10 μm, which can influence which bronchopulmonary regions the particles can reach. Particles from about 0.5 um to about 2.0 um are deposited in the alveoli and small airways, such as bronchioles, which is therapeutically useful. Particles from about 2.0 um to about 10.0 um are deposited in the tracheobronchial region of the airways, which is therapeutically useful. Particles less than 0.5 um are essentially exhaled, which is not therapeutically useful. Particles larger than about 10.0 um are retained in the oropharyngeal region and larynx due to impaction, which is not therapeutically useful. See e.g., Thakur et al., Chapter 22: Mucoadhesive drug delivery systems in respiratory diseases, in Targeting Chronic Inflammatory Lung Diseases Using Advanced Drug Delivery Systems, 2020, pp. 475-491; the content of which is incorporated herein by reference in its entirety.

In some embodiments, the spray-dried pharmaceutical composition is delivered from the bronchopulmonary tissue to a distal tissue site (i.e., non-pulmonary tissue) via the cardiovascular system or lymphatic system. Having bacterial preparations (e.g., live or non-living bacterial biotherapeutics) in the cardiovascular system or lymphatic system is not necessarily harmful to the subject, especially at low doses; for example, studies indicate that the blood exhibits a microbial signature; see e.g., Castillo et al., Front. Cell. Infect. Microbiol., 9: 148, 2019; the content of which is incorporated herein by reference in its entirety. In some embodiments, the distal tissue site (i.e., non-pulmonary tissue) is in the gastrointestinal system, cardiovascular system, lymphatic system, musculoskeletal system, nervous system, urinary system, reproductive system, endocrine system, or integumentary system. In some embodiments, the distal tissue site (i.e., non-pulmonary tissue) is connective tissue, epithelial tissue, muscle tissue, or nervous tissue.

The compositions and methods described herein can be administered to a subject having or diagnosed as having a bronchopulmonary disease, e.g., a chronic bronchopulmonary disease. In some embodiments, the methods described herein comprise administering an effective amount of a composition described herein, e.g., a spray-dried pharmaceutical composition (e.g., comprising a bacterial preparation) as described herein to a subject in order to alleviate a symptom of a chronic bronchopulmonary disease. As used herein, “alleviating a symptom of a chronic bronchopulmonary disease” is ameliorating any condition or symptom associated with the chronic bronchopulmonary disease. As compared with an equivalent untreated control, such reduction is by at least 5%, 10%, 20%, 40%, 50%, 60%, 80%, 90%, 95%, 99% or more as measured by any standard technique.

Compared with other routes of administration, inhalation offers a number of unique benefits (see e.g., Borghardt et al. 2018, the content of which is incorporated herein by reference in its entirety). Inhalation delivers bacterial preparations directly to the lung tissue, resulting in high pulmonary concentrations and low systemic concentrations, and can be associated with minimal systemic side effects. Inhaled formulations containing bacterial preparations can be created through a number of processing routes, including spray drying to create a dry powder inhaled formulation, compounding to create a nebulizable formulation, or compounding and pressurizing in metered dose inhalers. Each of these routes of administration offer benefits. Dry powder formulations are composed of engineered particles with specific size distributions, allowing for more precisely tuned lung deposition, aerodynamic properties, long-term stability, and relative ease of dosing in patients with normal inhalation capacity. Nebulized solutions containing active pharmaceutical ingredients are well-suited for infant or adolescent lungs, as well as patients with weaker lung capacity. Nebulized solutions can face stability challenges and have size limitations that can prevent aerosolization during dosing.

Metered dose inhalers are common in the market for bronchodilators and inhaled corticosteroids, but typically require a pressurized container and a chemical propellant to work effectively. Some of these propellants have unknown side-effects, and sustained pressure on a drug-containing solution may have stability challenges, especially when formulating with more sensitive biotherapeutics. While propellants and high pressures are not generally conducive to maintaining viability of bacteria, MDIs can be used to deliver for non-viable bacteria (e.g., heat-killed bacteria), bacterial extracts, and/or bacterial products.

The term “effective amount” as used herein refers to the amount of a spray-dried pharmaceutical composition (e.g., comprising a bacterial preparation) as described herein needed to alleviate at least one or more symptom of the disease or disorder, and relates to a sufficient amount of pharmacological composition to provide the desired effect. The term “therapeutically effective amount” therefore refers to an amount of a spray-dried pharmaceutical composition (e.g., comprising a bacterial preparation) as described herein that is sufficient to provide a particular anti-bronchopulmonary disease effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

In some embodiments, effective amounts of the spray-dried inhalable biotherapeutics described herein can be determined using biodistribution and/or translocation studies to determine where in the body the administered bacteria travel to and/or deposit, dynamics studies of how fast the bacteria clear from the target tissue and whether the bacteria engraft or colonize the target tissue, and/or studies to assess changes in metabolic output (e.g., of the subject) as a result of bacteria-derived byproducts. Effective amounts, toxicity, and therapeutic efficacy can also be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a metabolite produced by an administered bacterium, which achieves a half-maximal inhibition of symptoms) as determined in cell culture, or in an appropriate animal model. Levels in plasma can be measured, for example of metabolite(s) produced by an administered bacterium, by high performance liquid chromatography. The effects of any particular dosage can be monitored by a suitable bioassay, e.g., assay for pH or apoptosis, among others. The dosage can be determined by a physician and adjusted, as necessary, to suit observed effects of the treatment.

In some embodiments of any of the aspects, the spray-dried biotherapeutic matrix composition (e.g., comprising a bacterial preparation) described herein is administered as a monotherapy, e.g., another treatment for the chronic bronchopulmonary disease is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include a cancer therapy selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gammalI and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE@doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003). In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In some embodiments, the spray-dried biotherapeutic matrix composition is co-administered with the at least one additional therapeutic treatment or therapeutic intervention for a chronic bronchopulmonary disorder.

In some embodiments, the chronic bronchopulmonary disorder is chronic obstructive pulmonary disease (COPD), and the at least one additional therapeutic treatment or therapeutic intervention is: smoking cessation; bronchodilators; inhaled steroids; oral steroids; roflumilast (DALIRESP; a phosphodiesterase-4 inhibitor); theophylline (ELIXOPHYLLIN, THEO-24, THEOCHRON); antibiotics; oxygen therapy; pulmonary rehabilitation (e.g., education, breathing exercises, nutrition advice, and/or counseling); in-home noninvasive ventilation therapy (e.g., bilevel positive airway pressure (BiPAP)); and/or surgery (e.g., lung volume reduction surgery; bullectomy; or lung transplant).

In some embodiments, the chronic bronchopulmonary disorder is lung cancer, and the at least one additional therapeutic treatment or therapeutic intervention is: surgery (e.g., wedge resection; segmental resection; lobectomy; or pneumonectomy); radiation therapy; chemotherapy; stereotactic body radiotherapy; gene-targeted drug therapy; or immunotherapy.

In some embodiments, the chronic bronchopulmonary disorder is asthma, and the at least one additional therapeutic treatment or therapeutic intervention is: bronchodilators; inhaled corticosteroids (e.g., fluticasone propionate (FLOVENT HFA, FLOVENT DISKUS, XHANCE), budesonide (PULMICORT FLEXHALER, PULMICORT RESPULES, RHINOCORT), ciclesonide (ALVESCO), beclomethasone (QVAR REDIHALER), mometasone (ASMANEX HFA, ASMANEX TWISTHALER) and fluticasone furoate (ARNUITY ELLIPTA); leukotriene modifiers. (e.g., montelukast (SINGULAIR), zafirlukast (ACCOLATE) and zileuton (ZYFLO); combination treatments of a long-acting beta agonist and a corticosteroid (e.g., fluticasone-salmeterol (ADVAIR HFA, AIRDUO DIGIHALER), budesonide-formoterol (SYMBICORT), formoterol-mometasone (DULERA) and fluticasone furoate-vilanterol (BREO ELLIPTA); theophylline (ELIXOPHYLLIN, THEO-24, THEOCHRON); short-acting beta agonists (e.g., albuterol (PROAIR HFA, VENTOLIN HFA) and levalbuterol (XOPENEX, XOPENEX HFA); anticholinergic agents (e.g., ipratropium (ATROVENT HFA) and tiotropium (SPIRIVA, SPIRIVA RESPIMAT); oral and intravenous corticosteroids (e.g., prednisone (PREDNISONE INTENSOL, RAYOS) and methylprednisolone (MEDROL, DEPO-MEDROL, SOLU-MEDROL); allergy shots (e.g., injections with a specific allergen to gradually reduce immune system reaction); allergy biologics (e.g., omalizumab (XOLAIR), mepolizumab (NUCALA), dupilumab (DUPIXENT), reslizumab (CINQAIR) or benralizumab (FASENRA)); or bronchial thermoplasty.

In some embodiments, the chronic bronchopulmonary disorder is bronchiectasis, and the at least one additional therapeutic treatment or therapeutic intervention is: antibiotics; macrolides; mucus thinning medication (e.g., guaifenesin (MUCINEX)); airway clearance devices (e.g., positive expiratory pressure (PEP) devices; percussive, wearable devices such as percussive vests); or chest physical therapy (e.g., chest clapping).

In some embodiments, the chronic bronchopulmonary disorder is emphysema, and the at least one additional therapeutic treatment or therapeutic intervention is: bronchodilators; inhaled steroids; antibiotics; pulmonary rehabilitation (e.g., breathing exercises); nutrition therapy; supplemental oxygen; smoking cessation; or surgery (e.g., lung volume reduction surgery; lung transplant).

In some embodiments, the chronic bronchopulmonary disorder is cystic fibrosis (CF), and the at least one additional therapeutic treatment or therapeutic intervention is: cystic fibrosis transmembrane conductance regulator (CFTR) modulators (e.g., a combination medication containing elexacaftor, ivacaftor and tezacaftor (TRIKAFTA); a combination medication containing tezacaftor and ivacaftor (SYMDEKO); a combination medication containing lumacaftor and ivacaftor (ORKAMBI); ivacaftor (KALYDECO); airway clearance devices (e.g., positive expiratory pressure (PEP) devices; percussive, wearable devices such as percussive vests); chest physical therapy (e.g., chest clapping); pulmonary rehabilitation (e.g., breathing exercises); nasal and sinus surgery; oxygen therapy; noninvasive ventilation; feeding tube; bowel surgery; lung transplant; or liver transplant.

In some embodiments, the chronic bronchopulmonary disorder is bronchopulmonary dysplasia (BPD), and the at least one additional therapeutic treatment or therapeutic intervention is: diuretics (e.g., to decrease the amount of fluid in and around the alveoli); bronchodilators; corticosteroids; cardiac medications; or respiratory syncytial virus (RSV) immunization to prevent or reduce respiratory tract infections.

In some embodiments, the chronic bronchopulmonary disorder is acute respiratory disease syndrome (ARDS), and the at least one additional therapeutic treatment or therapeutic intervention is: Supplemental oxygen; Mechanical ventilation; intravenous fluids; smoking cessation; antibiotics; analgesics; anti-inflammatories; blood thinners; gastric reflux drugs (e.g., esomeprazole (NEXIUM)); or sedation.

In some embodiments, the chronic bronchopulmonary disorder is idiopathic pulmonary fibrosis (IPF), and the at least one additional therapeutic treatment or therapeutic intervention is: anti-fibrotics (e.g., Nintedanib (OFEV) or pirfenidone (ESBRIET)); corticosteroids (e.g., prednisone); immunosuppressive drugs (e.g., azathioprine (IMURAN), cyclophosphamide (CYTOXAN), or mycophenolate mofetil (CELLCEPT)); oral or spray antioxidant (e.g., N-acetylcysteine, NAC (MUCOMYST)); oxygen therapy; or pulmonary rehabilitation (e.g., breathing exercises).

In some embodiments, the chronic bronchopulmonary disorder is interstitial lung disease (ILD), and the at least one additional therapeutic treatment or therapeutic intervention is: a corticosteroid (e.g., prednisone); an immunosuppressant; a medication that slows the progression of idiopathic pulmonary fibrosis, such as pirfenidone (ESBRIET) or nintedanib (OFEV); H-2-receptor antagonists or proton pump inhibitors such as lansoprazole (PREVACID 24HR), omeprazole (Prilosec OTC) or pantoprazole (PROTONIX), e.g., for symptoms of gastroesophageal reflux disease (GERD) that affect the majority of people with idiopathic pulmonary fibrosis; oxygen therapy; pulmonary rehabilitation; and/or surgery, such as lung transplantation.

In some embodiments, the chronic bronchopulmonary disorder is pleural effusion (PE), and the at least one additional therapeutic treatment or therapeutic intervention is: a diuretic; chemotherapy; radiation therapy; therapeutic thoracentesis; tube thoracostomy; a sclerosing agent (e.g., talc, doxycycline, and tetracycline); and/or surgery, such as video-assisted thoracoscopic surgery (VATS) or thoracotomy.

In some embodiments, the chronic bronchopulmonary disorder is pulmonary hypertension (PAH), and the at least one additional therapeutic treatment or therapeutic intervention is: a vasodilator; a guanylate cyclase (GSC) stimulator such as riociguat (ADEMPAS); an endothelin receptor antagonist such as bosentan (TRACLEER), macitentan (OPSUMIT) or ambrisentan (LETAIRIS); phosphodiesterase 5 (PDE5) inhibitors such as sildenafil (REVATIO, VIAGRA) or tadalafil (ADCIRCA, CIALIS, ALYQ); a calcium channel blocker such as amlodipine (NORVASC), diltiazem (CARDIZEM, TIAZAC) or nifedipine (PROCARDIA); an anticoagulant such as warfarin (JANTOVEN); digoxin (LANOXIN); diuretics; oxygen therapy; and/or surgery, such as atrial septostomy or lung or heart-lung transplant.

In some embodiments, the chronic bronchopulmonary disorder is silicosis, and the at least one additional therapeutic treatment or therapeutic intervention is: a bronchodilator; cessation of smoking; supplemental oxygen; pulmonary rehabilitation; and/or surgery, such as lung transplantation.

In some embodiments, the at least one additional therapeutic treatment or therapeutic intervention for the infectious bronchopulmonary disorder is an antiviral, an antibiotic, or an antifungal, e.g., non-limiting examples of which are listed above.

In some embodiments, the methods described herein can further comprise administering an antibiotic to the subject, e.g. as part of a combinatorial therapy. In some embodiments, the antibiotic is administered before, during, or after administration of the spray-dried biotherapeutic matrix composition. In some embodiments, the antibiotic is administered to kill or decrease the growth of bacteria naturally growing in the target tissue, e.g., in order to allow the bacteria of the spray-dried biotherapeutic matrix composition to grow and/or become established in the target tissue. In some embodiments, the antibiotic is administered to kill or decrease the growth of bacteria from the spray-dried biotherapeutic matrix composition, e.g., after a sufficient amount of time or growth of such bacteria. In some embodiments, bacteria from the spray-dried biotherapeutic matrix composition are resistant to the administered antibiotics (e.g., when the antibiotic is administered to kill or reduce the growth of the natural bacteria of the target tissue). In some embodiments, bacteria from the spray-dried biotherapeutic matrix composition are not resistant (i.e., susceptible) to the administered antibiotics (e.g., when the antibiotic is administered to kill or reduce the growth of the bacteria from the spray-dried biotherapeutic matrix composition).

In some embodiments of any of the aspects, the antibiotic is selected from amikacin; aztreonam; cefepime; cefoxitin; ciprofloxacin; levofloxacin; metronidazole; trim/sulfa; trimethoprim; vancomycin; extended-spectrum beta-lactamases (ESBL) plazomicin; fosfomycin; ceftazidime; or ofloxacin.

In some embodiments of any of the aspects, the antibiotic can be selected from aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof.

Some exemplary specific antibiotics include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefietazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

In certain embodiments, an effective dose of a composition comprising a bacterial preparation as described herein can be administered to a patient once. In certain embodiments, an effective dose of a composition comprising a bacterial preparation can be administered to a patient repeatedly. For systemic administration, subjects can be administered a therapeutic amount of a composition comprising a bacterial preparation, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of the chronic bronchopulmonary disease by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the bacterial preparation. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a bacterial preparation can be administered over a period of time, such as over a 5 minute, 10 minute, 15 minute, 20 minute, or 25 minute period.

The dosage ranges for the administration of a spray-dried biotherapeutic matrix composition (e.g., comprising a bacterial preparation), according to the methods described herein depend upon, for example, the form of the bacterial preparation, its potency, and the extent to which symptoms, markers, or indicators of a condition described herein are desired to be reduced. The dosage should not be so large as to cause adverse side effects, such as infection or sepsis. Generally, the dosage will vary with the age, condition, and sex of the patient and can be determined by one of skill in the art. The dosage can also be adjusted by the individual physician in the event of any complication.

The efficacy of the spray-dried biotherapeutic matrix composition (e.g., comprising a bacterial preparation) in, e.g. the treatment of a condition described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms; or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of chronic obstructive pulmonary disease (COPD), lung cancer, asthma, bronchiectasis, emphysema, cystic fibrosis (CF), bronchopulmonary dysplasia (BPD), acute respiratory disease syndrome (ARDS), or idiopathic pulmonary fibrosis (IPF). When using an experimental animal model, efficacy of treatment is evidenced when a statistically significant change in a marker is observed.

In vitro and animal model assays allow the assessment of a given dose of a spray-dried biotherapeutic matrix composition (e.g., comprising a bacterial preparation). By way of non-limiting example, the effects of a dose of a spray-dried biotherapeutic matrix composition (e.g., comprising a bacterial preparation) can be assessed in a dysbiosis model of lung epithelial cells, a murine model of dysbiosis, or in A549 non-small cell lung cancer (NSCLC) adenocarcinoma cells.

### Treatment Methods

The compositions described herein can be administered to a subject in need thereof, for instance for the treatment of a bronchopulmonary disease, including, but not limited to a chronic bronchopulmonary disease. In some embodiments, the method of treatment can comprise first diagnosing a subject or patient who can benefit from treatment by a composition described herein. In some embodiments, the method further comprises administering to the patient a composition as described herein.

In one aspect, described herein is a method of treating a subject in need thereof comprising administering through inhalation an effective dose of a spray-dried biotherapeutic matrix composition comprising a bacterial preparation. In one aspect, described herein is a method of treating a subject in need thereof comprising administering through inhalation an effective dose of a spray-dried biotherapeutic matrix composition as described herein.

In some embodiments, the spray-dried biotherapeutic matrix composition (e.g., the bacterial preparation in the spray-dried biotherapeutic matrix composition) reduces neutrophilic inflammation in a target tissue. In some embodiments, the spray-dried biotherapeutic matrix composition reduces neutrophilic inflammation in a target tissue by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% or more.

In some embodiments, administration of the spray-dried biotherapeutic matrix composition results in production of an acid in the target tissue of the subject. In some embodiments, administration of the spray-dried biotherapeutic matrix composition results in lactic acid (LA) production in the target tissue. In some embodiments, administration of the spray-dried biotherapeutic matrix composition results in lactic acid production in a bronchopulmonary target tissue (e.g., the lungs). In some embodiments, increased lactic acid production can be associated with decreased neutrophilic inflammation in a target tissue. In some embodiments, the lactic acid is produced by viable bacteria in the administered spray-dried biotherapeutic matrix composition. In some embodiments, the lactic acid is produced by cells of the subject, e.g., triggered by at least one bacterial preparation (e.g., viable or non-viable bacteria or components thereof) in the administered spray-dried biotherapeutic matrix composition.

In some embodiments, at least 4 nmol/mL of lactic acid is produced in a target tissue following administration (e.g., at least 2 hours, at least 8 hours, at least 12 hours, or at least 16 hours following administration) of the spray-dried biotherapeutic matrix composition (see e.g., FIG. 4C). In some embodiments, at least 10 nmol/mL of lactic acid is produced in a target tissue following administration of the spray-dried biotherapeutic matrix composition. In some embodiments, at least 9 nmol/mL, at least 9.5 nmol/mL, at least 10 nmol/mL, at least 10.5 nmol/mL, at least 11 nmol/mL, at least 11.5 nmol/mL, at least 12 nmol/mL, at least 12.5 nmol/mL, at least 13 nmol/mL, at least 13.5 nmol/mL, at least 14 nmol/mL, at least 14.5 nmol/mL, at least 15 nmol/mL or more of lactic acid is produced in a target tissue following administration of the spray-dried biotherapeutic matrix composition. In some embodiments, the spray-dried biotherapeutic matrix composition (e.g., the bacterial preparation in the spray-dried biotherapeutic matrix composition) increases lactic acid concentration in a target tissue by at least 100%. In some embodiments, the spray-dried biotherapeutic matrix composition increases lactic acid concentration in a target tissue by at least about 25%. In some embodiments, the spray-dried biotherapeutic matrix composition increases lactic acid concentration in a target tissue by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 150%, at least about 200%, or more.

In some embodiments, the target tissue is a target bronchopulmonary tissue. In some embodiments, the target bronchopulmonary tissue is the lungs, the trachea, the bronchi, the bronchioles, and/or the alveoli. In some embodiments, the target tissue is a distal tissue site from the lungs delivered via the cardiovascular system or lymphatic system.

In some embodiments, the subject has been diagnosed with or is at risk of developing a chronic or infectious bronchopulmonary disease. In some embodiments, the subject has been diagnosed with or is at risk of developing a chronic bronchopulmonary disease. In some embodiments, the chronic bronchopulmonary disease is selected from the group consisting of: chronic obstructive pulmonary disease (COPD), lung cancer, asthma, bronchiectasis, emphysema, cystic fibrosis (CF), bronchopulmonary dysplasia (BPD), acute respiratory disease syndrome (ARDS), and idiopathic pulmonary fibrosis (IPF) interstitial lung disease (ILD), pleural effusion (PE), pulmonary hypertension (PAH), and silicosis. In some embodiments, the lung cancer is small cell lung cancer (SCLC) or non-small cell lung cancer (NSCLC).

In some embodiments, the subject has been diagnosed with or is at risk of developing an infectious bronchopulmonary disease. In some embodiments, the infectious bronchopulmonary disease is caused by or associated with an infectious agent selected from: adenovirus; coronavirus (e.g., common cold viruses; Severe Acute Respiratory Syndrome corona virus 1 (SARS-CoV-1); SARS-CoV-2; Middle East Respiratory Syndrome (MERS) CoV); influenza virus (e.g., flu); parainfluenza virus; parvovirus B19 (e.g., parvovirus B19; fifth disease); respiratory syncytial virus (RSV); rhinovirus (e.g., common cold); enterovirus (e.g., EV-D68); measles virus; rubella virus; varicella virus (e.g., chicken pox); Corynebacterium diphtheriae (e.g., diphtheria); Haemophilus influenzae (e.g., type b); Legionella pneumophila (e.g., Legionnaire's disease); Bordetella pertussis (pertussis); Mycobacterium tuberculosis (e.g., tuberculosis); Streptococcus species (e.g., Streptococcus pneumoniae, Streptococcus pyogenes; e.g., pneumonia); Pseudomonas species (e.g., Pseudomonas aeruginosa; e.g., lung infections, chronic recurrent respiratory infections); Escherichia coli (e.g., community-acquired pneumonia); Aspergillus species (e.g., Aspergillus fumigatus, Aspergillus flavus; Aspergillosis); Cryptococcus species (e.g., Cryptococcus neoformans, Cryptococcus gattii; pulmonary cryptococcosis); and Pneumocystis species (e.g., Pneumocystis jirovecii; pneumocystosis).

In some embodiments, the effective dose of the spray-dried biotherapeutic matrix composition is at least 200×10⁶ CFU of bacteria per unit dose. In some embodiments, the effective dose of the spray-dried biotherapeutic matrix composition is at least 10¹ CFU, at least 10² CFU, at least 10³ CFU, at least 10⁴ CFU, at least 10⁵ CFU, at least 10⁶ CFU, at least 10⁷ CFU, at least 10⁸ CFU, at least 10⁹ CFU, at least 10¹⁰ CFU, at least 10¹¹ CFU, or at least 10¹² CFU of bacteria per unit dose. In some embodiments, the effective dose of the spray-dried biotherapeutic matrix composition is at least 200×10⁶ CFU of viable bacteria per unit dose. In some embodiments, the effective dose of the spray-dried biotherapeutic matrix composition is at least 10¹ CFU, at least 10² CFU, at least 10³ CFU, at least 10⁴ CFU, at least 10⁵ CFU, at least 10⁶ CFU, at least 10⁷ CFU, at least 10⁸ CFU, at least 10⁹ CFU, at least 10¹⁰ CFU, at least 10¹¹ CFU, or at least 10¹² CFU of viable bacteria per unit dose.

In some embodiments, the spray-dried biotherapeutic matrix composition formulated for administration by inhalation is co-administered with at least one additional therapeutic for a chronic or infectious bronchopulmonary disorder. In some embodiments, the at least one additional therapeutic is an anti-inflammatory, an antimicrobial, an antiviral, an antifungal, a vasodilator, or a bronchodilator, as described further herein. In some embodiments, the spray-dried biotherapeutic matrix composition comprises the bacterial preparation and the at least one additional therapeutic in the same composition or unit dosage. As a non-limiting example, the bacterial preparation and the at least one additional therapeutic can be spray-dried together and formulated for administration together, e.g., in a single capsule. In some embodiments, the bacterial preparation and the at least one additional therapeutic are each spray-dried separately and formulated for administration, e.g., in the same or different capsules. In some embodiments, the bacterial preparation and the at least one additional therapeutic co-administered using a combination delivery device, such as an inhaler device that administers multiple different formulations at once.

In some embodiments, the spray-dried biotherapeutic matrix composition is administered in conjunction with a standard of care for the chronic or infectious bronchopulmonary disease, as known to a person of skill in the art. As used herein, the term “standard of care” refers to the level at which the average, prudent provider in a given community would practice, e.g., in treating a given indication.

As a non-limiting example, the COPD standard of care changes based on GOLD stage classification of severity, but can include a variety of therapies combining anti-inflammatory action (w.g., inhaled steroids) and symptom relief (e.g., long-acting beta-agonist (LABA), long-acting muscarinic antagonist (LAMA)). Pulmonary fibrosis standard of care is typically at least one of two antifibrotics: nintedanib and pirfenidone. Bronchopulmonary dysplasia standard of care includes a variety of treatments based on the risk profile of the patient including supplemental oxygen, surfactants, and bronchodilators. See e.g., Safka et al., Chronic Obstr Pulm Dis. 2017; 4(1): 45-55 (see e.g., Table 1 of Safka); 2022 Gold Reports, available on the world wide web at goldcopd.org/2022-gold-reports (see e.g., Page 49-57; Page 49 Table 3.3; Page 51 Table 3.4; Page 53 Table 3.5 of the Gold Reports); Maher et al. “Respiratory Research volume 20, Article number: 205 (2019). Non-limiting examples of additional therapeutic treatment or therapeutic intervention for specific chronic or infectious bronchopulmonary diseases are described further herein.

In some embodiments, the spray-dried biotherapeutic matrix composition is administered to treat, mitigate, or prevent a central nervous system (CNS) disease, non-limiting examples of which include Parkinson's disease, multiple sclerosis (MS), and migraines. It is noted that among indicators of the lung-brain axis is the discovery that dysregulation of the lung microbiota significantly influenced the susceptibility of rats to developing autoimmune disease of the CNS. See, e.g., Hosang et al., Nature 603: 138-144 (2022), which reports that local treatment with neomycin, which shifts the lung microbiota towards LPS-enriched phyla, induced a type I interferon-primed state in brain-resident microglial cells, impairing autoimmune stimulation by type II interferons, decreasing proinflammatory response and immune cell recruitment. In that study, suppression of LPS-producing phyla in the lung aggravated disease, but the addition of LPS-enriched phyla or LPS itself recapitulated the effect of neomycin. Thus, inhaled delivery of microbial products or microbes as described herein can influence the treatment, mitigation or prevention of CNS diseases or disorders. See e.g., Noymer et al. (2011) Ther Deliv 2 (9):1125-1140; Abdou et al. (2019) Drug Deliv 26 (1):689-699; Hosang et al. Nature 603: 138-144 (2022); the contents of each of which are incorporated herein by reference in their entireties.

### Unit Dosage Forms

In one aspect, described herein is a unit dosage form comprising a spray-dried biotherapeutic matrix composition as described herein. In some embodiments, a single unit dosage is one capsule comprising a spray-dried biotherapeutic matrix composition as described herein. In some embodiments, a single unit dosage is a plurality of (e.g., 1, 2, 3, 4, 5, or more) capsules, each comprising a spray-dried biotherapeutic matrix composition as described herein. In some embodiments, the unit dosage is administered (e.g., inhaled) during one inhalation (e.g., in less than 10, 20, or 30 seconds). In some embodiments, the unit dosage is administered (e.g., inhaled) over a period of time (e.g., greater than 30, 60, or 120 seconds) and/or administered (e.g., inhaled) with multiple usages of an administering inhaler.

In some embodiments, the unit dosage comprising a spray-dried biotherapeutic matrix composition as described herein results in about 100×10⁶ CFU of bacteria being delivered from a 30 mg capsule to the patient's lungs. In some embodiments, the unit dosage comprising a spray-dried biotherapeutic matrix composition as described herein results in about 200×10⁶ CFU of bacteria being delivered from a 30 mg capsule to the patient's lungs. This is a reasonable dose case, considering doses of bacteria spores to the gut for treatment of C. difficile consisted of 4 doses per day of 1×10⁸ CFU (400×10⁶ CFU total) per day; see e.g., McGovern et al. Clin Infect Dis. 2021; 72(12):2132-2140, the content of which is incorporated by reference herein in its entirety. Once the final dry powder is produced, it can be filled into capsules for delivery by dry powder inhaler. The dry powder blend itself can go through a number of process steps that would result in a uniform blend and strong solid dosage form.

In one aspect, described herein is a unit dosage form comprising at least 1 mg to at most 50 mg of a spray-dried biotherapeutic matrix composition comprising a bacterial preparation. In some embodiments, the unit dosage form comprises at least 7.5 mg bacterial preparation per unit dose. In some embodiments, the unit dosage form comprises at least 15 mg bacterial preparation per unit dose. In some embodiments, the unit dosage form comprises at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, or at least 50 mg bacterial preparation per unit dose. In some embodiments, the unit dosage form comprises at most 1 mg, at most 2 mg, at most 3 mg, at most 4 mg, at most 5 mg, at most 10 mg, at most 15 mg, at most 20 mg, at most 25 mg, at most 30 mg, at most 35 mg, at most 40 mg, at most 45 mg, or at most 50 mg bacterial preparation per unit dose. In some embodiments, the unit dosage can comprise a low amount of bacterial preparation or spray-dried biotherapeutic matrix composition when there is a high concentration of bacteria. In some embodiments, the unit dosage can comprise a high amount of bacterial preparation or spray-dried biotherapeutic matrix composition when there is a low concentration of bacteria in the bacterial preparation.

In one aspect, described herein is a unit dosage form comprising at least 1 mg to at most 50 mg of a spray-dried biotherapeutic matrix composition as described herein. In one aspect, described herein is a unit dosage form comprising at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, or at least 50 mg of a spray-dried biotherapeutic matrix composition as described herein. In one aspect, described herein is a unit dosage form comprising at most 1 mg, at most 2 mg, at most 3 mg, at most 4 mg, at most 5 mg, at most 10 mg, at most 15 mg, at most 20 mg, at most 25 mg, at most 30 mg, at most 35 mg, at most 40 mg, at most 45 mg, or at most 50 mg of a spray-dried biotherapeutic matrix composition as described herein.

In one aspect, described herein is a unit dosage form comprising at least 1 mg to at most 50 mg of the spray-dried biotherapeutic matrix composition prepared by the methods as described herein. In one aspect, described herein is a unit dosage form comprising at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, or at least 50 mg of the spray-dried biotherapeutic matrix composition prepared by the methods as described herein. In one aspect, described herein is a unit dosage form comprising at most 1 mg, at most 2 mg, at most 3 mg, at most 4 mg, at most 5 mg, at most 10 mg, at most 15 mg, at most 20 mg, at most 25 mg, at most 30 mg, at most 35 mg, at most 40 mg, at most 45 mg, or at most 50 mg of the spray-dried biotherapeutic matrix composition prepared by the methods as described herein.

In one aspect, described herein is a unit dosage form comprising at least 1 mg to at most 50 mg of a spray-dried biotherapeutic matrix composition comprising at least 10⁴ CFU bacteria per unit dose. In one aspect, described herein is a unit dosage form comprising at least 1 mg to at most 50 mg of a spray-dried biotherapeutic matrix composition comprising at least 100×10⁴ CFU bacteria per unit dose. In one aspect, described herein is a unit dosage form comprising at least 1 mg to at most 50 mg of a spray-dried biotherapeutic matrix composition comprising at least 100×10⁶ CFU bacteria per unit dose. In one aspect, described herein is a unit dosage form comprising at least 1 mg to at most 50 mg of a spray-dried biotherapeutic matrix composition comprising at least 200×10⁶ CFU bacteria per unit dose. In one aspect, described herein is a unit dosage form comprising at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, or at least 50 mg of a spray-dried biotherapeutic matrix composition comprising at least 200×10⁶ CFU bacteria per unit dose. In one aspect, described herein is a unit dosage form comprising at most 1 mg, at most 2 mg, at most 3 mg, at most 4 mg, at most 5 mg, at most 10 mg, at most 15 mg, at most 20 mg, at most 25 mg, at most 30 mg, at most 35 mg, at most 40 mg, at most 45 mg, or at most 50 mg of a spray-dried biotherapeutic matrix composition comprising at most 200×10⁶ CFU bacteria per unit dose.

In one aspect, described herein is a unit dosage form comprising at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, or at least 50 mg of a spray-dried biotherapeutic matrix composition comprising at least 100×10⁶ CFU bacteria per unit dose. In one aspect, described herein is a unit dosage form comprising at most 1 mg, at most 2 mg, at most 3 mg, at most 4 mg, at most 5 mg, at most 10 mg, at most 15 mg, at most 20 mg, at most 25 mg, at most 30 mg, at most 35 mg, at most 40 mg, at most 45 mg, or at most 50 mg of a spray-dried biotherapeutic matrix composition comprising at most 100×10⁶ CFU bacteria per unit dose.

In some embodiments, the dosage is at least 30 mg spray-dried biotherapeutic matrix composition. In some embodiments, the dosage is at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg spray-dried biotherapeutic matrix composition. In some embodiments, the dosage is at most 1 mg, at most 2 mg, at most 3 mg, at most 4 mg, at most 5 mg, at most 10 mg, at most 15 mg, at most 20 mg, at most 25 mg, at most 30 mg, at most 35 mg, at most 40 mg, at most 45 mg, at most 50 mg, at most 55 mg, at most 60 mg, at most 65 mg, at most 70 mg, at most 75 mg, at most 80 mg, at most 85 mg, at most 90 mg, at most 95 mg, or at most 100 mg spray-dried biotherapeutic matrix composition.

In some embodiments, the dosage comprises at least 10⁸ CFU of bacteria per unit dose. In some embodiments, the dosage comprises at least 200×10⁶ CFU of bacteria per unit dose. In some embodiments, the dosage comprises at least 200×10⁴ CFU of bacteria per unit dose. In some embodiments, the dosage comprises at least 10¹ CFU, at least 10² CFU, at least 10³ CFU, at least 10⁴ CFU, at least 10⁵ CFU, at least 10⁶ CFU, at least 10⁷ CFU, at least 10⁸ CFU, at least 10⁹ CFU, at least 10¹⁰ CFU, at least 10¹¹ CFU, or at least 10¹² CFU of bacteria per unit dose. In some embodiments, the dosage comprises at least 10⁴ CFU of viable bacteria per unit dose. In some embodiments, the dosage comprises at least 200×10⁶ CFU of viable bacteria per unit dose. In some embodiments, the dosage comprises at least 10¹ CFU, at least 10² CFU, at least 10³ CFU, at least 10⁴ CFU, at least 10⁵ CFU, at least 10⁶ CFU, at least 10⁷ CFU, at least 10⁸ CFU, at least 10⁹ CFU, at least 10¹⁰ CFU, at least 10¹¹ CFU, or at least 10¹² CFU of viable bacteria per unit dose. In some embodiments, the dosage comprises at most 10¹ CFU, at most 10² CFU, at most 10³ CFU, at most 10⁴ CFU, at most 10⁵ CFU, at most 10⁶ CFU, at most 10⁷ CFU, at most 10⁸ CFU, at most 10⁹ CFU, at most 10¹⁰ CFU, at most 10¹¹ CFU, or at most 10¹² CFU of bacteria per unit dose. In some embodiments, the dosage comprises at most 10¹¹ CFU of viable bacteria per unit dose. In some embodiments, the dosage comprises at most 200×10⁶ CFU of viable bacteria per unit dose. In some embodiments, the dosage comprises at most 10¹ CFU, at most 10² CFU, at most 10³ CFU, at most 10⁴ CFU, at most 10⁵ CFU, at most 10⁶ CFU, at most 10⁷ CFU, at most 10⁸ CFU, at most 10⁹ CFU, at most 10¹⁰ CFU, at most 10¹¹ CFU, or at most 10¹² CFU of viable bacteria per unit dose.

In some embodiments, the unit dosage comprises at least 0.5% bacterial preparation by dry weight. In some embodiments, the unit dosage comprises at least 25% bacterial preparation by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more bacterial preparation by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% bacterial preparation by dry weight.

In some embodiments, the unit dosage comprises at least 0.5% excipient(s) by dry weight. In some embodiments, the unit dosage comprises at least 70% excipient(s) by dry weight. In some embodiments, the unit dosage comprises at least 73% excipient(s) by dry weight. In some embodiments, the unit dosage comprises at least 73.4% excipient(s) by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more excipient(s) by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% excipient(s) by dry weight.

In some embodiments, the unit dosage comprises at least 0.5% of a first excipient, a second excipient, and/or a third excipient by dry weight. In some embodiments, the unit dosage comprises at least 5% of a first excipient, a second excipient, and/or a third excipient by dry weight. In some embodiments, the unit dosage comprises at least 30% of a first excipient, a second excipient, and/or a third excipient by dry weight. In some embodiments, the unit dosage comprises at least 34% of a first excipient, a second excipient, and/or a third excipient by dry weight. In some embodiments, the unit dosage comprises at least 34.2% of a first excipient, a second excipient, and/or a third excipient by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of a first excipient, a second excipient, and/or a third excipient by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% of a first excipient, a second excipient, and/or a third excipient by dry weight.

In some embodiments, the unit dosage comprises at least 0.25% stabilizer(s) by dry weight. In some embodiments, the unit dosage comprises at least 0.5% stabilizer(s) by dry weight. In some embodiments, the unit dosage comprises at least 1.6% stabilizer(s) by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more stabilizer(s) by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% stabilizer(s) by dry weight.

In some embodiments, the pharmaceutical composition is a spray-dried pharmaceutical composition. In some embodiments, the pharmaceutical composition comprises a spray-dried composition. In some embodiments, the pharmaceutical composition consults essentially of a spray-dried composition.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a chronic bronchopulmonary disease. A subject can be male or female.

A subject can be one who has been previously diagnosed with or identified as suffering from or having a condition in need of treatment (e.g. a chronic bronchopulmonary disease) or one or more complications related to such a condition, and optionally, have already undergone treatment for a chronic bronchopulmonary disease or the one or more complications related to a chronic bronchopulmonary disease. Alternatively, a subject can also be one who has not been previously diagnosed as having a chronic bronchopulmonary disease or one or more complications related to a chronic bronchopulmonary disease. For example, a subject can be one who exhibits one or more risk factors for a chronic bronchopulmonary disease or one or more complications related to a chronic bronchopulmonary disease or a subject who does not exhibit risk factors.

A “subject in need” of treatment for a particular condition can be a subject having that condition, diagnosed as having that condition, or at risk of developing that condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a chronic bronchopulmonary disease. The term “treating” includes reducing or alleviating at least one adverse effect or symptom of a condition, disease or disorder associated with a chronic bronchopulmonary disease. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable excipient, stabilizer, and/or additive e.g. an excipient, stabilizer, and/or additive commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable excipient, stabilizer, and/or additive can be an excipient, stabilizer, and/or additive other than water. In some embodiments of any of the aspects, a pharmaceutically acceptable excipient, stabilizer, and/or additive can be an artificial or engineered excipient, stabilizer, and/or additive, e.g., an excipient, stabilizer, and/or additive that the active ingredient would not be found to occur in or within nature.

“Pharmaceutically acceptable” excipients, stabilizers, and/or additives are those which can reasonably be administered to a subject to provide an effective dose of the active ingredient employed (e.g., a bacterial preparation). In some embodiments, these are excipients which the Federal Drug Administration (FDA) have to date designated as ‘Generally Regarded as Safe’ (GRAS).

“Powder” means a composition that consists of finely dispersed solid particles that are relatively free flowing and capable of being readily dispersed in an inhalation device and subsequently inhaled by a patient so that the particles are suitable for intranasal or pulmonary administration via the upper respiratory tract.

The “glass transition temperature” is represented by the symbol T_{g} and is the temperature at which a composition changes from a glassy or vitreous state to a syrup or rubbery state. Generally, T_{g} is determined using differential scanning calorimetry (DSC) and is standardly taken as the temperature at which onset of the change of heat capacity (Cp) of the composition occurs upon scanning through the transition. The definition of T_{g} is always arbitrary and there is no present international convention. The T_{g} can be defined as the onset, midpoint or endpoint of the transition. See the article entitled “Formation of Glasses from Liquids and Biopolymers” by C. A. Angell: Science, 267, 1924-1935 (Mar. 31, 1995) and the article entitled “Differential Scanning Calorimetry Analysis of Glass Transitions” by Jan P. Wolanczyk: Cryo-Letters, 10, 73-76 (1989). For detailed mathematical treatment see “Nature of the Glass Transition and the Glassy State” by Gibbs and DiMarzio: Journal of Chemical Physics, 28, NO. 3, 373-383 (March, 1958). These articles are incorporated herein by reference.

A “stable” formulation or composition is one in which the active material therein (e.g., the bacterial preparation) essentially retains its physical stability and/or chemical stability and/or biological activity upon storage. Various analytical techniques for measuring stability are available in the art and are reviewed, e.g., in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. Trend analysis can be used to estimate an expected shelf life before a material has actually been in storage for that time period. In some embodiments, the composition is stable at room temperature (˜25° C.) for at least 3 months, and or stable at about 2-8° C. for at least 1 year. In some embodiments, the composition is stable following freezing (to, e.g., −70° C.) and thawing of the composition.

In some embodiments of any of the aspects, a polypeptide, nucleic acid, cell, or organism as described herein can be engineered. As used herein, “engineered” refers to the aspect of having been manipulated by the hand of man. As is common practice and is understood by those in the art, progeny of an engineered cell is typically still referred to as “engineered” even though the actual manipulation was performed on a prior entity.

In some embodiments of any of the aspects, the bacterial preparations described herein are exogenous to the subject. In some embodiments of any of the aspects, the bacterial preparations described herein are ectopic to the subject. In some embodiments of any of the aspects, the bacterial preparations described herein are not endogenous to the subject.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

In some embodiments of any of the aspects, the bacteria described herein comprise at least one functional heterologous gene. As used herein, the term “heterologous” refers to that which is not endogenous to, or naturally occurring in, a referenced sequence, molecule (including e.g., a protein), virus, cell, tissue, or organism. For example, a heterologous sequence of the present disclosure can be derived from a different species, or from the same species but substantially modified from an original form. Also for example, a nucleic acid sequence that is not normally expressed in a cell or a virus is a heterologous nucleic acid sequence with regard to that cell or virus. The term “heterologous” can refer to DNA, RNA, or protein that does not occur naturally as part of the organism in which it is present or which is found in a location or locations in the genome that differ from that in which it occurs in nature. It is DNA, RNA, or protein that is not endogenous to the virus or cell and has been artificially introduced into the virus or cell.

“Unit dosage” or “unit dose” refers to a receptacle containing a therapeutically effective amount of a pharmaceutical composition as described herein, designed or formulated for administration in a single dose.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an act of inhalation, an act of ingestion, and/or manipulation of a delivery device or machine. Such activity can be performed, e.g., by a medical professional and/or the subject being treated.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A spray-dried biotherapeutic matrix composition comprising a bacterial preparation, wherein the matrix composition is formulated for administration by inhalation.
2. The composition of paragraph 1, wherein the bacterial preparation comprises viable or non-viable bacteria.
3. The composition of paragraph 2, wherein the viable bacteria are capable of actively metabolizing and/or proliferating in the lung of a subject.
4. The composition of paragraph 2, wherein the non-viable bacteria are heat-killed.
5. The composition of paragraph 2, wherein the bacteria are Gram negative.
6. The composition of paragraph 2, wherein the bacteria are Gram positive.
7. The composition of paragraph 2, wherein the bacteria are spore-forming.
8. The composition of paragraph 2, wherein the bacteria are in spore form.
9. The composition of paragraph 2, wherein the bacteria are aerobic.
10. The composition of paragraph 2, wherein the bacteria are anaerobic.
11. The composition of any one of paragraphs 2-10, wherein the bacteria produce at least one immunomodulator.
12. The composition of paragraph 2, wherein the bacteria belong to a genus selected from the group consisting of: Carnobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella.
13. The composition of any one of paragraphs 2-12, wherein the bacterium is Lacticaseibacillus rhamnosus, Lactobacillus acidophilus, or Lactiplantibacillus plantarum.
14. The composition of any one of paragraphs 2-13, wherein the bacteria are non-pathogenic.
15. The composition of any one of paragraphs 2-14, wherein the bacteria are present at a concentration of at least 10¹ colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g.
16. The composition of any one of paragraphs 2-15, wherein the bacteria are present at a concentration of at least 10⁶ colony-forming units per gram (CFU/g).
17. The composition of any one of paragraphs 2-16, wherein the bacteria are present at a concentration of at least 10⁸ colony-forming units per gram (CFU/g).
18. The composition of any one of paragraphs 2-17, wherein the bacteria are resistant to at least one antibiotic.
19. The composition of any one of paragraphs 1-18, wherein the composition comprises at least 0.5% bacterial preparation by dry weight.
20. The composition of paragraph 1, wherein the bacterial preparation comprises a bacterial extract or bacterial metabolite preparation.
21. The composition of paragraph 20, wherein the bacterial extract or bacterial metabolite preparation is selected from the group consisting of: a bacterial exosome; bacterial cell wall; peptidoglycan; teichoic acid; lipoteichoic acid; bacterial S-layer; exopolysaccharide; polysaccharide; a lactic acid polymer; a lactic acid derivative; a lactic acid intermediate; hydrogen peroxide; a bacteriocin; a salivaricin; a reuterin; and a bacterial growth supernatant.
22. The composition of any one of paragraphs 1-21, further comprising at least one excipient.
23. The composition of any one of paragraphs 1-22, further comprising at least two excipients.
24. The composition of paragraph 22 or 23, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; camitine; acetylcamitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.
25. The composition of paragraph 22 or 23, wherein the excipient is leucine and/or trehalose.
26. The composition of any one of paragraphs 1-25, wherein the composition comprises at least 0.5% excipient by dry weight.
27. The composition of any one of paragraphs 1-26, further comprising at least one stabilizer.
28. The composition of paragraph 27, wherein the stabilizer comprises a surfactant.
29. The composition of paragraph 27 or 28, wherein the stabilizer is a polysorbate; poloxamer; or polyvinyl alcohol.
30. The composition of any one of paragraphs 27-29, wherein the stabilizer is Polysorbate 20, Polysorbate 40, Polysorbate 60, or Polysorbate 80.
31. The composition of any one of paragraphs 27-30, wherein the stabilizer is Polysorbate 80.
32. The composition of any one of paragraphs 27-31, wherein the stabilizer is Poloxamer 184, Poloxamer 185, Poloxamer 188, Poloxamer 234, Poloxamer 235, Poloxamer 238, Poloxamer 333, Poloxamer 334, Poloxamer 335, Poloxamer 338, Poloxamer 403, or Poloxamer 407.
33. The composition of any one of paragraphs 1-32, wherein the composition comprises at least 0.25% of the stabilizer or stabilizers by dry weight.
34. The composition of any one of paragraphs 1-33, wherein the composition comprises at least one excipient and at least one stabilizer.
35. The composition of any one of paragraphs 1-34, wherein the composition further comprises at least one additional therapeutic.
36. The composition of paragraph 35, wherein the at least one additional therapeutic is selected from the group consisting of: an anti-inflammatory, an antimicrobial, an antiviral, an antifungal, a vasodilator, and a bronchodilator.
37. The composition of paragraph 36, wherein the at least one additional therapeutic is incorporated into the composition using microencapsulation, co-formulation, or covalent linkage to the composition with a degradable linker.
38. The composition of any one of paragraphs 1-37, wherein the matrix composition comprises a plurality of dried particles.
39. The composition of paragraph 38, wherein the dried particles have a Dv50 of at least 0.5 μm.
40. The composition of paragraph 38, wherein the dried particles have a median mass aerodynamic diameter (MMAD) of at least 1.5 μm to at most 7.5 μm.
41. The composition of any one of paragraphs 38-40, wherein the dried particles have a dispersibility of less than 2.0.
42. The composition of any one of paragraphs 38-41, wherein the dried particles have a dispersibility of at least 0.5 to 1.0.
43. The composition of any one of paragraphs 38-42, wherein the dried particles have a delivered dose of at least 25.0% to at most 125% of the bacterial preparation by mass to a target tissue.
44. The composition of any one of paragraphs 38-43, wherein the dried particles have a delivered dose of at least 60% of the bacterial preparation by mass to a target tissue.
45. The composition of paragraph 44, wherein the target tissue is a target bronchopulmonary tissue.
46. The composition of paragraph 45, wherein the target bronchopulmonary tissue is the lungs, the trachea, the bronchi, the bronchioles, and/or the alveoli.
47. The composition of paragraph 45, wherein the target tissue is a distal tissue site from the lungs delivered via the cardiovascular system or lymphatic system.
48. The composition of any one of paragraphs 38-47, wherein the dried particles have a bulk density of at least 0.1 g/cm³ to 0.8 g/cm³.
49. The composition of any one of paragraphs 38-48, wherein the dried particles have a bulk density of at least 0.5 g/cm³.
50. The composition of any one of paragraphs 38-49, wherein the dried particles have a tapped density of at least 0.1 g/cm³ to 1.0 g/cm³.
51. The composition of any one of paragraphs 38-50, wherein the dried particles have a tapped density of at least 0.6 g/cm³.
52. The composition of any one of paragraphs 38-51, wherein the dried particles have a moisture content of at least 1.0% to 7.0% water by weight by Karl Fischer.
53. The composition of any one of paragraphs 38-52, wherein the dried particles have a moisture content of at least 2.5% water by weight by Karl Fischer.
54. The composition of any one of paragraphs 1-53, wherein the composition is formulated for delivery to the trachea, the bronchi, the bronchioles, and/or the alveoli.
55. The composition of any one of paragraphs 1-54, wherein the composition is formulated for delivery to the lungs.
56. The composition of any one of paragraphs 1-55, wherein the composition is formulated as a capsule.
57. The composition of paragraph 56, wherein the capsule contains at least 10 mg of the spray-dried biotherapeutic matrix composition.
58. The composition of any one of paragraphs 1-57, wherein the composition is formulated for delivery by an inhaler.
59. The composition of any one of paragraphs 1-58, wherein the composition is formulated for delivery by a dry powder inhaler (DPI), a metered dose inhaler (MDI), or a soft-mist inhaler (SMI).
60. The composition of any one of paragraphs 1-59, in combination with an inhaler.
61. An inhalation device for bronchopulmonary delivery comprising:
   a) an inhaler; and
   b) a container containing a spray-dried biotherapeutic matrix composition comprising a bacterial preparation.
62. The device of paragraph 61, wherein the inhaler is a dry powder inhaler (DPI), a metered dose inhaler (MDI), or a soft mist inhaler (SMI).
63. The device of any one of paragraphs 61-62, wherein the inhaler comprises:
   a) a mouthpiece comprising an opening; and
   b) means for aerosolizing or dispersing the spray-dried biotherapeutic matrix composition in the container.
64. A method of preparing a spray-dried biotherapeutic matrix composition comprising a bacterial preparation, comprising:
   a) preparing a liquid feedstock comprising the bacterial preparation;
   b) introducing droplets of the liquid feedstock through an atomization nozzle into a drying chamber;
   c) exposing the liquid feedstock droplets to heated, pressurized gas in the drying chamber to create dried particles; and
   d) isolating dried particles of a predetermined range of diameters in a cyclone chamber, wherein the isolated dried particles comprise the spray-dried biotherapeutic matrix composition.
65. A method of preparing a spray-dried biotherapeutic matrix composition comprising a bacterial preparation, comprising:
   a) obtaining a liquid feedstock comprising the bacterial preparation;
   b) introducing droplets of the liquid feedstock through an atomization nozzle into a drying chamber;
   c) exposing the liquid feedstock droplets to heated, pressurized gas in the drying chamber to create dried particles; and
   d) isolating dried particles of a predetermined range of diameters in a cyclone chamber, wherein the isolated dried particles comprise the spray-dried biotherapeutic matrix composition.
66. The method of paragraph 64 or 65, wherein the step of preparing the liquid feedstock comprises dissolving a solid feedstock into an aqueous solution.
67. The method of any one of paragraphs 64-66, wherein the solid feedstock comprises:
   a) at least 3.5% bacterial preparation by weight;
   b) at least 5% excipient by weight; and/or
   c) at least 0.25% stabilizer by weight.
68. The method of any one of paragraphs 64-67, wherein the solid feedstock comprises:
   a) at least 3.5% bacterial preparation by weight;
   b) at least 5% of a first excipient by weight;
   c) at least 5% of a second excipient by weight; and/or
   d) at least 0.25% stabilizer by weight.
69. The method of any one of paragraphs 64-68, wherein the solid feedstock comprises at least 1% to at most 5% bacterial preparation by weight.
70. The method of any one of paragraphs 64-69, wherein the solid feedstock comprises at least 45% to at most 95% excipient by weight.
71. The method of any one of paragraphs 64-70, wherein the solid feedstock comprises at least 5%-60% of a first excipient by weight, and at least 5%-60% of a second excipient by weight.
72. The method of any one of paragraphs 64-71, wherein the solid feedstock comprises at least 0.25% to at most 10% stabilizer by weight.
73. The method of any one of paragraphs 64-72, wherein the liquid feedstock comprises at least 1 g/L solid feedstock dissolved in an aqueous solution.
74. The method of any one of paragraphs 64-73, wherein the liquid feedstock comprises at least 25 g/L solid feedstock dissolved in an aqueous solution.
75. The method of any one of paragraphs 64-74, wherein the liquid feedstock comprises at least 0.1% to at most 10% solid feedstock dissolved in an aqueous solution.
76. The method of any one of paragraphs 64-75, wherein the liquid feedstock comprises at least 1% solid feedstock dissolved in an aqueous solution.
77. The method of any one of paragraphs 64-76, wherein 1 L of the liquid feedstock comprises:
   a) at least 1.050 g bacterial preparation;
   b) at least 1.5 g excipient;
   c) at least 0.075 g stabilizer; and/or
   d) at least 970 g aqueous solution.
78. The method of any one of paragraphs 64-77, wherein 1 L of the liquid feedstock comprises:
   a) at least 1.050 g bacterial preparation;
   b) at least 0.75 g of a first excipient;
   c) at least 0.75 g of a second excipient;
   d) at least 0.075 g stabilizer; and/or
   e) at least 970 g aqueous solution.
79. The method of any one of paragraphs 64-78, wherein 1 L of the liquid feedstock comprises:
   a) at least 1.050 g bacterial preparation;
   b) at least 0.5 g of a first excipient;
   c) at least 0.5 g of a second excipient;
   d) at least 0.5 g of a third excipient;
   e) at least 0.075 g stabilizer; and/or
   f) at least 970 g aqueous solution.
80. The method of any one of paragraphs 64-79, wherein 1 L of the liquid feedstock comprises at least 750 g to at most 999 g aqueous solution.
81. The method of any one of paragraphs 64-80, wherein the liquid feedstock comprises:
   a) at least 0.105% bacterial preparation;
   b) at least 0.15% excipient;
   c) at least 0.0075% stabilizer; and/or
   d) at least 97% aqueous solution.
82. The method of any one of paragraphs 64-81, wherein the liquid feedstock comprises:
   a) at least 0.105% bacterial preparation by weight;
   b) at least 0.075% of a first excipient by weight;
   c) at least 0.075% of a second excipient by weight;
   d) at least 0.0075% stabilizer and/or
   e) at least 97% aqueous solution by weight.
83. The method of any one of paragraphs 64-82, wherein the liquid feedstock comprises:
   a) at least 0.105% bacterial preparation by weight;
   b) at least 0.05% of a first excipient by weight;
   c) at least 0.05% of a second excipient by weight;
   d) at least 0.05% of a third excipient by weight;
   e) at least 0.0075% stabilizer and/or
   f) at least 97% aqueous solution by weight.
84. The method of any one of paragraphs 64-83, wherein the liquid feedstock comprises at least 0.01% to at most 10% bacterial preparation by weight.
85. The method of any one of paragraphs 64-84, wherein the liquid feedstock comprises at least 1.0% to at most 20% excipient by weight.
86. The method of any one of paragraphs 64-85, wherein the liquid feedstock comprises at least 0.1% to at most 19.8% of a first excipient by weight, and at least 0.1% to at most 19.8% of a second excipient by weight.
87. The method of any one of paragraphs 64-86, wherein the liquid feedstock comprises at least 0.1% to at most 19.8% of a first excipient by weight, at least 0.10% to at most 19.8% of a second excipient by weight, and at least 0.1% to at most 19.8% of a third excipient by weight.
88. The method of any one of paragraphs 64-87, wherein the liquid feedstock comprises at least 0.01% to at most 1.0% stabilizer by weight.
89. The method of any one of paragraphs 64-88, wherein the liquid feedstock comprises at least 75% to at most 99.9% aqueous solution by weight.
90. The method of any one of paragraphs 64-89, wherein the bacterial preparation comprises viable or non-viable bacteria.
91. The method of paragraph 90, wherein the bacteria belong to a genus selected from the group consisting of: Carnobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella.
92. The method of paragraph 90 or 91, wherein the bacterium is Lacticaseibacillus rhamnosus, Lactobacillus acidophilus, or Lactiplantibacillus plantarum.
93. The method of any one of paragraphs 90-92, wherein the bacteria are non-pathogenic.
94. The method of any one of paragraphs 90-93, wherein the bacteria are present at a concentration of at least 101 colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g.
95. The method of any one of paragraphs 90-94, wherein the bacteria are present at a concentration of at least 10⁶ colony-forming units per gram (CFU/g).
96. The method of any one of paragraphs 90-95, wherein the bacteria are present at a concentration of at least 10⁸ colony-forming units per gram (CFU/g).
97. The method of any one of paragraphs 64-96, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; camitine; acetylcamitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.
98. The method of any one of paragraphs 64-97, wherein the excipient is leucine and/or trehalose.
99. The method of any one of paragraphs 64-98, wherein the stabilizer is a polysorbate; poloxamer; or polyvinyl alcohol.
100. The method of any one of paragraphs 64-99, wherein the stabilizer is Polysorbate 80.
101. The method of any one of paragraphs 64-100, wherein the aqueous solution is water.
102. The method of any one of paragraphs 64-101, wherein the liquid feedstock further comprises at least one additional therapeutic.
103. The method of paragraph 102, wherein the at least one additional therapeutic is selected from the group consisting of: an anti-inflammatory, an antimicrobial, an antiviral, an antifungal, a vasodilator, and a bronchodilator.
104. The method of any one of paragraphs 64-103, wherein the atomization nozzle into the drying chamber has a diameter of at least 1.2 um.
105. The method of any one of paragraphs 64-104, wherein the droplets of liquid feedstock produced by the atomization nozzle into the drying chamber have a diameter of at least 1.5 um.
106. The method of any one of paragraphs 64-105, wherein the droplets of liquid feedstock have a flow rate through the drying chamber of at least 0.5 g/min.
107. The method of any one of paragraphs 64-106, wherein the droplets of liquid feedstock have a flow rate through the drying chamber of at least 15 g/min.
108. The method of any one of paragraphs 64-107, the droplets of liquid feedstock have a flow rate through the drying chamber of at most 1000 g/min.
109. The method of any one of paragraphs 64-108, wherein the heated, pressurized gas is heated before being inlet into the drying chamber.
110. The method of any one of paragraphs 64-109, wherein the heated, pressurized gas is inlet into the drying chamber at a temperature of at least 100° C.
111. The method of any one of paragraphs 64-110, wherein the heated, pressurized gas is inlet into the drying chamber at a temperature of at most 195° C.
112. The method of any one of paragraphs 64-111, wherein the heated, pressurized gas is outlet from the drying chamber at a temperature of at least 40° C.
113. The method of any one of paragraphs 64-112, wherein the heated, pressurized gas is outlet from the drying chamber at a temperature of at least 48° C.
114. The method of any one of paragraphs 64-113, wherein the heated, pressurized gas is outlet from the drying chamber at a temperature of at most 85° C.
115. The method of any one of paragraphs 64-114, wherein the heated, pressurized gas is pressurized before being inlet into the drying chamber.
116. The method of any one of paragraphs 64-115, wherein the heated, pressurized gas in the drying chamber has an atomization gas pressure of at least 10 pounds per square inch gauge (psig).
117. The method of any one of paragraphs 64-116, wherein the heated, pressurized gas in the drying chamber has an atomization gas pressure of at least 20 pounds per square inch gauge (psig).
118. The method of any one of paragraphs 64-117, wherein the heated, pressurized gas in the drying chamber has an atomization gas pressure of at most 150 pounds per square inch gauge (psig).
119. The method of any one of paragraphs 64-118, wherein the heated, pressurized gas has a flow rate through the drying chamber of at least 5 kg/hr.
120. The method of any one of paragraphs 64-119, wherein the heated, pressurized gas has a flow rate through the drying chamber of at least 18 kg/hr.
121. The method of any one of paragraphs 64-120, wherein the heated, pressurized gas has a flow rate through the drying chamber of at most 150 kg/hr.
122. The method of any one of paragraphs 64-121, wherein the heated, pressurized gas is outlet through the cyclone chamber.
123. The method of any one of paragraphs 64-122, wherein the step of exposing the liquid feedstock droplets to heated, pressurized gas in the drying chamber takes at most 8 hours.
124. The method of any one of paragraphs 64-123, wherein the dried particles isolated in the cyclone chamber have a median mass aerodynamic diameter (MMAD) of at least 1.5 μm to at most 7.5 μm.
125. The method of any one of paragraphs 64-124, wherein the dried particles isolated in the cyclone chamber have a median mass aerodynamic diameter (MMAD) of at least 4.0 μm.
126. The method of any one of paragraphs 64-125, wherein the step of isolating dried particles of a predetermined range of diameters in the cyclone chamber occurs continuously.
127. The method of any one of paragraphs 64-126, wherein the spray-dried biotherapeutic matrix composition comprises a bacterial viability of at least 5% after the step of isolating the dried particles.
128. A method of delivering a spray-dried biotherapeutic matrix composition comprising a bacterial preparation to a subject, comprising:
   a) obtaining an inhalation device for bronchopulmonary delivery comprising:
      i) an inhaler; and
      ii) a container containing a spray-dried biotherapeutic matrix composition comprising a bacterial preparation;
   b) activating the inhaler to cause aerosolization or dispersal of the spray-dried biotherapeutic matrix composition; and
   c) inhaling the aerosolized or dispersed spray-dried biotherapeutic matrix composition.
129. A method of delivering a spray-dried biotherapeutic matrix composition comprising a bacterial preparation to a subject, comprising:
   a) obtaining an inhalation device for bronchopulmonary delivery comprising:
      i) an inhaler; and
      ii) a container containing the spray-dried biotherapeutic matrix composition of any one of paragraphs 1-60;
   b) activating the inhaler to cause aerosolization or dispersal of the spray-dried biotherapeutic matrix composition; and
   c) inhaling the aerosolized or dispersed spray-dried biotherapeutic matrix composition.
130. A method of delivering a spray-dried biotherapeutic matrix composition comprising a bacterial preparation to a subject, comprising:
   a) obtaining the inhalation device of any one of paragraphs 61-63;
   b) activating the inhaler to cause aerosolization or dispersal of the spray-dried biotherapeutic matrix composition; and
   c) inhaling the aerosolized or dispersed spray-dried biotherapeutic matrix composition.
131. The method of any one of paragraphs 128-130, wherein the inhaler is a dry powder inhaler (DPI), a metered dose inhaler (MDI), or a soft mist inhaler (SMI).
132. The method of any one of paragraphs 128-131, wherein the inhaler comprises:
   a) a mouthpiece comprising an opening; and
   b) means for aerosolizing or dispersing the spray-dried biotherapeutic matrix composition in the container.
133. The method of any one of paragraphs 128-132, wherein the inhaler has an inspiration flow rate of at least 15 L/min.
134. The method of any one of paragraphs 128-133, wherein at least 25% to at most 100% of the spray-dried biotherapeutic matrix composition by mass is delivered to a target bronchopulmonary tissue.
135. The method of any one of paragraphs 128-134, wherein at least 60.0% of the spray-dried biotherapeutic matrix composition by mass is delivered to a target bronchopulmonary tissue.
136. The method of any one of paragraphs 128-135, wherein the target bronchopulmonary tissue is the lungs, the trachea, the bronchi, the bronchioles, and/or the alveoli.
137. The method of any one of paragraphs 128-136, wherein the spray-dried biotherapeutic matrix composition is delivered from the bronchopulmonary tissue to a distal tissue site via the cardiovascular system or lymphatic system.
138. A method of treating a subject in need thereof comprising administering through inhalation an effective dose of a spray-dried biotherapeutic matrix composition comprising a bacterial preparation.
139. A method of treating a subject in need thereof comprising administering through inhalation an effective dose of the spray-dried biotherapeutic matrix composition of any one of paragraphs 1-60.
140. The method of paragraph 138 or 139, wherein the subject has been diagnosed with or is at risk of developing a chronic bronchopulmonary disease.
141. The method of any one of paragraphs 138-140, wherein the chronic bronchopulmonary disease is selected from the group consisting of: chronic obstructive pulmonary disease (COPD), lung cancer, asthma, bronchiectasis, emphysema, cystic fibrosis (CF), bronchopulmonary dysplasia (BPD), acute respiratory disease syndrome (ARDS), idiopathic pulmonary fibrosis (IPF), pulmonary hypertension (PAH), silicosis, interstitial lung disease (ILD), and pleural effusion (PE).
142. The method of any one of paragraphs 138-141, wherein the lung cancer is small cell lung cancer (SCLC) or non-small cell lung cancer (NSCLC).
143. The method of any one of paragraphs 138-142, wherein the subject has been diagnosed with or is at risk of contracting an infectious pulmonary disease.
144. The method of any one of paragraphs 138-143, wherein the infectious bronchopulmonary disease is caused by or associated with an infectious agent selected from: adenovirus; coronavirus; influenza virus; parainfluenza virus; parvovirus; respiratory syncytial virus; rhinovirus; enterovirus; measles virus; rubella virus; varicella virus; Corynebacterium diphtheriae; Haemophilus influenzae; Legionella pneumophila; Bordetella pertussis; Mycobacterium tuberculosis; Streptococcus species; Pseudomonas species; Escherichia coli; Aspergillus species; Cryptococcus species; and Pneumocystis species.
145. The method of any one of paragraphs 138-144, wherein the spray-dried biotherapeutic matrix composition is administered in conjunction with a standard of care for the chronic or infectious bronchopulmonary disease.
146. The method of any one of paragraphs 138-145, wherein the spray-dried biotherapeutic matrix composition is administered using an inhaler.
147. The method of any one of paragraphs 138-146, wherein the inhaler is a dry powder inhaler (DPI), a metered dose inhaler (MDI), or a soft mist inhaler (SMI).
148. The method of any one of paragraphs 138-147, wherein the effective dose of the spray-dried biotherapeutic matrix composition is at least 10⁴ CFU of bacteria per unit dose.
149. The method of any one of paragraphs 138-148, wherein the effective dose of the spray-dried biotherapeutic matrix composition is at least 10⁴ CFU of viable bacteria per unit dose.
150. The method of any one of paragraphs 138-149, wherein the spray-dried biotherapeutic matrix composition reduces neutrophilic inflammation in a target tissue.
151. The method of any one of paragraphs 138-150, wherein the spray-dried biotherapeutic matrix composition increases lactic acid concentration in a target tissue by at least 25%.
152. The method of any one of paragraphs 138-151, further comprising administering at least one additional therapeutic.
153. The method of paragraph 152, wherein the at least one additional therapeutic is selected from the group consisting of: an anti-inflammatory, an antimicrobial, an antiviral, an antifungal, a vasodilator, and a bronchodilator.
154. The method of paragraph 152, wherein the spray-dried biotherapeutic matrix composition comprises the bacterial preparation and the at least one additional therapeutic.
155. The method of paragraph 152, wherein the spray-dried biotherapeutic matrix composition is co-administered with the at least one additional therapeutic.
156. The method of paragraph 152, wherein the co-administration comprises administering using a combination delivery device.
157. A unit dosage form comprising at least 1 mg of a spray-dried biotherapeutic matrix composition comprising a bacterial preparation.
158. A unit dosage form comprising at least 1 mg of the spray-dried biotherapeutic matrix composition of any one of paragraphs 1-60.
159. A unit dosage form comprising at least 1 mg of the spray-dried biotherapeutic matrix composition prepared by the methods of any one of paragraphs 64-127.
160. A unit dosage form comprising at least 1 mg of a spray-dried biotherapeutic matrix composition comprising at least 10⁴ CFU bacteria per unit dose.
161. The unit dosage form of any one of paragraphs 157-160, wherein the dosage is at least 30 mg spray-dried biotherapeutic matrix composition.
162. The unit dosage form of any one of paragraphs 157-161, wherein the dosage comprises at least 10⁴ CFU of bacteria per unit dose.
163. The unit dosage form of any one of paragraphs 157-162, wherein the dosage comprises at least 10⁴ CFU of viable bacteria per unit dose.

The technology described herein is further illustrated by the following examples which in no way should be construed as being further limiting.

### EXAMPLES

### Example 1

FIG. 1 shows an exemplary spray drying schematic.

As shown in FIG. 2, a Dry Powder Inhaler (DPI), such as PLASTIAPE RS01 DPI, can be used for delivery of inhaled formulations, as described herein.

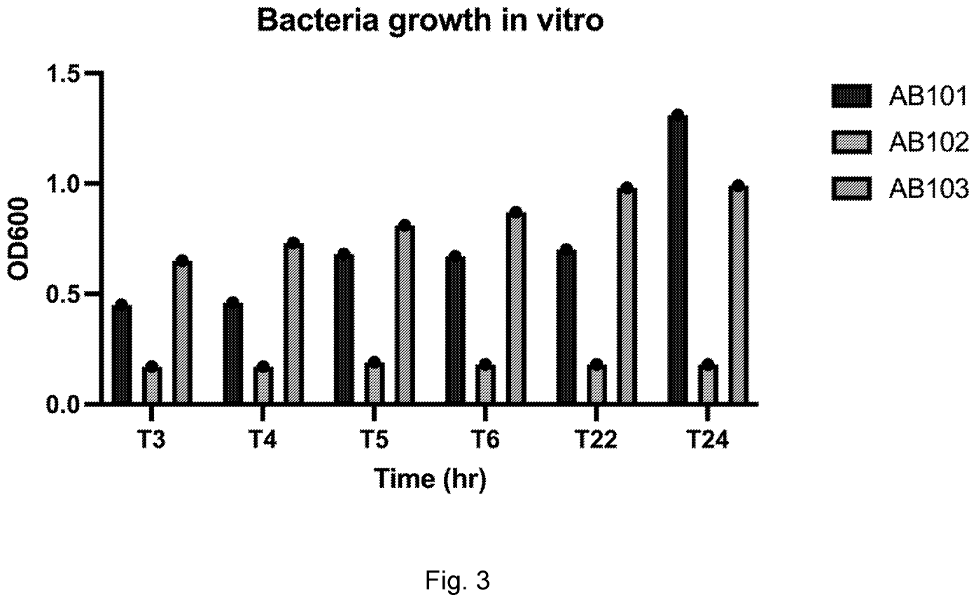

In FIG. 3, three bacterial strains AB101, AB102, and AB103 were assessed for their growth and viability in vitro over the course of 24 hours. 5×10⁷ cells/mL of bacteria for each strain was cultured in MRS broth at 37° C. in a shaker at 200 revolutions per minute (RPM) for 16 hours. OD600 was measured for each strain at 3, 4, 5, 6, 22, and 24 hours.

In FIG. 4A-4C healthy mice were inoculated intratracheally with a blend of live bacterial strains AB101, 102, and 103 in a 1:1:1 ratio or a negative control. Bacterial load in the lung tissue and lactic acid output in the bronchoalveolar lavage (BAL) fluid of the mice was measured at 0-hours, 4-hours, 8-hours, 12-hours, 16-hours, 24-hours, and 72-hours post-inoculation.

In FIG. 5A-5B, spray dried powder was enumerated for viability. Three batches were tested in two types of media. F4 solution contained 0.05% Polysorbate 80 in water for injection, and F6 media contained 5:55 MRS:F4. Liquid feedstock solution was the solution pre-drying. The final spray dried powder was diluted 1:10⁸, and 50 μL of each solution was plated on agarose plates. Plates were incubated for 16 hours at 37° C., colonies counted, and total CFU calculated.

In FIG. 6, particle characteristics were determined for two exemplary formulations of spray dried live biotherapeutic product AB1000 using the indicated test method.

In FIG. 7, the viability of the bacteria in the spray-dried powder for each batch was measured by International Organization for Standardization (ISO) 7889 enumeration standard.

In FIG. 8, the resistance and susceptibility profiles of live bacterial strains AB101, AB102, and AB103 to 51 antibiotics was measured.

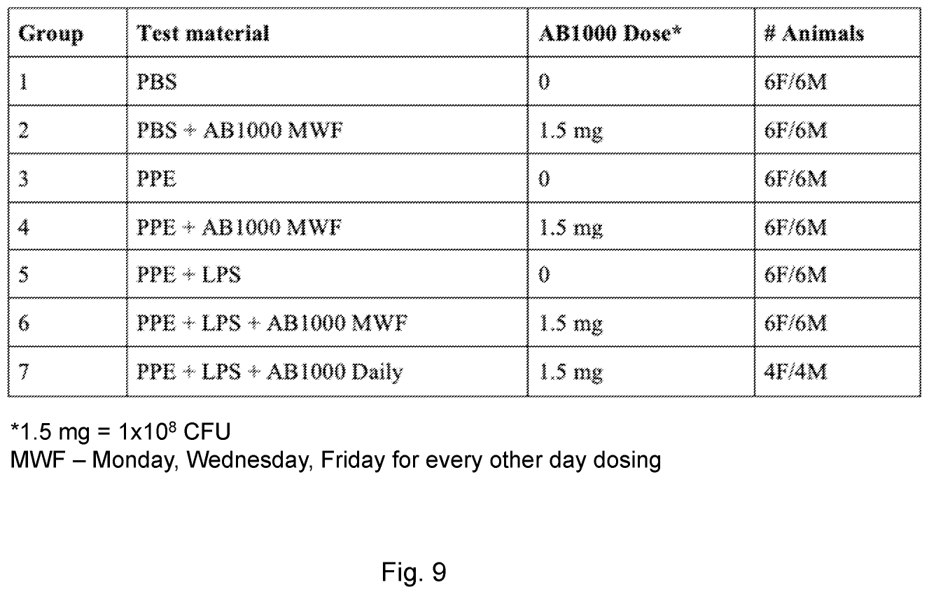

FIG. 9 shows the group allocation for testing mice exposed to porcine pancreatic elastase (PPE) with or without lipopolysaccharide (LPS) dosed with representative drug powder of live biotherapeutic AB1000. This testing scheme is referred to herein as “PPE model mice.”

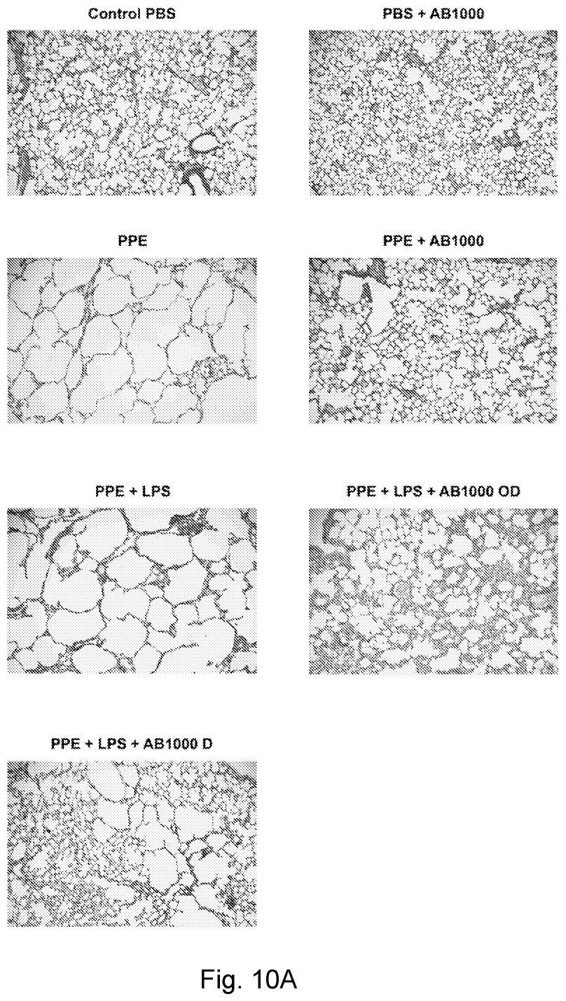

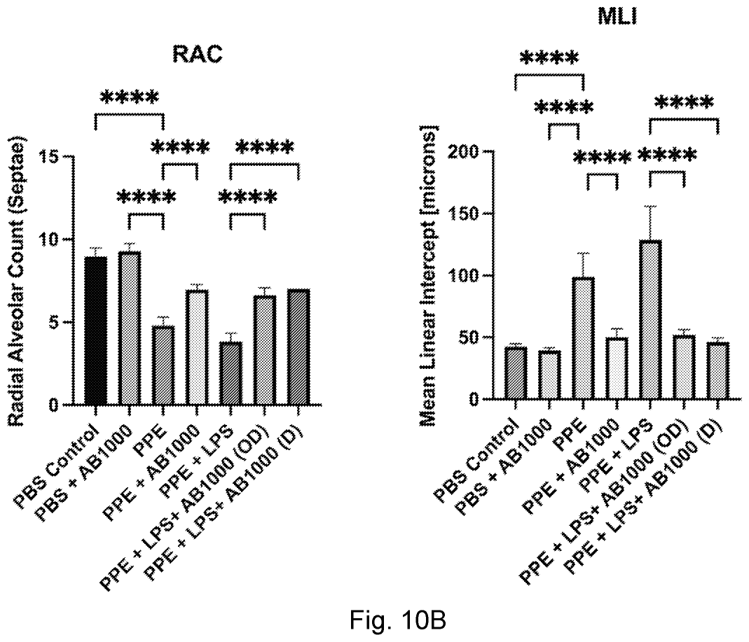

In FIG. 10A-10B, changes in lung structure of mice groups in the PPE model was measured, including radial alveolar count (RAC) and mean linear intercept (MLI) of mouse lung tissue.

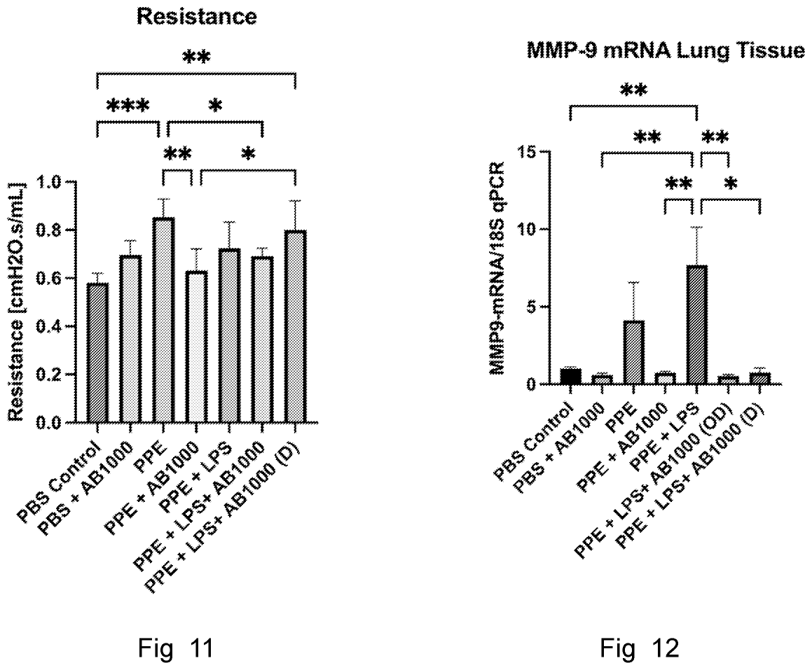

In FIG. 11, pulmonary function testing was measured using lung resistance in the PPE model mice.

In FIG. 12, mRNA transcription levels of MMP-9 were measured in lung tissue of the PPE model mice.

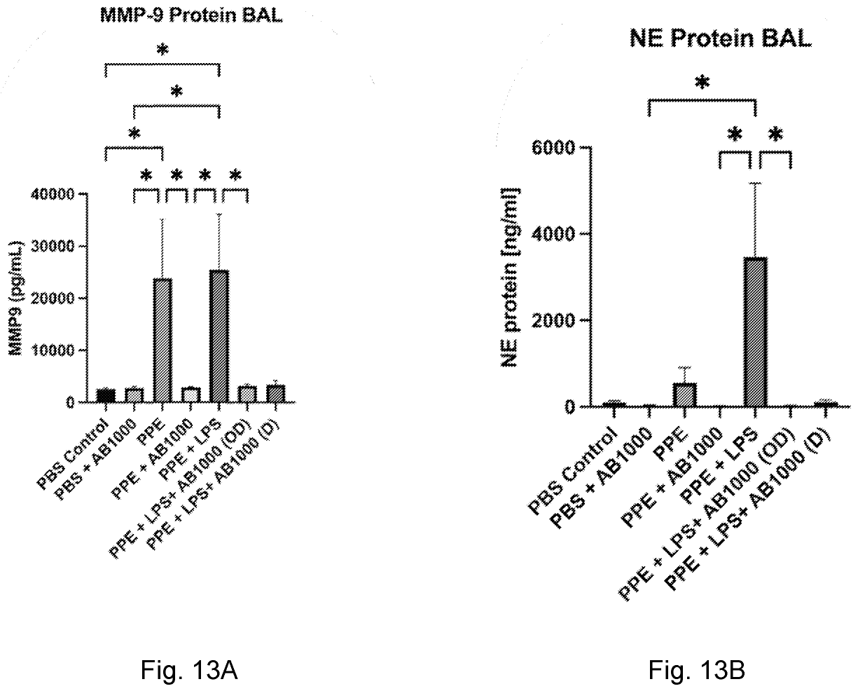

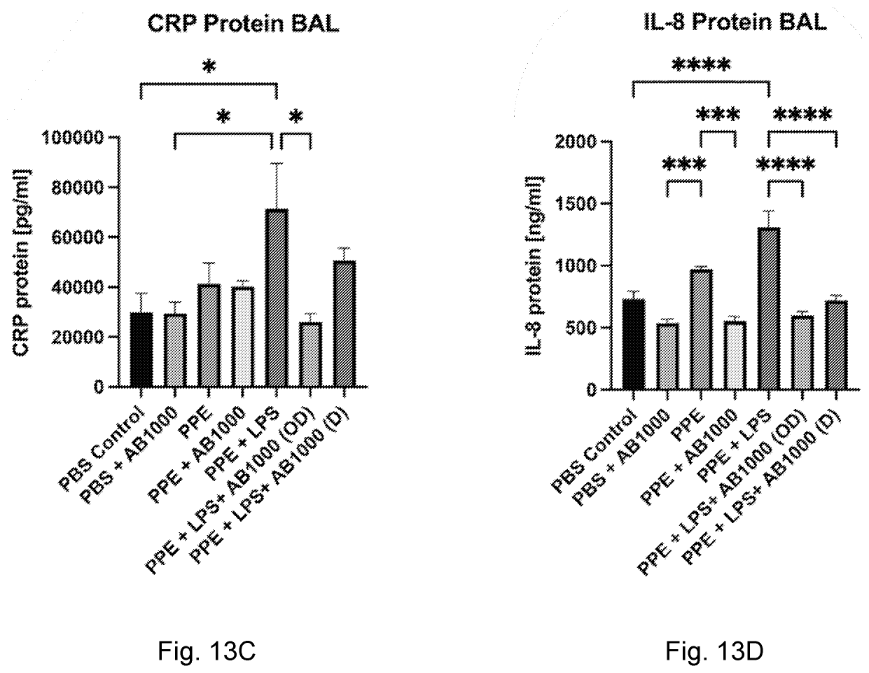

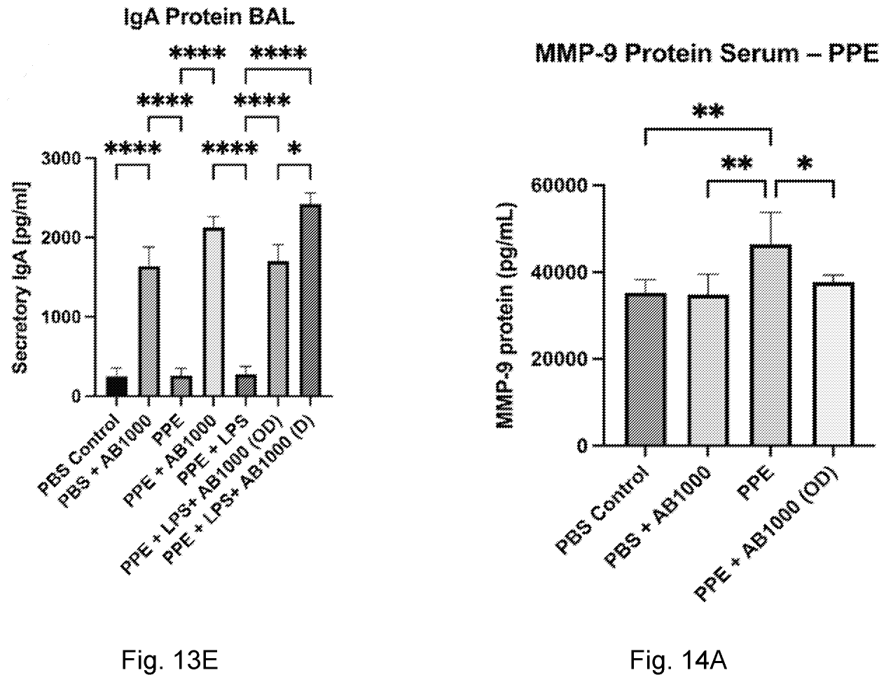

In FIG. 13A-13E, the protein levels of markers of inflammation were measured in the bronchoalveolar lavage (BAL) fluid of the PPE model mice.

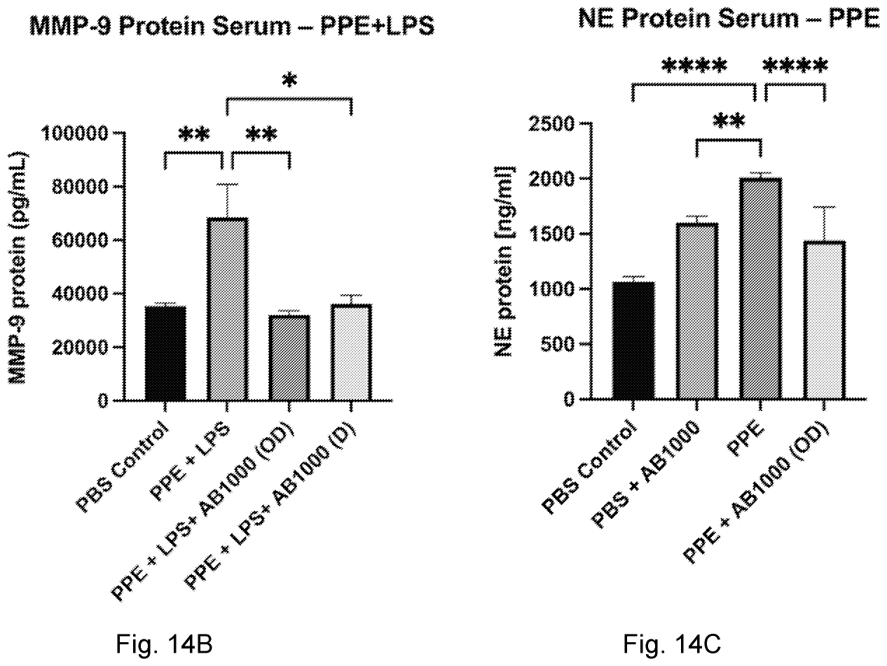

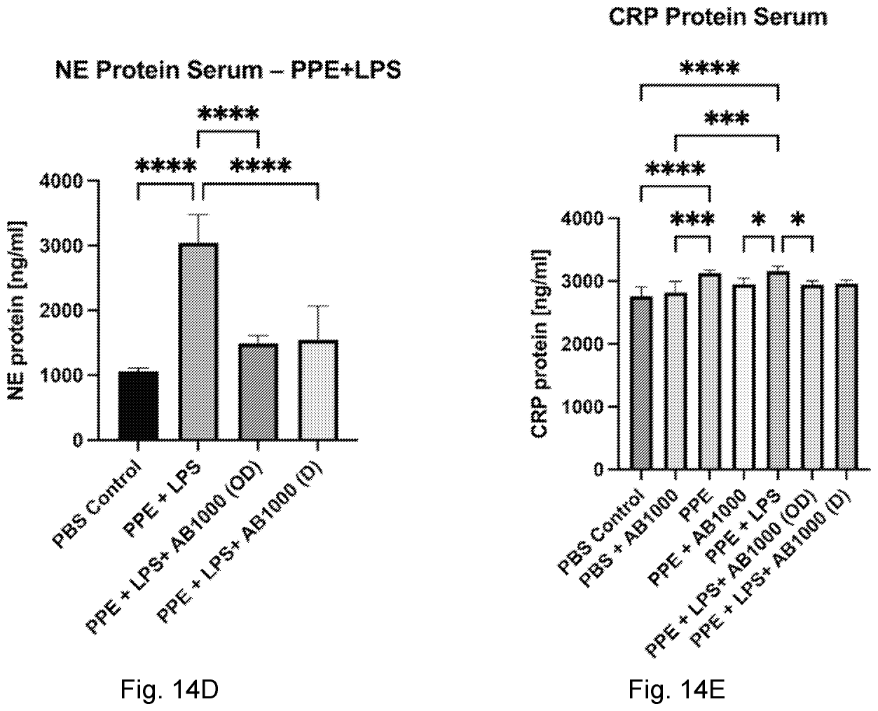

In FIG. 14A-14E, the protein levels of markers of inflammation were measured in the serum of the PPE model mice.

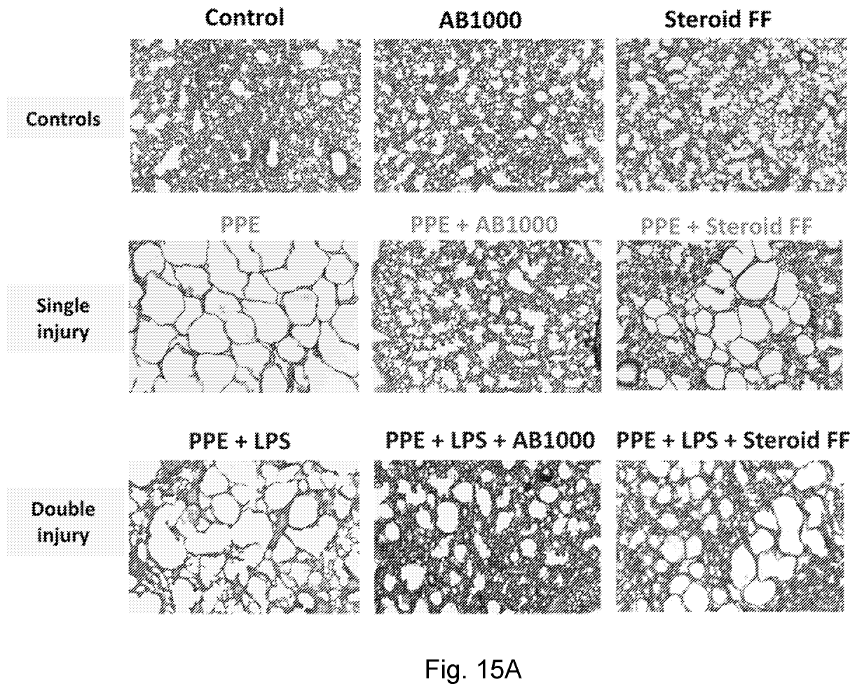

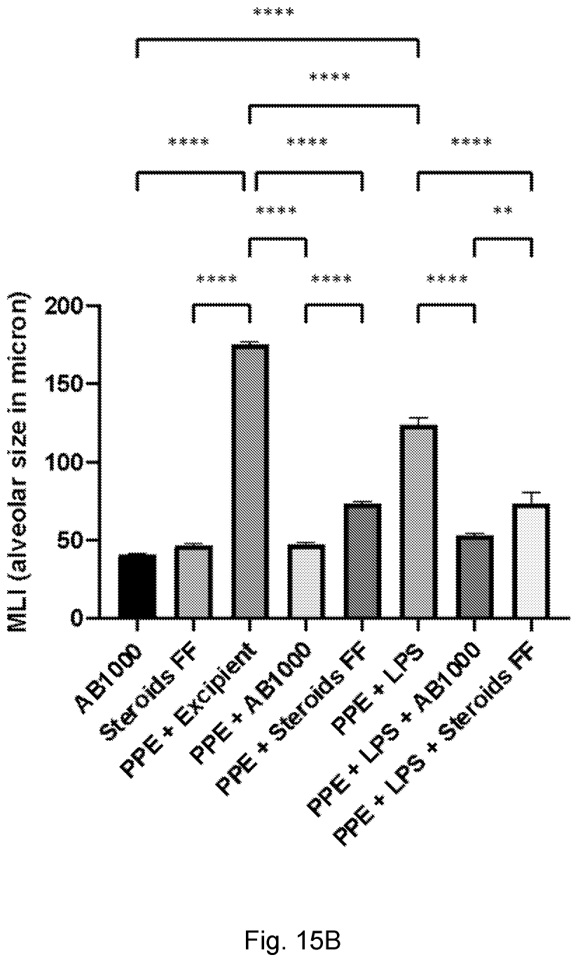

In FIG. 15A-15B, lung tissue structure, including mean linear intercept (MLI), was measured in mice exposed to PPE and LPS and those treated with AB1000 or fluticasone furoate, an inhaled steroid.

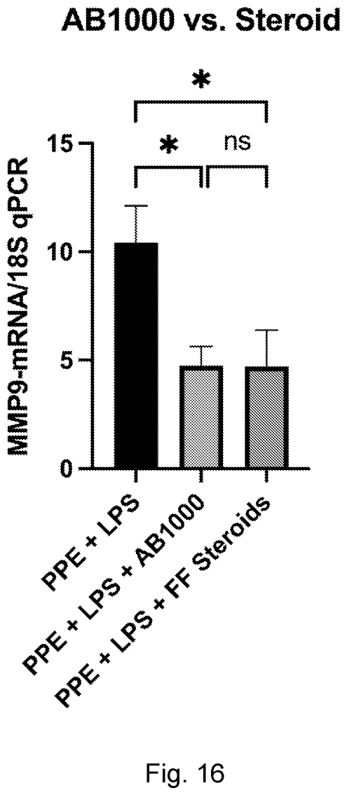

In FIG. 16, the effect of AB1000 and fluticasone furoate on reducing MMP-9 expression in lung tissue was measured in mice exposed to PPE and LPS.

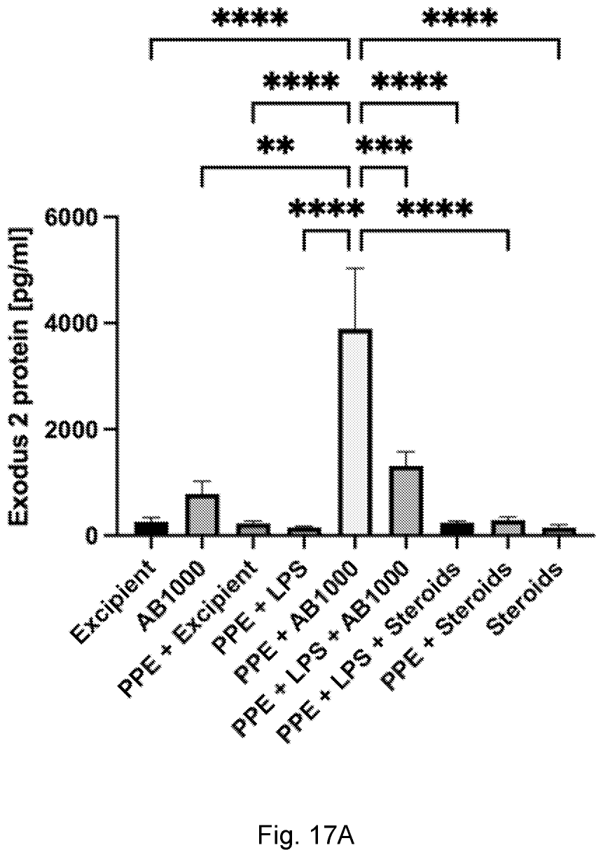

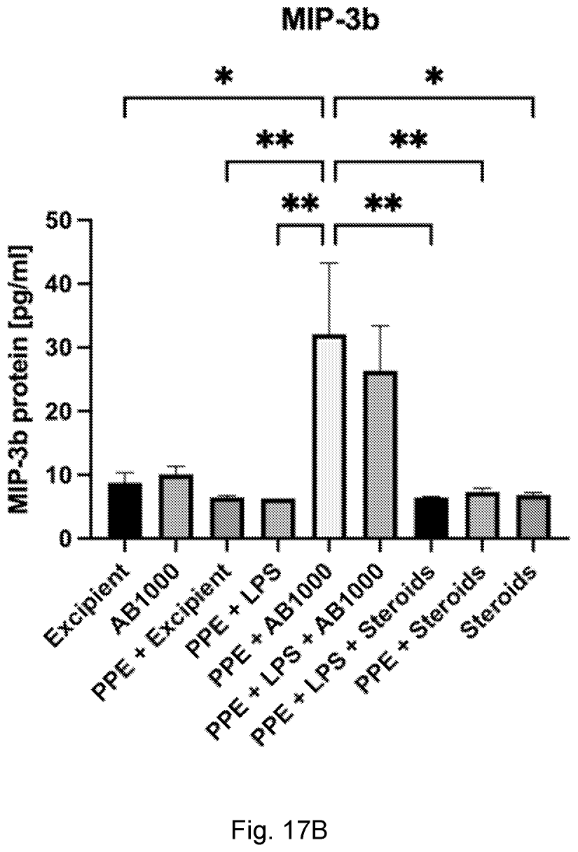

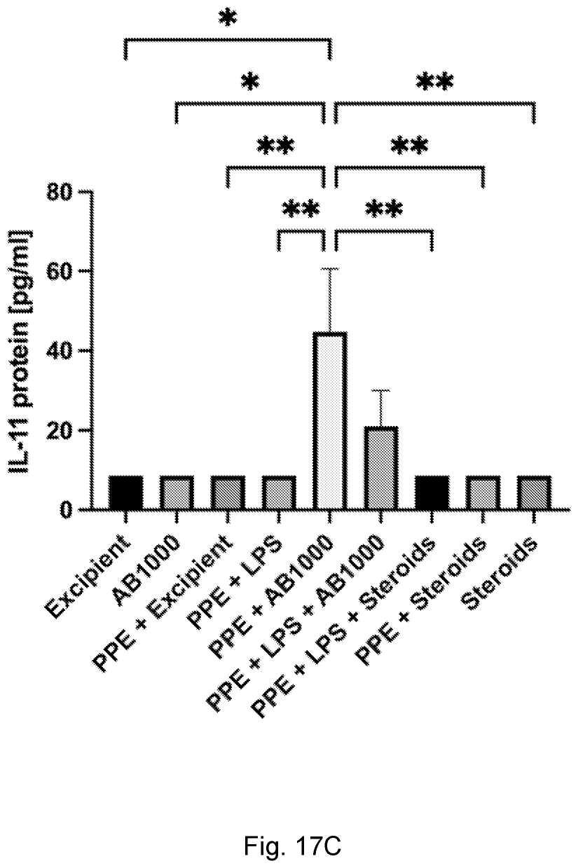

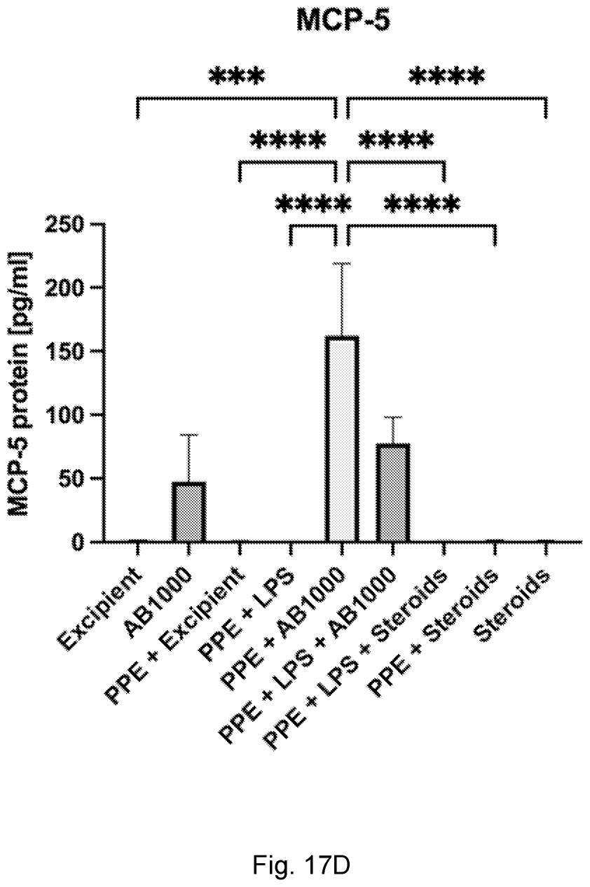

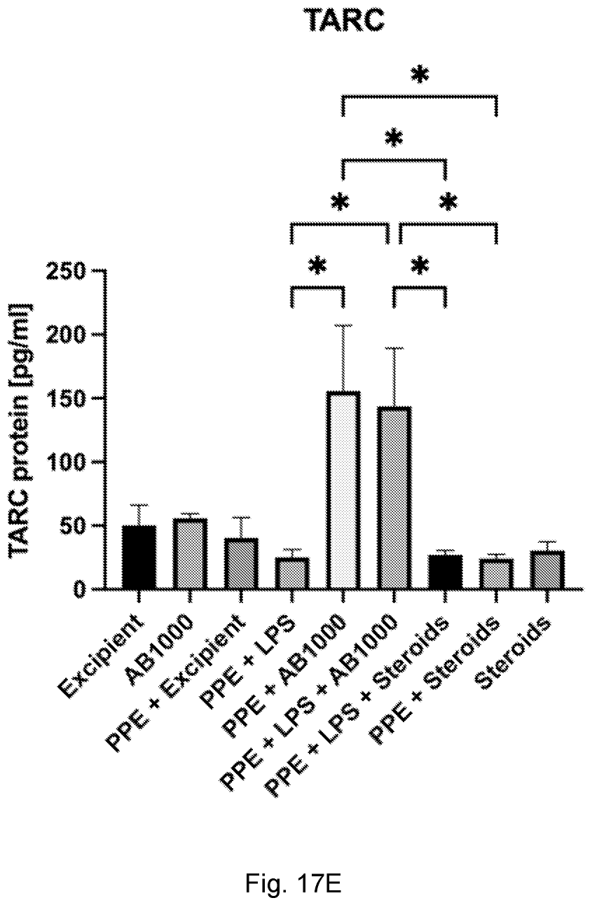

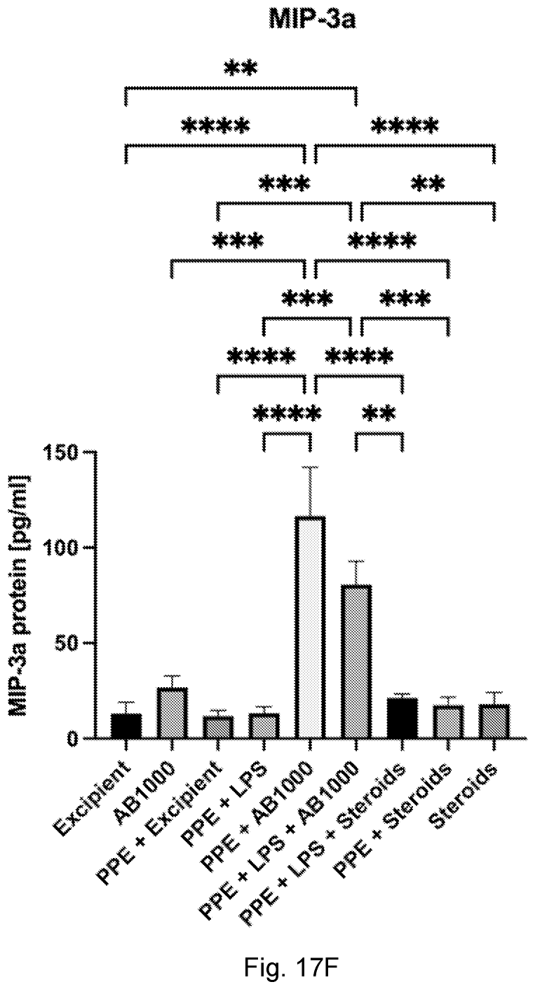

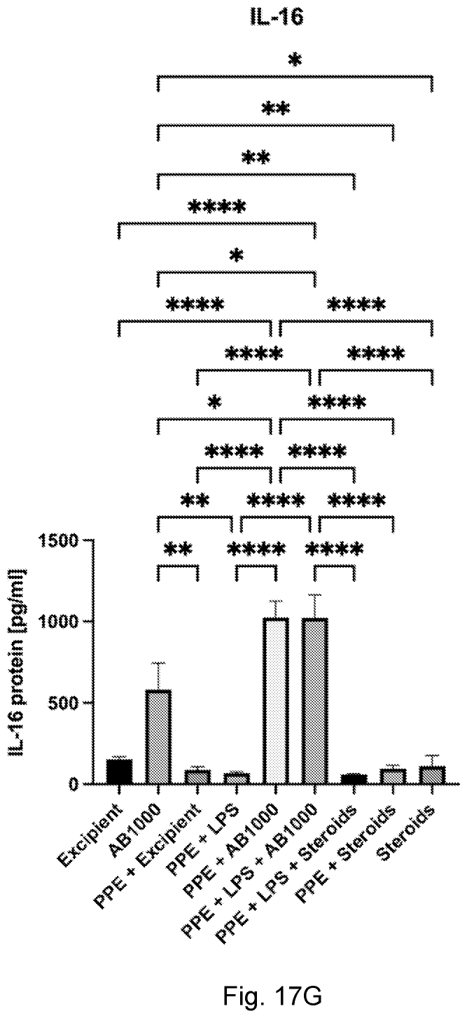

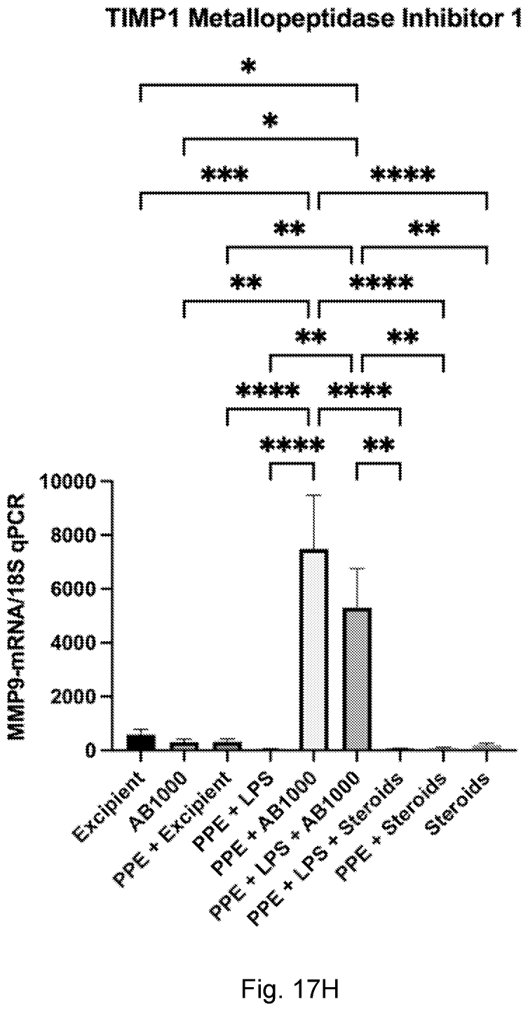

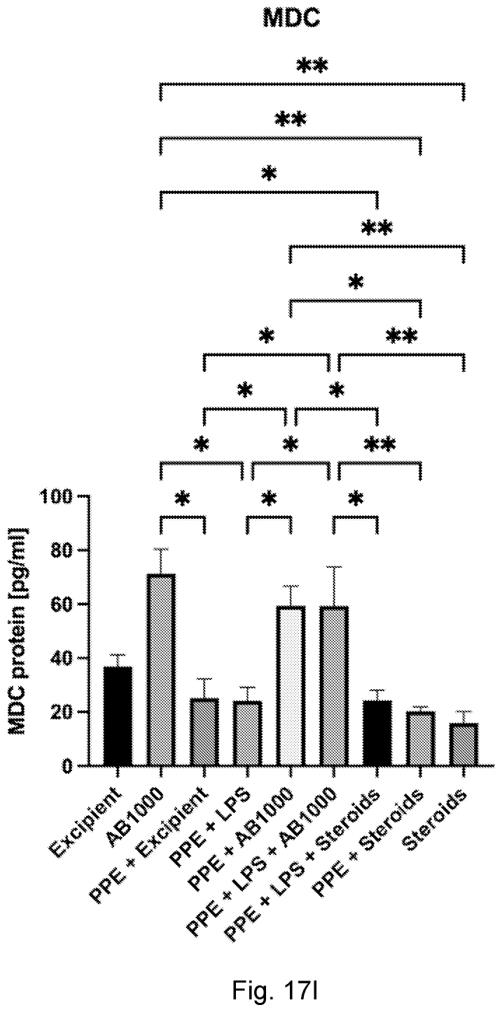

In FIG. 17A-17I, protein levels of anti-inflammatory cytokines Exodus 2 (FIG. 17A), Macrophage Inflammatory protein-3b (MIP-3b) (FIG. 17B), interleukin-11 (IL-11) (FIG. 17C), monocyte chemotactic protein 5 (MCP-5) (FIG. 17D), thymus- and activation-regulated chemokine (TARC) (FIG. 17E), MIP-3a (FIG. 17F), IL-16 (FIG. 17G), tissue inhibitor of metalloproteinases 1 (TIMP1) (FIG. 17G), and macrophage-derived chemokine (MDC) (FIG. 17I) were measured in the bronchoalveolar lavage fluid (BAL) of mice exposed to PPE+LPS which were treated with AB1000.

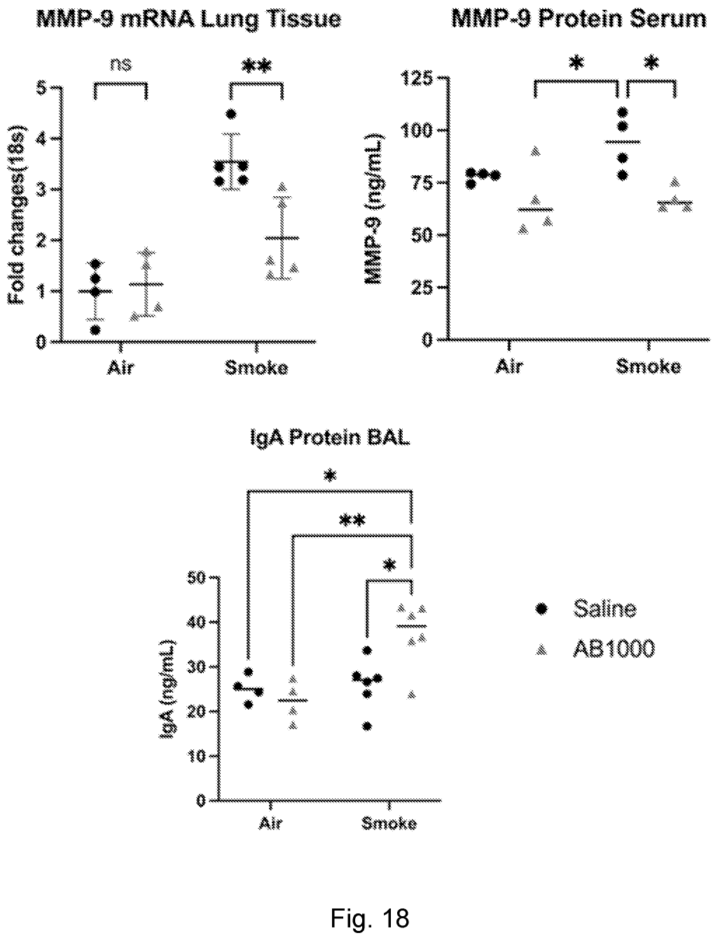

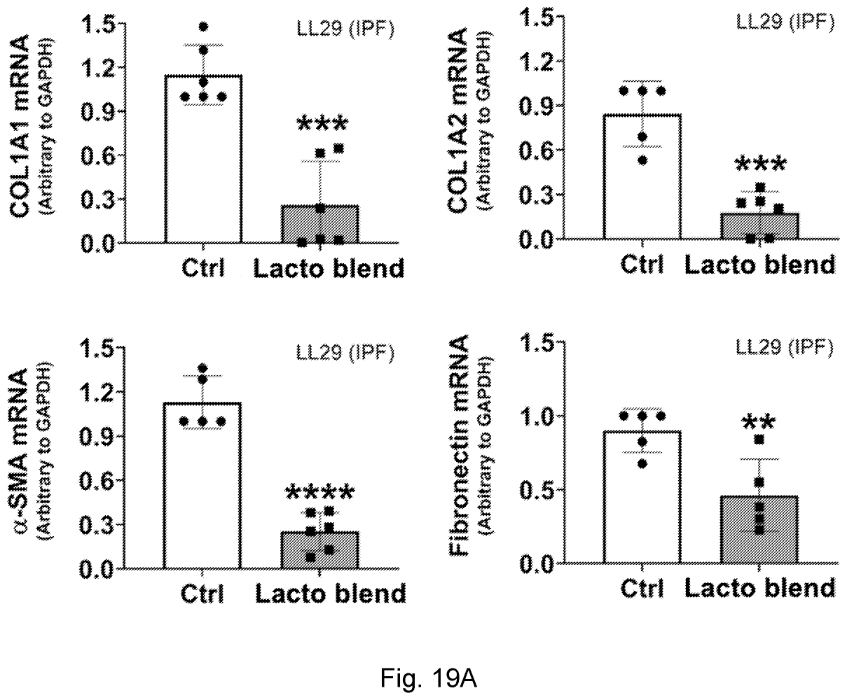

In FIG. 18, MMP-9 expression in lung tissue, MMP-9 protein in serum, and IgA protein in the BAL were measured in mice exposed to cigarette smoke or control air treated with AB1000 or control saline treatment.

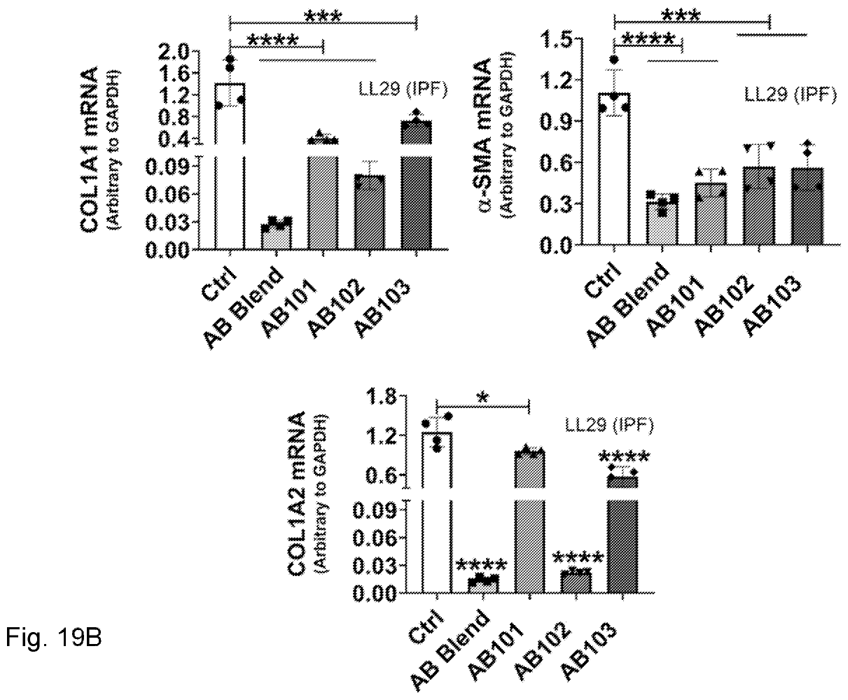

In FIG. 19A-19B, markers of fibrogenesis (smooth muscle alpha-actin (αSMA)) and fibrosis development (collagen type I alpha 1 (COL1A1), COL1A2, fibronectin) were measured in vitro in LL29 idiopathic pulmonary fibrosis (IPF) fibroblasts treated with Lacto blend.

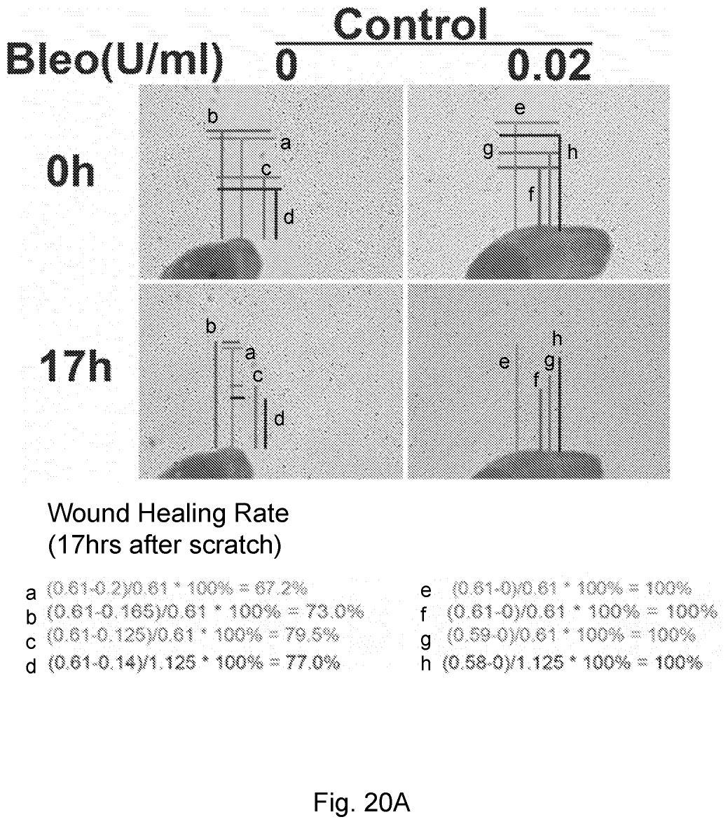

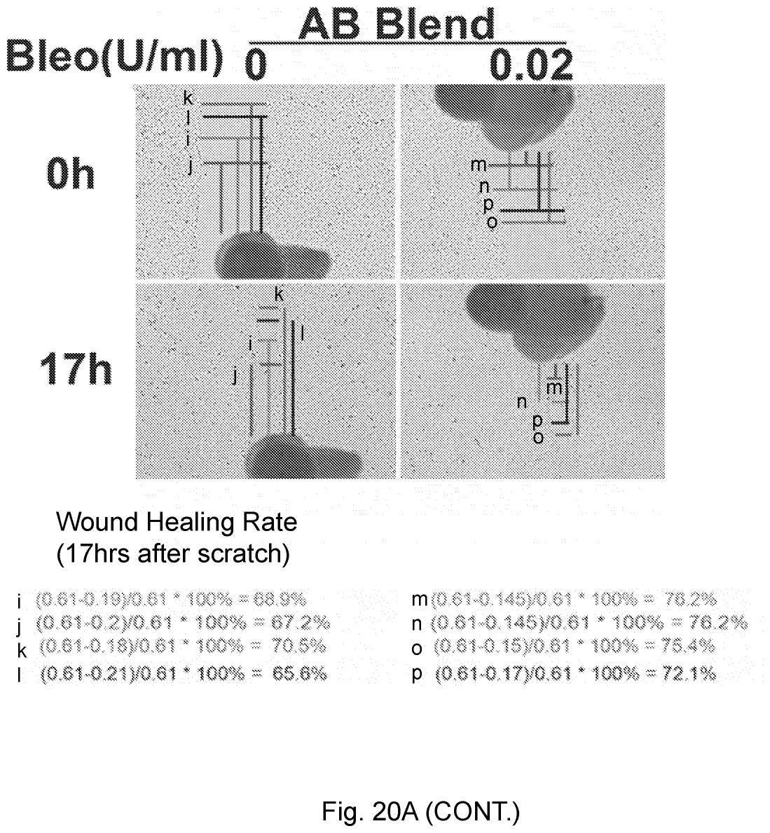

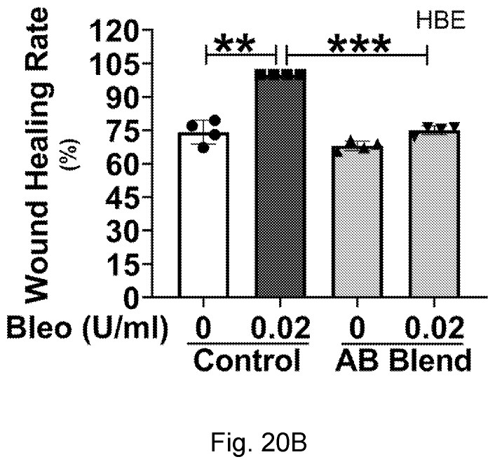

In FIG. 20A-20B, the epithelial to mesenchymal transition was measured in bleomycin-exposed human bronchial epithelial cells treated with a negative control or AB blend.

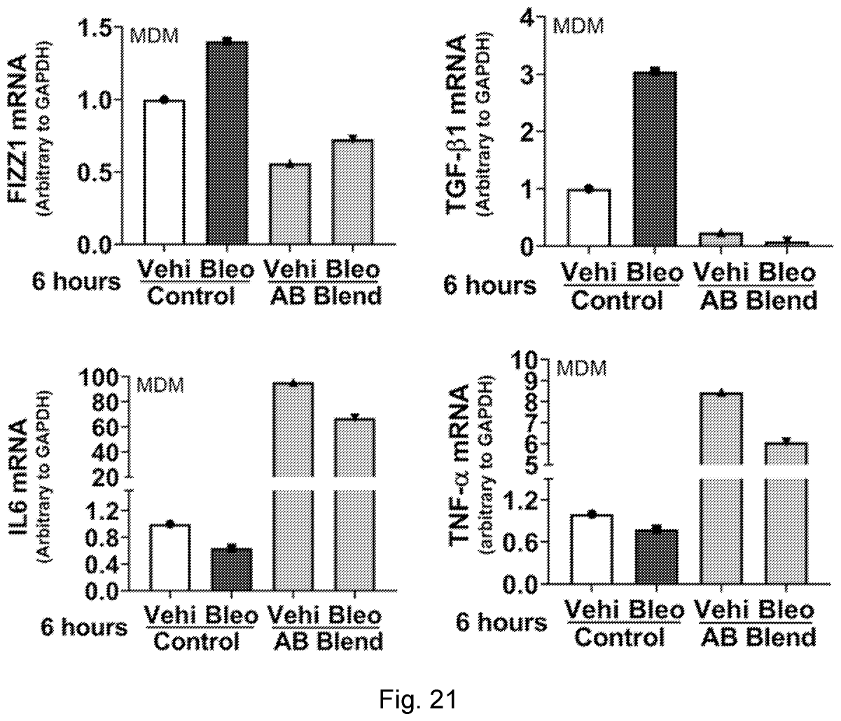

In FIG. 21, profibrotic markers (transforming growth factor-beta-1 (TGFbeta-1), found in inflammatory zone protein 1 (FIZZ1; also referred to as Resistin-like molecule alpha) and anti-fibrotic markers (IL-6, tumour necrosis factor alpha (TNF-alpha)) were measured in in THP1 monocytes (a human leukemia monocytic cell line) exposed to bleomycin and treated with a negative control or AB blend.

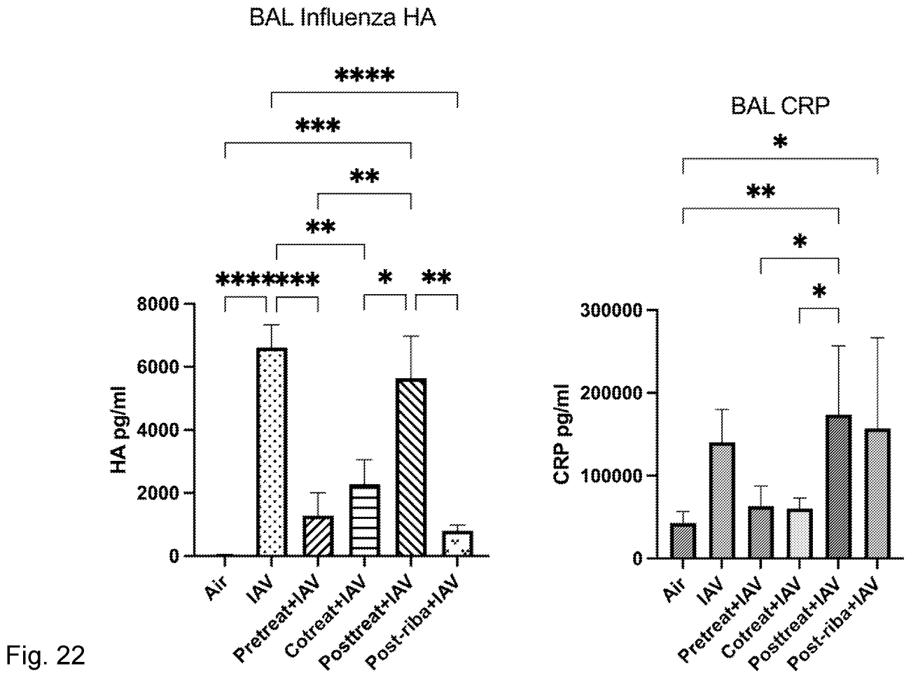

In FIG. 22, influenza A (IAV) virus hemagglutinin (HA) protein and C-reactive protein (CRP) were measured in BAL of IAV-infected mice treated with negative control or Lacto blend administered prior to IAV infection (pretreat+IAV), at the same time as IAV infection (cotreat+IAV), or after IAV infection (posttreat+IAV), or as a positive control ribavirin administered after IAV infection (post-riba+IAV). Pretreat+IAV: Lacto blend inhaled for 5 min (5 mg/day) on 6 consecutive days, then infected with AIV. Co-treat+IAV: Lacto blend inhaled for 5 min one time and immediately infected with IAV. Post-treat+IAV: IAV infection, 24 hours later give first dose of Lacto blend (5 min, 5 mg/day for 6 days). Post-ribavirin+IAV: IAV infection, 24 hours later give 100 mg/kg/day of ribavirin intraperitoneally for 4 days.

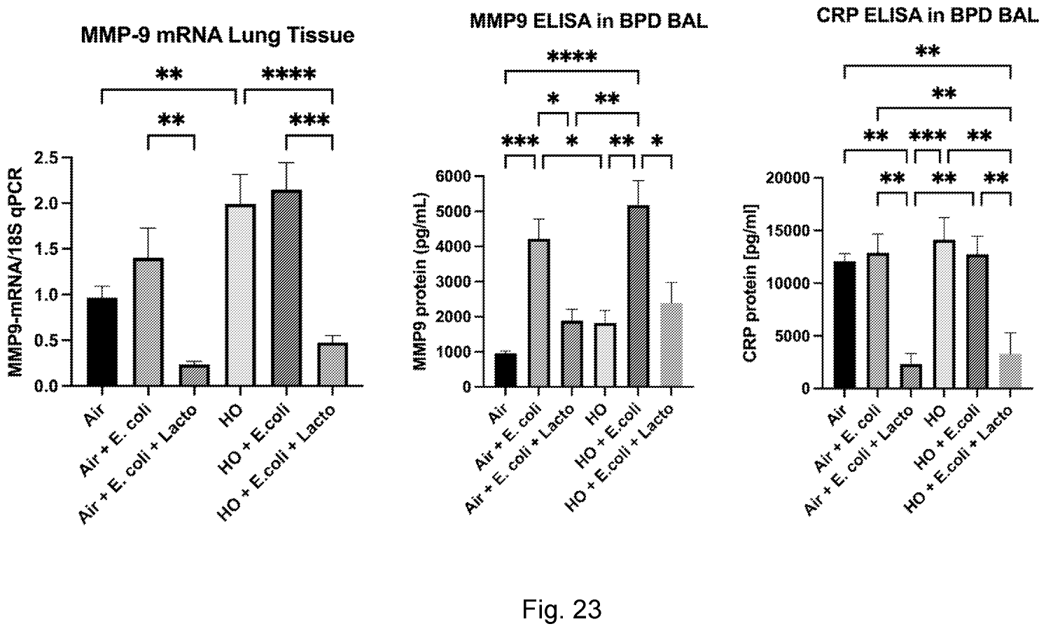

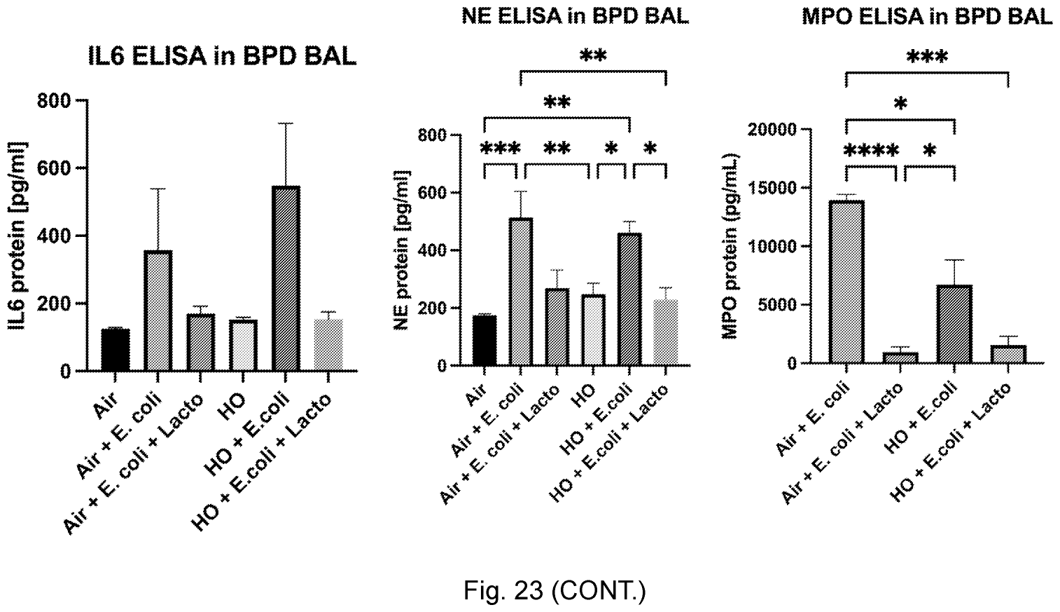

In FIG. 23, markers of neutrophilic inflammation were measured in a double hit hyperoxia (HO)+E. coli murine model of bronchopulmonary dysplasia (BPD).

In FIG. 24, a blend of live bacterial strains AB101, AB102, and AB103 was tested for its resistance to 49 antibiotics. In antibiotics in which the blend was resistant, individual strains were assayed for resistance (see also FIG. 8).

What is claimed herein is:

1. A spray-dried biotherapeutic matrix composition comprising a bacterial preparation, wherein the matrix composition is formulated for administration by inhalation.

2. The composition of claim 1, wherein the bacterial preparation comprises viable or non-viable bacteria.

3. The composition of claim 2, wherein the viable bacteria are capable of actively metabolizing and/or proliferating in the lung of a subject.

4. The composition of claim 2, wherein the non-viable bacteria are heat-killed.

5. The composition of claim 2, wherein the bacteria are Gram negative.

6. The composition of claim 2, wherein the bacteria are Gram positive.

7. The composition of claim 2, wherein the bacteria are spore-forming.

8. The composition of claim 2, wherein the bacteria are in spore form.

9. The composition of claim 2, wherein the bacteria are aerobic.

10. The composition of claim 2, wherein the bacteria are anaerobic.

11. The composition of claim 2, wherein the bacteria produce at least one immunomodulator.

12. The composition of claim 2, wherein the bacteria belong to a genus selected from the group consisting of: Carnobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella.

13. The composition of claim 2, wherein the bacterium is Lacticaseibacillus rhamnosus, Lactobacillus acidophilus, or Lactiplantibacillus plantarum.

14. The composition of claim 2, wherein the bacteria are non-pathogenic.

15. The composition of claim 2, wherein the bacteria are present at a concentration of at least 10¹ colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g.

16. The composition of claim 2, wherein the bacteria are present at a concentration of at least 10⁶ colony-forming units per gram (CFU/g).

17. The composition of claim 2, wherein the bacteria are present at a concentration of at least 10⁸ colony-forming units per gram (CFU/g).

18. The composition of claim 2, wherein the bacteria are resistant to at least one antibiotic.

19. The composition of claim 1, wherein the composition comprises at least 0.5% bacterial preparation by dry weight.

20. The composition of claim 1, wherein the bacterial preparation comprises a bacterial extract or bacterial metabolite preparation.

21. The composition of claim 20, wherein the bacterial extract or bacterial metabolite preparation is selected from the group consisting of: a bacterial exosome; bacterial cell wall; peptidoglycan; teichoic acid; lipoteichoic acid; bacterial S-layer; exopolysaccharide; polysaccharide; a lactic acid polymer; a lactic acid derivative; a lactic acid intermediate; hydrogen peroxide; a bacteriocin; a salivaricin; a reuterin; and a bacterial growth supernatant.

22. The composition of claim 1, further comprising at least one excipient.

23. The composition of claim 1, further comprising at least two excipients.

24. The composition of claim 22, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

25. The composition of claim 22, wherein the excipient is leucine and/or trehalose.

26. The composition of claim 1, wherein the composition comprises at least 0.5% excipient by dry weight.

27. The composition of claim 1, further comprising at least one stabilizer.

28. The composition of claim 27, wherein the stabilizer comprises a surfactant.

29. The composition of claim 27, wherein the stabilizer is a polysorbate; poloxamer; or polyvinyl alcohol.

30. The composition of claim 27, wherein the stabilizer is Polysorbate 20, Polysorbate 40, Polysorbate 60, or Polysorbate 80.

31. The composition of claim 27, wherein the stabilizer is Polysorbate 80.

32. The composition of claim 27, wherein the stabilizer is Poloxamer 184, Poloxamer 185, Poloxamer 188, Poloxamer 234, Poloxamer 235, Poloxamer 238, Poloxamer 333, Poloxamer 334, Poloxamer 335, Poloxamer 338, Poloxamer 403, or Poloxamer 407.

33. The composition of claim 1, wherein the composition comprises at least 0.25% of the stabilizer or stabilizers by dry weight.

34. The composition of claim 1, wherein the composition comprises at least one excipient and at least one stabilizer.

35. The composition of claim 1, wherein the composition further comprises at least one additional therapeutic.

36. The composition of claim 35, wherein the at least one additional therapeutic is selected from the group consisting of: an anti-inflammatory, an antimicrobial, an antiviral, an antifungal, a vasodilator, and a bronchodilator.

37. The composition of claim 36, wherein the at least one additional therapeutic is incorporated into the composition using microencapsulation, co-formulation, or covalent linkage to the composition with a degradable linker.

38. The composition of claim 1, wherein the matrix composition comprises a plurality of dried particles.

39. The composition of claim 38, wherein the dried particles have a Dv50 of at least 0.5 μm.

40. The composition of claim 38, wherein the dried particles have a median mass aerodynamic diameter (MMAD) of at least 1.5 μm to at most 7.5 μm.

41. The composition of claim 38, wherein the dried particles have a dispersibility of less than 2.0.

42. The composition of claim 38, wherein the dried particles have a dispersibility of at least 0.5 to 1.0.

43. The composition of claim 38, wherein the dried particles have a delivered dose of at least 25.0% to at most 125% of the bacterial preparation by mass to a target tissue.

44. The composition of claim 38, wherein the dried particles have a delivered dose of at least 60% of the bacterial preparation by mass to a target tissue.

45. The composition of claim 44, wherein the target tissue is a target bronchopulmonary tissue.

46. The composition of claim 45, wherein the target bronchopulmonary tissue is the lungs, the trachea, the bronchi, the bronchioles, and/or the alveoli.

47. The composition of claim 45, wherein the target tissue is a distal tissue site from the lungs delivered via the cardiovascular system or lymphatic system.

48. The composition of claim 38, wherein the dried particles have a bulk density of at least 0.1 g/cm³ to 0.8 g/cm³.

49. The composition of claim 38, wherein the dried particles have a bulk density of at least 0.5 g/cm³.

50. The composition of claim 38, wherein the dried particles have a tapped density of at least 0.1 g/cm³ to 1.0 g/cm³.

51. The composition of claim 38, wherein the dried particles have a tapped density of at least 0.6 g/cm³.

52. The composition of claim 38, wherein the dried particles have a moisture content of at least 1.0% to 7.0% water by weight by Karl Fischer.

53. The composition of claim 38, wherein the dried particles have a moisture content of at least 2.5% water by weight by Karl Fischer.

54. The composition of claim 1, wherein the composition is formulated for delivery to the trachea, the bronchi, the bronchioles, and/or the alveoli.

55. The composition of claim 1, wherein the composition is formulated for delivery to the lungs.

56. The composition of claim 1, wherein the composition is formulated as a capsule.

57. The composition of claim 56, wherein the capsule contains at least 10 mg of the spray-dried biotherapeutic matrix composition.

58. The composition of claim 1, wherein the composition is formulated for delivery by an inhaler.

59. The composition of claim 1, wherein the composition is formulated for delivery by a dry powder inhaler (DPI), a metered dose inhaler (MDI), or a soft-mist inhaler (SMI).

60. The composition of claim 1, in combination with an inhaler.

61. An inhalation device for bronchopulmonary delivery comprising:
a) an inhaler; and
b) a container containing a spray-dried biotherapeutic matrix composition comprising a bacterial preparation.

62. The device of claim 61, wherein the inhaler is a dry powder inhaler (DPI), a metered dose inhaler (MDI), or a soft mist inhaler (SMI).

63. The device of claim 61, wherein the inhaler comprises:
a) a mouthpiece comprising an opening; and
b) means for aerosolizing or dispersing the spray-dried biotherapeutic matrix composition in the container.

64. A method of preparing a spray-dried biotherapeutic matrix composition comprising a bacterial preparation, comprising:
a) preparing a liquid feedstock comprising the bacterial preparation;
b) introducing droplets of the liquid feedstock through an atomization nozzle into a drying chamber;
c) exposing the liquid feedstock droplets to heated, pressurized gas in the drying chamber to create dried particles; and
d) isolating dried particles of a predetermined range of diameters in a cyclone chamber, wherein the isolated dried particles comprise the spray-dried biotherapeutic matrix composition.

65. A method of preparing a spray-dried biotherapeutic matrix composition comprising a bacterial preparation, comprising:
a) obtaining a liquid feedstock comprising the bacterial preparation;
b) introducing droplets of the liquid feedstock through an atomization nozzle into a drying chamber;
c) exposing the liquid feedstock droplets to heated, pressurized gas in the drying chamber to create dried particles; and
d) isolating dried particles of a predetermined range of diameters in a cyclone chamber, wherein the isolated dried particles comprise the spray-dried biotherapeutic matrix composition.

66. The method of claim 64, wherein the step of preparing the liquid feedstock comprises dissolving a solid feedstock into an aqueous solution.

67. The method of claim 64, wherein the solid feedstock comprises:
a) at least 3.5% bacterial preparation by weight;
b) at least 5% excipient by weight; and/or
c) at least 0.25% stabilizer by weight.

68. The method of claim 64, wherein the solid feedstock comprises:
a) at least 3.5% bacterial preparation by weight;
b) at least 5% of a first excipient by weight;
c) at least 5% of a second excipient by weight; and/or
d) at least 0.25% stabilizer by weight.

69. The method of claim 64, wherein the solid feedstock comprises at least 1% to at most 5% bacterial preparation by weight.

70. The method of claim 64, wherein the solid feedstock comprises at least 45% to at most 95% excipient by weight.

71. The method of claim 64, wherein the solid feedstock comprises at least 5%-60% of a first excipient by weight, and at least 5%-60% of a second excipient by weight.

72. The method of claim 64, wherein the solid feedstock comprises at least 0.25% to at most 10% stabilizer by weight.

73. The method of claim 64, wherein the liquid feedstock comprises at least 1 g/L solid feedstock dissolved in an aqueous solution.

74. The method of claim 64, wherein the liquid feedstock comprises at least 25 g/L solid feedstock dissolved in an aqueous solution.

75. The method of claim 64, wherein the liquid feedstock comprises at least 0.1% to at most 10% solid feedstock dissolved in an aqueous solution.

76. The method of claim 64, wherein the liquid feedstock comprises at least 1% solid feedstock dissolved in an aqueous solution.

77. The method of claim 64, wherein 1 L of the liquid feedstock comprises:
a) at least 1.050 g bacterial preparation;
b) at least 1.5 g excipient;
c) at least 0.075 g stabilizer; and/or
d) at least 970 g aqueous solution.

78. The method of claim 64, wherein 1 L of the liquid feedstock comprises:
a) at least 1.050 g bacterial preparation;
b) at least 0.75 g of a first excipient;
c) at least 0.75 g of a second excipient;
d) at least 0.075 g stabilizer; and/or
e) at least 970 g aqueous solution.

79. The method of claim 64, wherein 1 L of the liquid feedstock comprises:
a) at least 1.050 g bacterial preparation;
b) at least 0.5 g of a first excipient;
c) at least 0.5 g of a second excipient;
d) at least 0.5 g of a third excipient;
e) at least 0.075 g stabilizer; and/or
f) at least 970 g aqueous solution.

80. The method of claim 64, wherein 1 L of the liquid feedstock comprises at least 750 g to at most 999 g aqueous solution.

81. The method of claim 64, wherein the liquid feedstock comprises:
a) at least 0.105% bacterial preparation;
b) at least 0.15% excipient;
c) at least 0.0075% stabilizer; and/or
d) at least 97% aqueous solution.

82. The method of claim 64, wherein the liquid feedstock comprises:
a) at least 0.105% bacterial preparation by weight;
b) at least 0.075% of a first excipient by weight;
c) at least 0.075% of a second excipient by weight;
d) at least 0.0075% stabilizer and/or
e) at least 97% aqueous solution by weight.

83. The method of claim 64, wherein the liquid feedstock comprises:
a) at least 0.105% bacterial preparation by weight;
b) at least 0.05% of a first excipient by weight;
c) at least 0.05% of a second excipient by weight;
d) at least 0.05% of a third excipient by weight;
e) at least 0.0075% stabilizer and/or
f) at least 97% aqueous solution by weight.

84. The method of claim 64, wherein the liquid feedstock comprises at least 0.01% to at most 10% bacterial preparation by weight.

85. The method of claim 64, wherein the liquid feedstock comprises at least 1.0% to at most 20% excipient by weight.

86. The method of claim 64, wherein the liquid feedstock comprises at least 0.10% to at most 19.8% of a first excipient by weight, and at least 0.1% to at most 19.8% of a second excipient by weight.

87. The method of claim 64, wherein the liquid feedstock comprises at least 0.10% to at most 19.8% of a first excipient by weight, at least 0.1% to at most 19.8% of a second excipient by weight, and at least 0.1% to at most 19.8% of a third excipient by weight.

88. The method of claim 64, wherein the liquid feedstock comprises at least 0.01% to at most 1.0% stabilizer by weight.

89. The method of claim 64, wherein the liquid feedstock comprises at least 75% to at most 99.9% aqueous solution by weight.

90. The method of claim 64, wherein the bacterial preparation comprises viable or non-viable bacteria.

91. The method of claim 90, wherein the bacteria belong to a genus selected from the group consisting of: Carnobacterium; Lactiplantibacillus; Lactobacillus; Lacticaseibacillus; Ligilactobacillus; Oenococcus; Leuconostoc; Pedicoccus; Enterococcus; Lactococcus; Staphylococcus; Streptococcus; Streptomyces; Bifidobacterium; Propionibacterium; and Moraxella.

92. The method of claim 90, wherein the bacterium is Lacticaseibacillus rhamnosus, Lactobacillus acidophilus, or Lactiplantibacillus plantarum.

93. The method of claim 90, wherein the bacteria are non-pathogenic.

94. The method of claim 90, wherein the bacteria are present at a concentration of at least 10¹ colony-forming units per gram (CFU/g), at least 10² CFU/g, at least 10³ CFU/g, at least 10⁴ CFU/g, at least 10⁵ CFU/g, at least 10⁶ CFU/g, at least 10⁷ CFU/g, at least 10⁸ CFU/g, at least 10⁹ CFU/g, at least 10¹⁰ CFU/g, at least 10¹¹ CFU/g, or at least 10¹² CFU/g.

95. The method of claim 90, wherein the bacteria are present at a concentration of at least 10⁶ colony-forming units per gram (CFU/g).

96. The method of claim 90, wherein the bacteria are present at a concentration of at least 10⁸ colony-forming units per gram (CFU/g).

97. The method of claim 90, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcamitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

98. The method of claim 64, wherein the excipient is leucine and/or trehalose.

99. The method of claim 64, wherein the stabilizer is a polysorbate; poloxamer; or polyvinyl alcohol.

100. The method of claim 64, wherein the stabilizer is Polysorbate 80.

101. The method of claim 64, wherein the aqueous solution is water.

102. The method of claim 64, wherein the liquid feedstock further comprises at least one additional therapeutic.

103. The method of claim 102, wherein the at least one additional therapeutic is selected from the group consisting of: an anti-inflammatory, an antimicrobial, an antiviral, an antifungal, a vasodilator, and a bronchodilator.

104. The method of claim 64, wherein the atomization nozzle into the drying chamber has a diameter of at least 1.2 um.

105. The method of claim 64, wherein the droplets of liquid feedstock produced by the atomization nozzle into the drying chamber have a diameter of at least 1.5 um.

106. The method of claim 64, wherein the droplets of liquid feedstock have a flow rate through the drying chamber of at least 0.5 g/min.

107. The method of claim 64, wherein the droplets of liquid feedstock have a flow rate through the drying chamber of at least 15 g/min.

108. The method of claim 64, the droplets of liquid feedstock have a flow rate through the drying chamber of at most 1000 g/min.

109. The method of claim 64, wherein the heated, pressurized gas is heated before being inlet into the drying chamber.

110. The method of claim 64, wherein the heated, pressurized gas is inlet into the drying chamber at a temperature of at least 100° C.

111. The method of claim 64, wherein the heated, pressurized gas is inlet into the drying chamber at a temperature of at most 195° C.

112. The method of claim 64, wherein the heated, pressurized gas is outlet from the drying chamber at a temperature of at least 40° C.

113. The method of claim 64, wherein the heated, pressurized gas is outlet from the drying chamber at a temperature of at least 48° C.

114. The method of claim 64, wherein the heated, pressurized gas is outlet from the drying chamber at a temperature of at most 85° C.

115. The method of claim 64, wherein the heated, pressurized gas is pressurized before being inlet into the drying chamber.

116. The method of claim 64, wherein the heated, pressurized gas in the drying chamber has an atomization gas pressure of at least 10 pounds per square inch gauge (psig).

117. The method of claim 64, wherein the heated, pressurized gas in the drying chamber has an atomization gas pressure of at least 20 pounds per square inch gauge (psig).

118. The method of claim 64, wherein the heated, pressurized gas in the drying chamber has an atomization gas pressure of at most 150 pounds per square inch gauge (psig).

119. The method of claim 64, wherein the heated, pressurized gas has a flow rate through the drying chamber of at least 5 kg/hr.

120. The method of claim 64, wherein the heated, pressurized gas has a flow rate through the drying chamber of at least 18 kg/hr.

121. The method of claim 64, wherein the heated, pressurized gas has a flow rate through the drying chamber of at most 150 kg/hr.

122. The method of claim 64, wherein the heated, pressurized gas is outlet through the cyclone chamber.

123. The method of claim 64, wherein the step of exposing the liquid feedstock droplets to heated, pressurized gas in the drying chamber takes at most 8 hours.

124. The method of claim 64, wherein the dried particles isolated in the cyclone chamber have a median mass aerodynamic diameter (MMAD) of at least 1.5 μm to at most 7.5 μm.

125. The method of claim 64, wherein the dried particles isolated in the cyclone chamber have a median mass aerodynamic diameter (MMAD) of at least 4.0 μm.

126. The method of claim 64, wherein the step of isolating dried particles of a predetermined range of diameters in the cyclone chamber occurs continuously.

127. The method of claim 64, wherein the spray-dried biotherapeutic matrix composition comprises a bacterial viability of at least 5% after the step of isolating the dried particles.

128. A method of delivering a spray-dried biotherapeutic matrix composition comprising a bacterial preparation to a subject, comprising:
a) obtaining an inhalation device for bronchopulmonary delivery comprising:
i) an inhaler; and
ii) a container containing a spray-dried biotherapeutic matrix composition comprising a bacterial preparation;
b) activating the inhaler to cause aerosolization or dispersal of the spray-dried biotherapeutic matrix composition; and
d) inhaling the aerosolized or dispersed spray-dried biotherapeutic matrix composition.

129. A method of delivering a spray-dried biotherapeutic matrix composition comprising a bacterial preparation to a subject, comprising:
a) obtaining an inhalation device for bronchopulmonary delivery comprising:
i) an inhaler; and
ii) a container containing the spray-dried biotherapeutic matrix composition of claim 1;
d) activating the inhaler to cause aerosolization or dispersal of the spray-dried biotherapeutic matrix composition; and
e) inhaling the aerosolized or dispersed spray-dried biotherapeutic matrix composition.

130. A method of delivering a spray-dried biotherapeutic matrix composition comprising a bacterial preparation to a subject, comprising:
a) obtaining the inhalation device of claim 61;
c) activating the inhaler to cause aerosolization or dispersal of the spray-dried biotherapeutic matrix composition; and
d) inhaling the aerosolized or dispersed spray-dried biotherapeutic matrix composition.

131. The method of claim 128, wherein the inhaler is a dry powder inhaler (DPI), a metered dose inhaler (MDI), or a soft mist inhaler (SMI).

132. The method of claim 128, wherein the inhaler comprises:
a) a mouthpiece comprising an opening; and
b) means for aerosolizing or dispersing the spray-dried biotherapeutic matrix composition in the container.

133. The method of claim 128, wherein the inhaler has an inspiration flow rate of at least 15 L/min.

134. The method of claim 128, wherein at least 25% to at most 100% of the spray-dried biotherapeutic matrix composition by mass is delivered to a target bronchopulmonary tissue.

135. The method of claim 128, wherein at least 60.0% of the spray-dried biotherapeutic matrix composition by mass is delivered to a target bronchopulmonary tissue.

136. The method of claim 128, wherein the target bronchopulmonary tissue is the lungs, the trachea, the bronchi, the bronchioles, and/or the alveoli.

137. The method of claim 128, wherein the spray-dried biotherapeutic matrix composition is delivered from the bronchopulmonary tissue to a distal tissue site via the cardiovascular system or lymphatic system.

138. A method of treating a subject in need thereof comprising administering through inhalation an effective dose of a spray-dried biotherapeutic matrix composition comprising a bacterial preparation.

139. A method of treating a subject in need thereof comprising administering through inhalation an effective dose of the spray-dried biotherapeutic matrix composition of claim 1.

140. The method of claim 138, wherein the subject has been diagnosed with or is at risk of developing a chronic bronchopulmonary disease.

141. The method of claim 138, wherein the chronic bronchopulmonary disease is selected from the group consisting of: chronic obstructive pulmonary disease (COPD), lung cancer, asthma, bronchiectasis, emphysema, cystic fibrosis (CF), bronchopulmonary dysplasia (BPD), acute respiratory disease syndrome (ARDS), idiopathic pulmonary fibrosis (IPF), pulmonary hypertension (PAH), silicosis, interstitial lung disease (ILD), and pleural effusion (PE).

142. The method of claim 138, wherein the lung cancer is small cell lung cancer (SCLC) or non-small cell lung cancer (NSCLC).

143. The method of claim 138, wherein the subject has been diagnosed with or is at risk of contracting an infectious pulmonary disease.

144. The method of claim 138, wherein the infectious bronchopulmonary disease is caused by or associated with an infectious agent selected from: adenovirus; coronavirus; influenza virus; parainfluenza virus; parvovirus; respiratory syncytial virus; rhinovirus; enterovirus; measles virus; rubella virus; varicella virus; Corynebacterium diphtheriae; Haemophilus influenzae; Legionella pneumophila; Bordetella pertussis; Mycobacterium tuberculosis; Streptococcus species; Pseudomonas species; Escherichia coli; Aspergillus species; Cryptococcus species; and Pneumocystis species.

145. The method of claim 138, wherein the spray-dried biotherapeutic matrix composition is administered in conjunction with a standard of care for the chronic or infectious bronchopulmonary disease.

146. The method of claim 138, wherein the spray-dried biotherapeutic matrix composition is administered using an inhaler.

147. The method of claim 138, wherein the inhaler is a dry powder inhaler (DPI), a metered dose inhaler (MDI), or a soft mist inhaler (SMI).

148. The method of claim 138, wherein the effective dose of the spray-dried biotherapeutic matrix composition is at least 10⁴ CFU of bacteria per unit dose.

149. The method of claim 138, wherein the effective dose of the spray-dried biotherapeutic matrix composition is at least 10⁴ CFU of viable bacteria per unit dose.

150. The method of claim 138, wherein the spray-dried biotherapeutic matrix composition reduces neutrophilic inflammation in a target tissue.

151. The method of claim 138, wherein the spray-dried biotherapeutic matrix composition increases lactic acid concentration in a target tissue by at least 25%.

152. The method of claim 138, further comprising administering at least one additional therapeutic.

153. The method of claim 152, wherein the at least one additional therapeutic is selected from the group consisting of an anti-inflammatory, an antimicrobial, an antiviral, an antifungal, a vasodilator, and a bronchodilator.

154. The method of claim 152, wherein the spray-dried biotherapeutic matrix composition comprises the bacterial preparation and the at least one additional therapeutic.

155. The method of claim 152, wherein the spray-dried biotherapeutic matrix composition is co-administered with the at least one additional therapeutic.

156. The method of claim 152, wherein the co-administration comprises administering using a combination delivery device.

157. A unit dosage form comprising at least 1 mg of a spray-dried biotherapeutic matrix composition comprising a bacterial preparation.

158. A unit dosage form comprising at least 1 mg of the spray-dried biotherapeutic matrix composition of claim 1.

159. A unit dosage form comprising at least 1 mg of the spray-dried biotherapeutic matrix composition prepared by the methods of claim 64-.

160. A unit dosage form comprising at least 1 mg of a spray-dried biotherapeutic matrix composition comprising at least 10⁴ CFU bacteria per unit dose.

161. The unit dosage form of claim 157, wherein the dosage is at least 30 mg spray-dried biotherapeutic matrix composition.

162. The unit dosage form of claim 157, wherein the dosage comprises at least 10⁴ CFU of bacteria per unit dose.

163. The unit dosage form of claim 157, wherein the dosage comprises at least 10⁴ CFU of viable bacteria per unit dose.
